(19) United States
(12) Patent Application Publication
(10) Pub. No.: US 2013/0189759 A1
(43) Pub. Date: Jul. 25, 2013

(54) MEGANUCLEASES VARIANTS CLEAVING A DNA TARGET SEQUENCE IN THE NANOG GENE AND USES THEREOF

(71) Applicant: David Sourdive, Levallois-Perret (FR)

(72) Inventor: David Sourdive, Levallois-Perret (FR)

(73) Assignee: Cellectis, Paris (FR)

(21) Appl. No.: 13/808,745

(22) PCT Filed: Jul. 7, 2011
(86) PCT No.: PCT/IB11/01934

§ 371 Date: Mar. 26, 2013

Related U.S. Application Data
(60) Provisional application No. 61/362,234, filed on Jul. 7, 2010.

(51) Int. Cl. C12N 9/16

(52) U.S. Cl. CPC C12N 9/16
USPC 435/196; 435/325; 435/377; 435/320.1; 536/23.2

(57) ABSTRACT

Meganuclease variants cleaving DNA target sequences of the NANOG gene, vectors encoding such variants, and cells expressing them. Methods of using meganuclease variants recognizing NANOG gene sequences for modifying the NANOG gene sequence or for incorporating a gene of interest or therapeutic gene using the NANOG gene as a landing pad and a safe harbor locus.

### CROSS-REFERENCE TO RELATED APPLICATIONS

(not applicable)

### STATEMENT REGARDING FEDERALLY SPONSORED RESEARCH OR DEVELOPMENT

(not applicable)

### REFERENCE TO MATERIAL ON COMPACT DISK

(not applicable)

### BACKGROUND OF THE INVENTION

1. Field of the Invention

The present invention concerns a process to generate new class of induced Pluripotent Stem (iPS) cells and their derivatives characterized as clean and/or safe and/or secure by using endonucleases such as meganucleases and particularly the meganucleases of the present invention.

2. Description of the Related Art

NANOG, a name reportedly derived from the Tir na nOg legend describing a Land of Youth, is a gene involved in the self-renewal of embryonic stem cell (ES cell) which are pluripotent cells. Pluripotent cells have the capacity to differentiate into cells forming all three of the basic germ cell layers, endoderm, mesoderm and ectoderm and to cells subsequently differentiating from these layers.

The NANOG gene is located on chromosome XII of the human genome and composed of four exons which range in length between 87 and 417 bp. With 3 introns, the total gene sequence is 6,661 bp. NANOG is a key gene implied in self-renewal properties of pluripotent stem cells, embryonic stem cells (ES) or induced pluripotent stem cells (iPS). Pluripotent stem cells are cells capable to self-renew indefinitely and are pluripotent: they can be differentiated into all cell types of the body. These two properties make pluripotent stem cells good candidates for cell therapy, drug screening studies and for the production of iPS or ES seed lots.

NANOG gene, polynucleotide and amino acid sequences are well-known in the art and are also incorporated by reference for human NANOG sequences and for other mammalian NANOG sequences. As used herein, the term NANOG gene includes regulatory sequences outside of the NANOG coding sequence, such as promoter or enhancer sequences or regulatory sequences. NANOG contains a homeodomain spanning residues that binds to DNA and RNA.

Embryonic stem cells can be derived from an embryo, such as a discarded embryo resulting from an in vitro fertilization procedure. In distinction, induced Pluripotent Stem cells or iPS cells are generated from somatic cells by the introduction of four transcription factors (e.g. Oct4, Sox2, c-Myc, Klf4) (Takahashi, et al., 2006, 2007).

The NANOG gene has been demonstrated to play a role in cellular reprogramming processes (Yu, et al., 2007). Its expression is a criterion for the validation of truly reprogrammed cells (Silva, et al., 2008, 2009). The role of NANOG in pluripotent stem cells has been identified by over-expression and knock-down experiments. Notably, it has been shown that over-expression of NANOG in mouse ES cells causes them to self-renew in the absence of Leukemia inhibitory factor an otherwise essential factor for mouse ES cells culture. In the absence of NANOG, mouse ES cells differentiate into visceral/parietal endoderm and loss of NANOG function causes differentiation of mouse ES cells into other cell types (Chambers, et al, 2003).

Similarly, in human ES cells, NANOG over-expression enables their propagation for multiple passages during which the cells remain pluripotent. Gene knockdown of NANOG promotes differentiation, thereby demonstrating a role for this factor in human ES cell self-renewal. In addition, NANOG is thought to function in concert with other factors such as OCT4 and SOX2 to establish ES cell identity (Dan, et al., 2006, Li, et al., 2007).

Homologous gene targeting strategies have been used to knock out endogenous genes (WO90/11354 (Capecchi 1989; Smithies 2001) or knock-in exogenous sequences into the genome. To enhance the efficiency of gene targeting, another strategy to enhance its efficiency is to deliver a DNA double-strand break (DSB) in the targeted locus, using an enzymatically induced double strand break at or around the locus where recombination is required (WO96/14408). A strategy known as “exon knock-in” involves the use of a meganuclease cleaving a targeted gene sequence to knock-in a functional exonic sequences. Meganucleases have been identified as suitable enzymes to induce the required double-strand break. Meganucleases are by definition sequence-specific endonucleases recognizing large sequences (Thierry, A. and B. Dujon, Nucleic Acids Res., 1992, 20, 5625-5631). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. U.S.A., 1996, 93, 5055-5060; Sargent et al., Mol. Cell. Biol., 1997, 17, 267-277; Cohen-Tannoudji et al., Mol. Cell. Biol., 1998, 18, 1444-1448; Donoho, et al., Mol. Cell. Biol., 1998, 18, 4070-4078; Elliott et al., Mol. Cell. Biol., 1998, 18, 93-101).

Although several hundred natural meganucleases, also referred to as “homing endonucleases” have been identified (Chevalier, B. S. and B. L. Stoddard, Nucleic Acids Res., 2001, 29, 3757-3774), the repertoire of cleavable target sequences is too limited to allow the specific cleavage of a target site in a gene of interest or GOI as there is usually no cleavable site in a chosen gene of interest. For example, there is no cleavage site for known naturally occurring I-Cre1 or I-Sce1 meganucleases in human NANOG.

Theoretically, the making of artificial sequence-specific endonucleases with chosen specificities could alleviate this limit. To overcome this limitation, an approach adopted by a number of workers in this field is the fusion of Zinc-Finger Proteins (ZFPs) with the catalytic domain of FokI, a class IIS restriction endonuclease, so as to make functional sequence-specific endonucleases (Smith et al., Nucleic Acids Res., 1999, 27, 674-681; Bibikova et al., Mol. Cell. Biol., 2001, 21, 289-297; Bibikova et al., Genetics, 2002, 161, 1169-1175; Bibikova et al., Science, 2003, 300, 764; Porteus, M. H. and D. Baltimore, Science, 2003, 300, 763-; Alwin et al., Mol. Ther., 2005, 12, 610-617; Urnov et al., Nature, 2005, 435, 646-651; Porteus, M. H., Mol. Ther., 2006, 13, 438-446). Such ZFP nucleases have been used for the engineering of the IL2RG gene in human lymphoid cells (Urnov et al., Nature, 2005, 435, 646-651).

The binding specificity of Cys2-His2 type Zinc-Finger Proteins, is easy to manipulate because specificity is driven by essentially four residues per zinc finger (Pabo et al., Annu. Rev. Biochem., 2001, 70, 313-340; Jamieson et al., Nat. Rev. Drug Discov., 2003, 2, 361-368). Studies from the Pabo laboratories have resulted in a large repertoire of novel artificial ZFPs, able to bind most G/ANNG/ANNG/ANN sequences (Rebar, E. J. and C. O. Pabo, Science, 1994, 263, 671-673; Kim, J. S. and C. O. Pabo, Proc. Natl. Acad. Sci. USA, 1998, 95, 2812-2817), Klug (Choo, Y. and A. Klug, Proc. Natl. Acad. Sci. USA, 1994, 91, 11163-11167; Isalan M. and A. Klug, Nat. Biotechnol., 2001, 19, 656-660) and Barbas (Choo, Y. and A. Klug, Proc. Natl. Acad. Sci. USA, 1994, 91, 11163-11167; Isalan M. and A. Klug, Nat. Biotechnol., 2001, 19, 656-660).

Nevertheless, ZFPs have serious limitations, especially for applications requiring a very high level of specificity, such as therapeutic applications. It was shown that FokI nuclease activity in ZFP fusion proteins can act with either one recognition site or with two sites separated by variable distances via a DNA loop (Catto et al., Nucleic Acids Res., 2006, 34, 1711-1720). Thus, the specificities of these ZFP nucleases are degenerate, as illustrated by high levels of toxicity in mammalian cells and Drosophila (Bibikova et al., Genetics, 2002, 161, 1169-1175; Bibikova et al., Science, 2003, 300, 764-; Hockemeyer et al., Nat. Biotechnol. 2009 September; 27(9): 851-7).

The inventors have discovered and adopted a new approach which circumvents these problems using engineered endonucleases, such as meganucleases recognizing NANOG gene sequences.

In the wild, meganucleases are essentially represented by homing endonucleases. Homing Endonucleases (HEs), a widespread family of natural meganucleases including hundreds of proteins families (Chevalier, B. S. and B. L. Stoddard, Nucleic Acids Res., 2001, 29, 3757-3774). These proteins are encoded by mobile genetic elements which propagate by a process called “homing”: the endonuclease cleaves a cognate allele from which the mobile element is absent, thereby stimulating a homologous recombination event that duplicates the mobile DNA into the recipient locus. Given their exceptional cleavage properties in terms of efficacy and specificity, they could represent ideal scaffold to derive novel, highly specific endonucleases.

Homing Endonucleases belong to four major families. The LAGLIDADG family, named after a conserved peptidic motif involved in the catalytic center, is the most widespread and the best characterized group. Seven structures are now available. Whereas most proteins from this family are monomeric and display two LAGLIDADG motifs, a few have only one motif, but dimerize to cleave palindromic or pseudo-palindromic target sequences.

Although the LAGLIDADG peptide is the only conserved region among members of the family, these proteins share a very similar architecture. The catalytic core is flanked by two DNA-binding domains with a perfect two-fold symmetry for homodimers such as I-CreI (Chevalier, et al., Nat. Struct. Biol., 2001, 8, 312-316) and I-MsoI (Chevalier et al., J. Mol. Biol., 2003, 329, 253-269) and with a pseudo symmetry for monomers such as I-SceI (Moure et al., J. Mol. Biol., 2003, 334, 685-69, I-DmoI (Silva et al., J. Mol. Biol., 1999, 286, 1123-1136) or I-AniI (Bolduc et al., Genes Dev., 2003, 17, 2875-2888).

Both monomers or both domains of monomeric proteins contribute to the catalytic core, organized around divalent cations. Just above the catalytic core, the two LAGLIDADG peptides play also an essential role in the dimerization interface. DNA binding depends on two typical saddle-shaped αββαββα folds, sitting on the DNA major groove. Other domains can be found, for example in inteins such as PI-PfuI (Ichiyanagi et al., J. Mol. Biol., 2000, 300, 889-901) and PI-SceI (Moure et al., Nat. Struct. Biol., 2002, 9, 764-770), which protein splicing domain is also involved in DNA binding.

The making of functional chimeric meganucleases by fusing the N-terminal I-DmoI domain with an I-CreI monomer have demonstrasted the plasticity of meganucleases (Chevalier et al., Mol. Cell., 2002, 10, 895-905; Epinat et al., Nucleic Acids Res, 2003, 31, 2952-62; International PCT Applications WO 03/078619 and WO 2004/031346).

Different groups have used a semi-rational approach to locally alter the specificity of I-CreI (Seligman et al., Genetics, 1997, 147, 1653-1664; Sussman et al., J. Mol. Biol., 2004, 342, 31-41; International PCT Applications WO 2006/097784 and WO 2006/097853; Arnould et al., J. Mol. Biol., 2006, 355, 443-458; Rosen et al., Nucleic Acids Res., 2006, 34, 4791-4800; Smith et al., Nucleic Acids Res., 2006, 34, e149), I-SceI (Doyon et al., J. Am. Chem. Soc., 2006, 128, 2477-2484), PI-SceI (Gimble et al., J. Mol. Biol., 2003, 334, 993-1008) and I-MsoI (Ashworth et al., Nature, 2006, 441, 656-659).

In addition, hundreds of I-CreI derivatives with locally altered specificity were engineered by combining the semi-rational approach and High Throughput Screening:
Residues Q44, R68 and R70 or Q44, R68, D75 and 177 of I-CreI were mutagenized and a collection of variants with altered specificity at positions±3 to 5 of the DNA target (5NNN DNA target) were identified by screening (International PCT Applications WO 2006/097784 and WO 2006/097853; Arnould et al., J. Mol. Biol., 2006, 355, 443-458; Smith et al., Nucleic Acids Res., 2006, 34, e149).
Residues K28, N30 and Q38 or N30, Y33, and Q38 or K28, Y33, Q38 and S40 of 1-CreI were mutagenized and a collection of variants with altered specificity at positions±8 to 10 of the DNA target (10NNN DNA target) were identified by screening (Smith et al., Nucleic Acids Res., 2006, 34, e149; International PCT Applications WO 2007/060495 and WO 2007/049156).

Two different variants were combined and assembled in a functional heterodimeric endonuclease able to cleave a chimeric target resulting from the fusion of a different half of each variant DNA target sequence (Arnould et al., precited; International PCT Applications WO 2006/097854 and WO 2007/034262). Interestingly, the novel proteins had kept proper folding and stability, high activity, and a narrow specificity.

Furthermore, residues 28 to 40 and 44 to 77 of I-CreI were shown to form two separable functional subdomains, able to bind distinct parts of a homing endonuclease half-site (Smith et al. Nucleic Acids Res., 2006, 34, e149; International PCT Applications WO 2007/049095 and WO 2007/057781).

The combination of mutations from the two subdomains of I-CreI within the same monomer allowed the design of novel chimeric molecules able to cleave a palindromic combined DNA target sequence comprising the nucleotides at positions ±3 to 5 and ±8 to 10 which are bound by each subdomain (Smith et al., Nucleic Acids Res., 2006, 34, e149; International PCT Applications WO 2007/060495 and WO 2007/049156), as illustrated on FIG. 2b.

The combination of the two former steps allows a larger combinatorial approach, involving four different subdomains. The different subdomains can be modified separately and combined to obtain an entirely redesigned meganuclease variant (heterodimer or single-chain molecule) with chosen specificity. In a first step, couples of novel meganucleases are combined in new molecules (“half-meganucleases”) cleaving palindromic targets derived from the target one wants to cleave. Then, the combination of such “half-meganuclease” can result in a heterodimeric species cleaving the target of interest. The assembly of four sets of mutations into heterodimeric endonucleases cleaving a model target sequence or a sequence from different genes has been described in the following patent applications: XPC gene (WO2007093918), RAG gene (WO2008010093), HPRT gene (WO2008059382), beta-2 microglobulin gene (WO2008102274), Rosa26 gene (WO2008152523), Human hemoglobin beta gene (WO2009013622) and Human Interleukin-2 receptor gamma chain (WO2009019614).

These variants can be used to cleave genuine chromosomal sequences and have paved the way for novel perspectives in several fields including gene therapy.

However, even though the base-pairs ±1 and ±2 do not display any contact with the protein, it has been shown that these positions are not devoid of content information (Chevalier et al., J. Mol. Biol., 2003, 329, 253-269), especially for the base-pair ±1 and could be a source of additional substrate specificity (Argast et al., J. Mol. Biol., 1998, 280, 345-353; Jurica et al., Mol. Cell., 1998, 2, 469-476; Chevalier, B. S. and B. L. Stoddard, Nucleic Acids Res., 2001, 29, 3757-3774). In vitro selection of cleavable I-CreI target (Argast et al., precited) randomly mutagenized, revealed the importance of these four base-pairs on protein binding and cleavage activity. It has been suggested that the network of ordered water molecules found in the active site was important for positioning the DNA target (Chevalier et al., Biochemistry, 2004, 43, 14015-14026). In addition, the extensive conformational changes that appear in this region upon I-CreI binding suggest that the four central nucleotides could contribute to the substrate specificity, possibly by sequence dependent conformational preferences (Chevalier et al., 2003, precited).

The inventors have identified and developed novel endonucleases, such as meganucleases, targeting NANOG gene sequences, such as NANOG target sites NANOG2, a site within exon 2 of the NANOG gene, and NANOG4, a site within intron 1 of the NANOG gene, as non limiting examples. The novel endonucleases and particularly the meganucleases of the invention introduce double stranded breaks within the NANOG gene offering new opportunities to modify, modulate, and control NANOG gene expression, to detect NANOG gene expression, or to introduce transgenes into the NANOG gene locus.

### BRIEF SUMMARY OF THE INVENTION

The present invention concerns a process to generate new class of induced Pluripotent Stem (iPS) cells and their derivatives characterized as clean and/or safe and/or secure by using endonucleases such as meganucleases and particularly the meganucleases of the present invention.

Key issues of current protocols to generate iPS by introducing the four transcription factors Oct3/4, Sox2, KLF4 and c-myc are that:
these introductions are not controlled and lead to heterogenous populations of iPS cells where transgenes are not inserted at the same locus and/or not with the same copy number,
iPS cells express these four transgenes permanently leading to problems for further differentiation steps.

Endonucleases of the present invention are a tool of choice overcoming these classical issues allowing:
stable, robust and single copy targeted insertion of the four transgenes at a defined locus allowing a controlled generation of homogenous iPS populations in high quantity.
the possibility to remove the four transgenes once iPS have been generated without any scar on the genome (“pop-out”), for obtaining clean iPS in further re-differentiation steps and therapeutic uses.

Another issue addressed by endonucleases of the present invention is the possibility to generate secured iPS and to standardize well-defined but still empirical current protocols. By using meganucleases inducing the targeting and the disruption of Nanog gene as a non limiting example, at a defined step of differentiation process, the progression of iPS toward differentiation states is made irreversible and safe since infinite self-renewable property of these cells is lost.

Also, by using endonucleases to insert at a safe locus of the genome, genes of interest and particular inducible genes defined as essential for progression of iPS toward differentiated cells (growth factors, transcription factors), it is possible to standardize the differentiation steps of an iPS.

This endonuclease approach of iPS generation and differentiation open new avenues for screening molecules and/or genes in vitro:
in order to securize and standardize the iPS differentiation process, gene candidates from an expression library responsible or implicated in a defined differentiation step can be inserted at a safe locus of an iPS genome locus, by using meganucleases.
to screen chemical libraries for compounds on primary cells carrying or not a genetical defect.
in order to evaluate drug response at a single patient scale in pharmacogenomic approaches.
to confirm or invalidate strategies or chemicals derived from predictive methods and algorithms in predictive toxicology measures.

Also, endoanucleases can be the ideal tool to create reporter cell lines integrating at a safe locus, reporter gene fused to a promoter specific of a defined reprogrammation step in order to validate the iPS reprogrammation process. The same approach can be envisioned during the re-differentiation process, allowing to precisely control this process and create progenitor cells bank, still able to divide a limited number of times and known to be able to move through the body and migrate towards the tissue where they are needed; they are particularly useful for adult organisms therapy as they act as a repair system for the body without presenting the known transplantation problem of compatibility.

Regarding therapeutic uses, endonucleases are the ideal tool to target and correct in clean and safe iPS cells pathological gene defects before their reinjection in patient organisms as suggested above (Pâques F. and Duchateau P., Current Gene Therapy, 2007, 7, 49-66).

Any gene involved in the reprogrammation of iPS cells is part of the present invention and is a useful target of endonucleases according to the invention. The present invention also concerns a new type of iPS; clean and/or safe and/or secure iPS cells as a new product will not anymore express the product of any gene of interest targeted for the process of cleaning and securization of such iPS cells, after the process of cleaning and securization occurs in said iPS cells.

In particular, the invention involves meganuclease variants that target and cleave NANOG gene sequences, vectors encoding these variants, cells transformed with vectors encoding these meganuclease variants and methods for making a meganuclease variant through by expressing a polynucleotide encoding it. Methods for designing meganuclease variants recognizing the NANOG gene, including meganuclease variants recognizing the NANOG2 and NANOG4 DNA sequences. These variant meganucleases are used to investigate the function of the NANOG gene, follow its expression in undifferentiated or pluripotent cells as well as in differentiated cells by introducing knock out mutations into the NANOG gene or by introducing reporter genes or other genes of interest at the NANOG locus, possibly for the production of proteins. The meganuclease variants of the invention may also be used to modulate NANOG expression in a cell by interaction of this gene sequence with a meganuclease, for example, to control its phenotype, to knock down or control expression of NANOG in a cell such as a tumor cell, or in various other therapeutic or diagnostic applications.

A particular aspect of the invention is a meganuclease that can induce double stranded breaks in any gene involved in the reprogrammation process and particularly in the NANOG gene.

Another aspect of the invention involves using such a meganuclease recognizing NANOG sequences to knock out or modulate NANOG expression. FIG. 1 illustrates such a strategy. Different strategies can be implemented for knocking out the NANOG (FIG. 1).

Another aspect of the invention is the use of a meganuclease recognizing NANOG to introduce a gene of interest into the NANOG gene or locus. The gene of interest may be a reporter gene that permits the expression of NANOG to be determined or followed over time, said reporter gene being associated or not to a nucleotidic sequence which is introduced into the genome in order to add new potentialities or properties to targeted cells. Methods for determining the effects of non-NANOG genes or drug compounds on NANOG expression or activity may be evaluated using assays employing a reporter gene. Such methods are particularly valuable when applied to tumor or cancer cells that have been modified to incorporate a NANOG gene associated with a reporter. Alternatively, the gene of interest may be a therapeutic transgene other than NANOG which uses the NANOG locus as a safe harbor. Such therapeutic genes may be those that when coexpressed with NANOG provide a particular cell phenotype of maintain or promote a particular phase or stage of cellular differentiation.

Thus, a third associated aspect of the invention relates to the use of the NANOG gene locus as a “landing pad” to insert or modulate the expression of genes of interest.

### BRIEF DESCRIPTION OF THE DRAWINGS

FIG. 1 A, B, C and D illustrates different strategies for knocking out NANOG. The coding sequence can be mutated by non homologous end joining (NHEJ) using a meganuclease targeting a sequence in the open reading frame (FIG. 1A). Meganuclease targeting the NANOG2 sequence is such an enzyme. In that case, no matrix is needed. Some exons can be deleted by the action of one meganuclease (FIGS. 1B and 1C) supplied by a Knock Out DNA matrix. Meganuclesaes recognizing NANOG2 or NANOG4 sequences are useful. A second sub-type of knock-out strategy consists in the replacement of a large region within NANOG gene by the action of two meganucleases (example: NANOG2+NANOG4) and a KO matrix can be used for the deletion of large sequences (FIG. 1D). Such a KO matrix can be built using sequences deleted of the targeted exon as well as some mutated exons.

FIG. 2 a and b illustrate the combinatorial approach, described in International PCT applications WO 2006/097784 and WO 2006/097853 and also in Arnould, et al. (J. Mol. Biol., 2006, 355, 443-458) and Smith et al. (Nucleic Acids Res., 2006). This approach was used to entirely redesign the DNA binding domain of the I-CreI protein and thereby engineer novel meganucleases with fully engineered specificity.

FIG. 3: NANOG2 and NANOG2 derived targets. The NANOG2.1 target sequence (SEQ ID NO: 8) and its derivatives 10AAC_P (SEQ ID NO: 4), 10TAG_P (SEQ ID NO: 6), 5CCT_P (SEQ ID NO: 5) and 5GAG_P (SEQ ID NO: 7), P stands for Palindromic) are derivatives of C1221, found to be cleaved by previously obtained I-CreI mutants. C1221 (SEQ ID NO: 2), 10AAC_P (SEQ ID NO: 4), 10TAG_P (SEQ ID NO: 6), 5CCT_P (SEQ ID NO: 5) and 5GAG_P (SEQ ID NO: 7), were first described as 24 bp sequences, but structural data suggest that only the 22 bp are relevant for protein/DNA interaction. NANOG2.1 (SEQ ID NO: 8) is the DNA sequence located in the human NANOG gene at position 3786-3809. NANOG2.2 (SEQ ID NO: 9) differs from NANOG2.1 at positions −2; −1; +1; +2 where I-CreI cleavage site (GTAC) substitutes the corresponding NANOG2.1 sequence. NANOG2.3 (SEQ ID NO: 10) is the palindromic sequence derived from the left part of NANOG2.2, and NANOG2.4 (SEQ ID NO: 11) is the palindromic sequence derived from the right part of NANOG2.2. NANOG2.5 (SEQ ID NO: 12) is the palindromic sequence derived from the left part of NANOG2.1, and NANOG2.6 (SEQ ID NO: 13) is the palindromic sequence derived from the right part of NANOG2.1.

FIG. 4: Activity cleavage in CHO cells of single chain heterodimer pCLS4412, pCLS4413, pCLS4414, pCLS4415, pCLS4416, pCLS4417, pCLS4418, pCLS4419 compared to ISceI (pCLS1090) and SCOH-RAG-CLS (pCLS2222) meganucleases as positive controls. The empty vector control (pCLS1069) has also been tested on each target. Plasmid pCLS1728 contains control RAG1.10.1 target sequence. In FIG. 6, the correspondence of the line graphs at their right ends to the legend (graph: legend) on the right is as follows: graph 1 (top): 8; 2:5, 3:2, 4:9, 5:6, 6:7, 7:10, 8:4, 9:3, 10:1; 11 (empty vector): 11 (bottom dotted line).

FIG. 5: NANOG4 and NANOG4 derived targets. The NANOG4.1 target sequence (SEQ ID NO: 18) and its derivatives 10TGA_P (SEQ ID NO: 14), 10AAG_P (SEQ ID NO: 16), 5GCT_P (SEQ ID NO: 15) and 5ATT_P (SEQ ID NO: 17), P stands for Palindromic) are derivatives of C1221, found to be cleaved by previously obtained I-CreI mutants. C1221 (SEQ ID NO: 2), 10TGA_P (SEQ ID NO: 14), 10AAG_P (SEQ ID NO: 16), 5GCT_P (SEQ ID NO: 15) and 5ATT_P (SEQ ID NO: 17), were first described as 24 bp sequences, but structural data suggest that only the 22 bp are relevant for protein/DNA interaction. NANOG4.1 (SEQ ID NO: 18) is the DNA sequence located in the human NANOG gene at position 1222-1245. NANOG4.2 (SEQ ID NO: 19) differs from NANOG4.1 at positions −2; −1; +1; +2 where I-CreI cleavage site (GTAC) substitutes the corresponding NANOG4.1 sequence. NANOG4.3 (SEQ ID NO: 20) is the palindromic sequence derived from the left part of NANOG4.2, and NANOG4.4 (SEQ ID NO: 21) is the palindromic sequence derived from the right part of NANOG4.2. NANOG4.5 (SEQ ID NO: 22) is the palindromic sequence derived from the left part of NANOG4.1, and NANOG4.6 (SEQ ID NO: 23) is the palindromic sequence derived from the right part of NANOG4.1.

FIG. 6: Activity cleavage in CHO cells of single chain heterodimer pCLS4420, pCLS4421, pCLS4422, pCLS4697, pCLS4698, pCLS4699, pCLS4701 and pCLS4702 compared to ISceI (pCLS1090) and SCOH-RAG-CLS (pCLS2222) meganucleases as positive controls. The empty vector control (pCLS1069) has also been tested on each target. Plasmid pCLS1728 contains control RAG1.10.1 target sequence. In FIG. 6, the correspondence of the line graphs at their right ends to the legend (graph:legend) on the right is as follows: graph 1 (top): 4; 2:5, 3:8, 4:7, 5:3, 6:2, 7:1, 8:6, 9:10, 10:9; 11 (empty vector): 11 (bottom dotted line).

FIG. 7: Expression profiles of NANOG meganucleases in 293H cells (panel A) and iPS cells (panel B); pCLS2222 corresponding to the RAG1 meganuclease is used as positive control for the experiment. The arrow shows the expression level of the different meganucleases.

FIG. 8: Map of Plasmid pCLS1072.

FIG. 9: Map of Plasmid pCLS1090.

FIG. 10: Map of Plasmid pCLS2222.

FIG. 11: Map of Plasmid pCLS1853.

FIG. 12: Map of Plasmid pCLS1107.

FIG. 13: Map of Plasmid pCLS0002.

FIG. 14: Map of Plasmid pCLS1069.

FIG. 15: Map of Plasmid pCLS1058.

FIG. 16: Map of Plasmid pCLS1728.

FIG. 17: Example of targeted integration identified by PCR screen.

FIG. 18: Example of targeted integration identified by southern blot analysis.

FIG. 19: Example of Pop-out events identified by PCR screen.

FIG. 20: Strategy for NANOG KO using NANOG4 meganucleases. (A) Homology for recombination design; (B) General scheme of matrices; (C) Homologous recombination process mediated by NANOG4 meganucleases.

FIG. 21: Matrices design for irreversible (A), reversible (B), clean reversible (C) NANOG KO.

### DETAILED DESCRIPTION OF THE INVENTION

The present invention concerns a process to generate new class of induced Pluripotent Stem (iPS) cells and their derivatives characterized as clean and/or safe and/or secure by using endonucleases such as meganucleases and particularly the meganucleases of the present invention.

Key issues of current protocols to generate iPS by introducing the four transcription factors Oct3/4, Sox2, KLF4 and c-myc are that:
these introductions are not controlled and lead to heterogenous populations of iPS cells where transgenes are not inserted at the same locus and/or not with the same copy number,
iPS cells express these four transgenes permanently leading to problems for further differentiation steps.
Endonucleases of the present invention are a tool of choice overcoming these classical issues allowing:
stable, robust and single copy targeted insertion of the four transgenes at a defined locus allowing a controlled generation of homogenous iPS populations in high quantity.
the possibility to remove the four transgenes once iPS have been generated without any scar on the genome (“pop-out”), for obtaining clean iPS in further re-differentiation steps and therapeutic uses.

Another issue addressed by endonucleases of the present invention is the possibility to generate secured iPS and to standardize well-defined but still empirical current protocols. By using meganucleases inducing the targeting and the disruption of Nanog or Tert gene as non limiting examples, at a defined step of differentiation process, the progression of iPS toward differentiation states is made irreversible and safe since infinite self-renewable property of these cells is lost.

Also, by using endonucleases to insert at a safe locus of the genome, inducible genes defined as essential for progression of iPS toward differentiated cells (growth factors, transcription factors), it is possible to standardize the differentiation steps of an iPS.

This endonuclease approach of iPS generation and differentiation open new avenues for screening molecules and/or genes in vitro:
in order to securize and standardize the iPS differentiation process, gene candidates from an expression library responsible or implicated in a defined differentiation step can be inserted at a safe locus of an iPS genome locus, by using endonucleases.
to screen chemical libraries for compounds on primary cells carrying or not a genetical defect.
in order to evaluate drug response at a single patient scale in pharmacogenomic approaches.
to confirm or invalidate strategies or chemicals derived from predictive methods and algorithms in predictive toxicology measures.

Also, endonucleases can be the ideal tool to create reporter cell lines integrating at a safe locus, reporter gene fused to a promoter specific of a defined reprogrammation step in order to validate the iPS reprogrammation process. The same approach can be envisioned during the re-differentiation process, allowing to precisely control this process and create progenitor cells bank, still able to divide a limited number of times and known to be able to move through the body and migrate towards the tissue where they are needed; they are particularly useful for adult organisms therapy as they act as a repair system for the body without presenting the known transplantation problem of compatibility.

Regarding NANOG function, the targeting of this gene will be useful to better understand the pluripotency properties of pluripotent stem cells by knock-in and knock-out experiments in ES and iPS cells. For this purpose NANOG recognizing meganucleases are the tool of choice because they can be designed to target specifically this gene. Thus, it will be possible to knock-out the gene specifically but also to knock-in reporter gene which will be expressed under NANOG regulators element. Thus, NANOG expression could be followed both at the undifferentiated and differentiated stages. Such approach will also allow to monitor the process of de-differentiation of differentiated cells.

Another application of NANOG designed meganucleases will be for the study of the reprogramming process and for the identification of new factors able to play a role in this process. In fact, although huge work has been made by the scientific community, the reprogramming process remains still largely inefficient (<0.1%) and not well controlled. Moreover strategy based on transgene integration are presently the most efficient, but they suffer major drawbacks. The integration site for transgenesis remains unpredictable and irreproducible, which can affect endogenous cellular gene functions or promote tumorigenesis. In addition, although integrated reprogramming factors become transcriptionally silenced over time through de novo DNA methylation, they can be spontaneously reactivated during cell culture and differentiation. The development of new strategy to improve the reprogramming process is therefore required.

Taking advantage of NANOG meganucleases, it will be possible to knock-in into somatic cells a reporter gene under the control of the endogenous NANOG regulatory sequences and control elements to monitor reprogramming efficiency through the expression of the reporter gene that will mimic the activation of the pluripotency gene NANOG.

Finally, NANOG meganucleases could be also useful to reduce the tumorigenic potential of pluripotent stem cells by knocking down this gene. In fact, recent work on ES cells has highlighted the presence of abnormal overgrowth after engraftment into animals of differentiated precursors derived from ES cells (Tabar et al, 2005, Roy et al, 2006, Aubry et al, 2008). Choice of NANOG as a candidate for this purpose is also based on the fact that recently NANOG has been described for its potential role in human tumor development (Jeter et al, 2009; You et al, 2009; Ji et al, 2009). In this context, the knock-out of hNANOG inhibits tumor formation by reducing proliferation and clonogenic growth. Pluripotent stem cells are useful for cell therapy (Brignier at al, The Journal of Allergy Clinical Immunology) and drug screening (Phillips et al, Biodrugs 2010) because they give access to all cell types of the body as neurons for example. They have also a human origin; they can be obtained in unlimited quantities. In fact, cell therapy or drug screening studies are performed using primary cells which are obtained in limited quantities and have few proliferative potential. Another source is adult stem cells but compared to pluripotent stem cells they are still limited due to their access and their culture conditions. Moreover, regarding transplantation, problem of compatibility are still present; this problem could be overcome using iPS cells which can be derived directly from the patient to graft.

For drug screening studies iPS cells are valuable since for a given disease, iPS cells could be generated for several patients and their unaffected parents, given thus access to the human diversity. Moreover, the mutation causal of the pathology is not induced is the original one. Then the effect of the mutation can be studied in different tissues to identify the effect of a potential drug on the affected tissue but also on others tissues to check the absence of secondary effects.

Meganucleases directed against NANOG will therefore represent a tool of choice for several applications which will permit to better understand pluripotent stem cells and thus may be overcome actual problems lead by these cells for cell therapy and drug screening studies.

As mentioned above certain aspects of the invention reflect different strategies for modulating, modifying or controlling NANOG gene expression that can be implemented with the NANOG recognizing meganucleases of the invention. In more detail these include:

Meganucleases that Recognize NANOG Target Sequences

Table I below shows target nucleotide sequences within the NANOG locus recognized by meganucleases of the invention. Target sites inside (NANOG2) and outside (NANOG4) of the NANOG coding sequence are useful for different procedures. For example, insertion into NANOG2 is useful in producing knock out mutations of NANOG and insertion into NANOG4 can be used to introduce regulatory or reporter sequences.

TABLE I
sequences and location of the targeted sites in the
NANOG gene

| | | | SEQ ID |
|---|---|---|---|
| Target | location | Sequence | NO: |
| NANOG1 | 3576 within | ATCTGCTTATTCAGGACAGCCCTG | 66 |
| | exon 2 | | |
| NANOG2 | 3786 within | CCAACATCCTGAACCTCAGCTACA | 8 |
| | exon 2 | | |
| NANOG3 | 5500 within | TATAACTGTGGAGAGGAATCTCTG | 67 |
| | exon 4 | | |
| NANOG4 | 1222 within | ACTGAACGCTGTAAAATAGCTTAA | 18 |
| | intron 1 | | |
| NANOG5 | 3991 within | ATTCTATTATGTGAATAATTATGT | 68 |
| | intron 2 | | |
| NANOG6 | 3919 within | ATCGCCTCTTGCAAATAATTTATG | 69 |
| | intron 2 | | |
| NANOG7 | 5028 within | ATTTTACAATTTCTATCATTTTTT | 70 |
| | intron 2 | | |
| NANOG8 | 6500 after | CTAATCTTTGTAGAAAGAGGTCTC | 71 |
| | exon 4 | | |

Endonucleases that Recognize NANOG Target Sequences

Table Ibis below shows target nucleotide sequences within the NANOG locus recognized by endonucleases of the invention.

TABLE Ibis
sequences of targeted sites in the NANOG gene

| | | | SEQ ID |
|---|---|---|---|
| Target | Location | Sequences | NO: |
| 2 | exon1 | TGTGGATCCAGCTTGTCCCCAAAGCTTGCCTTGCTTTGAAGCATCCGACTGTAAAGAATCTTCA | 72 |
| 3 | exon1 | TCCAGCTTGTCCCCAAAGCTTGCCTTGCTTTGAAGCATCCGACTGTAAAGAATCTTCACCTA | 73 |
| 4 | exon1 | TTGCTTTGAAGCATCCGACTGTAAAGAATCTTCACCTATGCCTGTGATTTGTGGGCCTGAAGAAAACTA | 74 |
| 6 | exon1 | TAAAGAATCTTCACCTATGCCTGTGATTTGTGGGCCTGAAGAAAACTATCCATCCTTGCAAA | 75 |
| 7 | exon1 | TGGGCCTGAAGAAAACTATCCATCCTTGCAAATGTCTTCTGCTGAGATGCCTCACACGGAGA | 76 |
| 9 | exon2 | TGGATCTGCTTATTCAGGACAGCCCTGATTCTTCCACCAGTCCCAAAGGCAAACAACCCA | 77 |
| 15 | exon3 | TGGTTCCAGAACCAGAGAATGAAATCTAAGAGGTGGCAGAAAAACAACTGGCCGAAGAATAGCAA | 78 |
| 17 | exon4 | TTTACTCTTCCTACCACCAGGGATGCCTGGTGAACCCGACTGGGAACCTTCCAATGTGGAGCAACCA | 79 |
| 18 | exon4 | TCTTCCTACCACCAGGGATGCCTGGTGAACCCGACTGGGAACCTTCCAATGTGGAGCAACCAGACCTGGAA | 80 |
| 20 | exon4 | TTCCAATGTGGAGCAACCAGACCTGGAACAATTCAACCTGGAGCAACCAGACCCAGAACATCCA | 81 |
| 21 | exon4 | TCCAGTCCTGGAGCAACCACTCCTGGAACACTCAGACCTGGTGCACCCAATCCTGGAACAATCA | 82 |
| 24 | exon4 | TGCCAGTGACTTGGAGGCTGCCTTGGAAGCTGCTGGGGAAGGCCTTAATGTAATACAGCAGA | 83 |

Methods for Knocking-Out (KO) NANOG Gene Expression

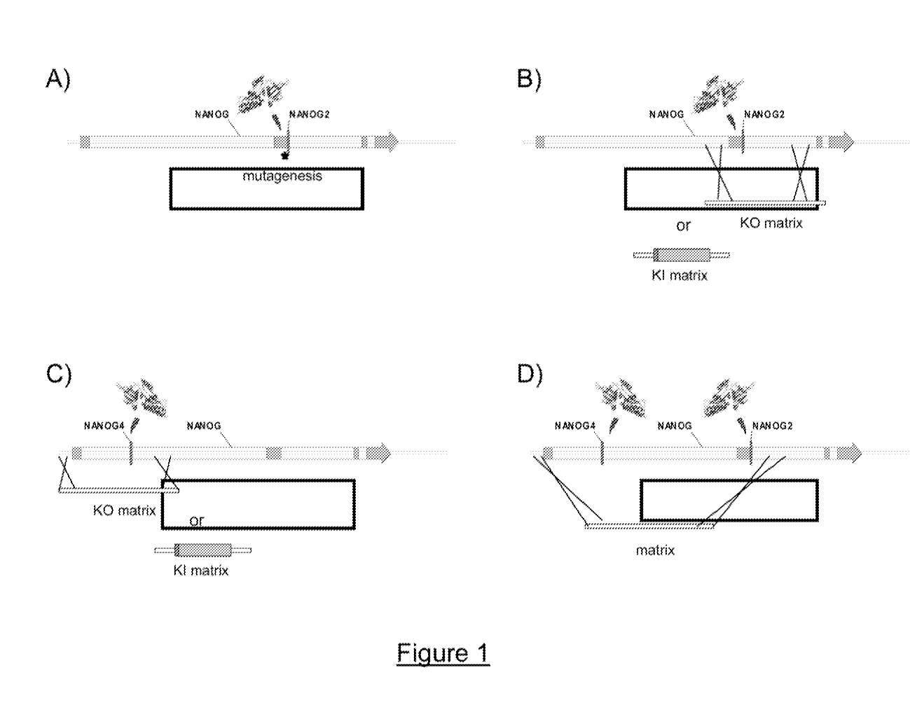

Different strategies can be implemented for knocking out the NANOG (FIG. 1). The coding sequence can be mutated by non homologous end joining (NHEJ) using a meganuclease targeting a sequence in the open reading frame (FIG. 1A). Meganuclease targeting the NANOG2 sequence is such an enzyme. In that case, no matrix is needed. Some exons can be deleted by the action of one meganuclease (FIGS. 1B and 1C) supplied by a Knocking Out DNA matrix. Meganuclesaes recognizing NANOG2 or NANOG4 sequences are useful. A second sub-type of knock-out strategy consists in the replacement of a large region within NANOG gene by the action of two meganucleases (example: NANOG2+NANOG4) and a KO matrix can be used for the deletion of large sequences (FIG. 1). Such a KO matrix can be built using sequences deleted of the targeted exon as well as some mutated exons.

Knocking In (“KI”) a Gene of Interest KI at the NANOG Locus

Since the NANOG locus can be used for the expression of reporter and genes of interest, some meganuclease targeting sequences in exons (FIG. 1B) or in introns (FIG. 1C) are useful for the integration of knock in matrix by homologous recombination. Such a KI matrix can be built using sequences homologous to the targeted locus added of the gene of interest with or without regulation elements.

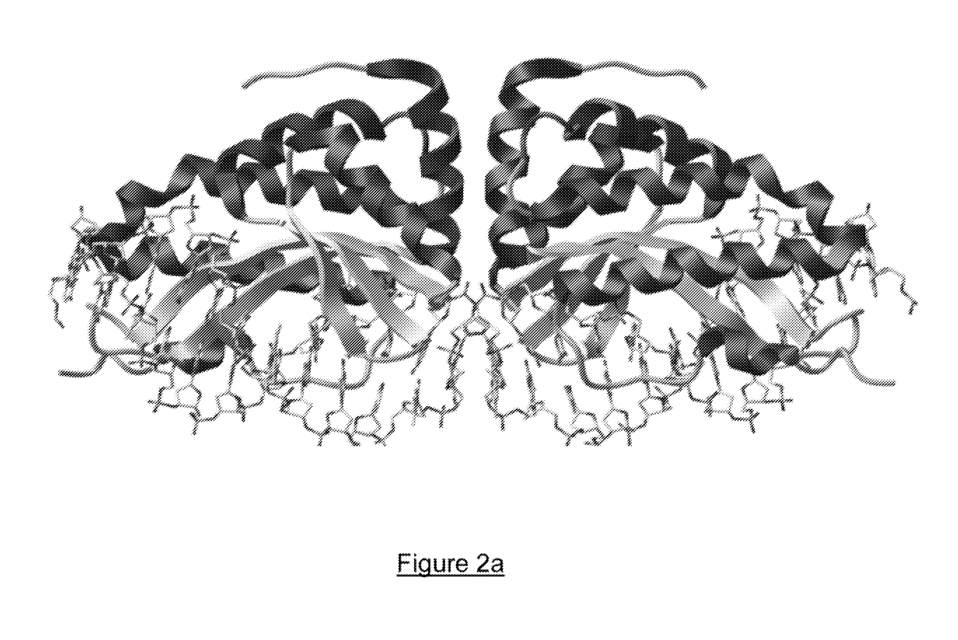

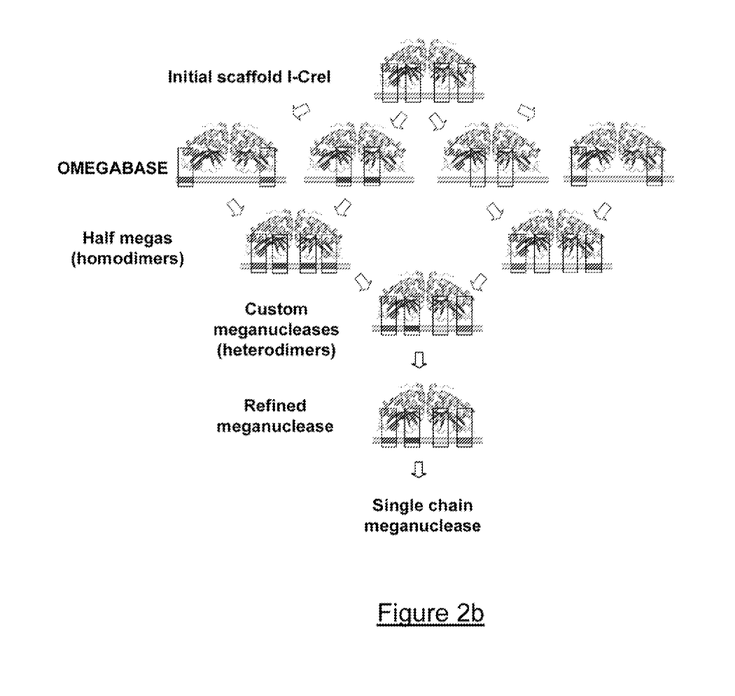

I-CreI variants of the present invention were created using the combinatorial approach illustrated in FIG. 2b and described in International PCT applications WO 2006/097784 and WO 2006/097853, and also in Arnould et al. (J. Mol. Biol., 2006, 355, 443-458) and Smith et al. (Nucleic Acids Res., 2006), allowing to redesign the DNA binding domain of the I-CreI protein and thereby engineer novel meganucleases with fully engineered specificity.

The cleavage activity of the variant according to the invention may be performed by any well-known, in vitro or in vivo cleavage assay, such as those described in the International PCT Application WO 2004/067736; Epinat et al., Nucleic Acids Res., 2003, 31, 2952-2962; Chames et al., Nucleic Acids Res., 2005, 33, e178; Arnould et al., J. Mol. Biol., 2006, 355, 443-458, and Arnould et al., J. Mol. Biol., 2007, 371, 49-65. For example, the cleavage activity of the variant of the invention may be measured by a direct repeat recombination assay, in yeast or mammalian cells, using a reporter vector. The reporter vector comprises two truncated, non-functional copies of a reporter gene (direct repeats) and the genomic (non-palindromic) DNA target sequence within the intervening sequence, cloned in yeast or in a mammalian expression vector. Usually, the genomic DNA target sequence comprises one different half of each (palindromic or pseudo-palindromic) parent homodimeric I-CreI meganuclease target sequence. Expression of the heterodimeric variant results in a functional endonuclease which is able to cleave the genomic DNA target sequence. This cleavage induces homologous recombination between the direct repeats, resulting in a functional reporter gene, whose expression can be monitored by an appropriate assay. The cleavage activity of the variant against the genomic DNA target may be compared to wild type I-CreI or I-SceI activity against their natural target.

Possibly or not, at least two rounds of selection/screening are performed according to the process illustrated Arnould et al., J. Mol. Biol., 2007, 371, 49-65. In the first round, one of the monomers of the heterodimer is mutagenised, co-expressed with the other monomer to form heterodimers, and the improved monomers Y⁺ are selected against the target from the gene of interest. In the second round, the other monomer (monomer X) is mutagenised, co-expressed with the improved monomers Y⁺ to form heterodimers, and selected against the target from the gene of interest to obtain meganucleases (X⁺ Y⁺) with improved activity. The mutagenesis may be random-mutagenesis or site-directed mutagenesis on a monomer or on a pool of monomers, as indicated above. Both types of mutagenesis are advantageously combined. Additional rounds of selection/screening on one or both monomers may be performed to improve the cleavage activity of the variant.

In a preferred embodiment of said variant, said substitution(s) in the subdomain situated from positions 44 to 77 of I-CreI are at positions 44, 68, 70, 75 and/or 77.

In another preferred embodiment of said variant, said substitution(s) in the subdomain situated from positions 28 to 40 of I-CreI are at positions 28, 30, 32, 33, 38 and/or 40.

In another preferred embodiment of said variant, it comprises one or more mutations in I-CreI monomer(s) at positions of other amino acid residues that contact the DNA target sequence or interact with the DNA backbone or with the nucleotide bases, directly or via a water molecule; these residues are well-known in the art (Jurica et al., Molecular Cell, 1998, 2, 469-476; Chevalier et al., J. Mol. Biol., 2003, 329, 253-269). In particular, additional substitutions may be introduced at positions contacting the phosphate backbone, for example in the final C-terminal loop (positions 137 to 143; Prieto et al., Nucleic Acids Res., Epub 22 Apr. 2007).

Preferably said residues are involved in binding and cleavage of said DNA cleavage site.

More preferably, said residues are at positions 138, 139, 142 or 143 of I-CreI. Two residues may be mutated in one variant provided that each mutation is in a different pair of residues chosen from the pair of residues at positions 138 and 139 and the pair of residues at positions 142 and 143. The mutations which are introduced modify the interaction(s) of said amino acid(s) of the final C-terminal loop with the phosphate backbone of the I-CreI site. Preferably, the residue at position 138 or 139 is substituted by a hydrophobic amino acid to avoid the formation of hydrogen bonds with the phosphate backbone of the DNA cleavage site. For example, the residue at position 138 is substituted by an alanine or the residue at position 139 is substituted by a methionine. The residue at position 142 or 143 is advantageously substituted by a small amino acid, for example a glycine, to decrease the size of the side chains of these amino acid residues.

More preferably, said substitution in the final C-terminal loop modify the specificity of the variant towards the nucleotide at positions ±1 to 2, ±6 to 7 and/or ±11 to 12 of the I-CreI site.

In another preferred embodiment of said variant, it comprises one or more additional mutations that improve the binding and/or the cleavage properties of the variant towards the DNA target sequence from the NANOG gene. The additional residues which are mutated may be on the entire I-CreI sequence, and in particular in the C-terminal half of I-CreI (positions 80 to 163). Both I-CreI monomers are advantageously mutated; the mutation(s) in each monomer may be identical or different. For example, the variant comprises one or more additional substitutions at positions: 2, 7, 8, 19, 43, 54, 61, 80, 81, 96, 105 and 132. Said substitutions are advantageously selected from the group consisting of: N2S, K7E, E8K, G19S, F43L, F54L, E61R, E80K, I81T, K96E, V105A and I132V. More preferably, the variant comprises at least one substitution selected from the group consisting of: N2S, K7E, E8K, G19S, F43L, F54L, E61R, E80K, I81T, K96E, V105A and I132V. The variant may also comprise additional residues at the C-terminus. For example a glycine (G) and/or a proline (P) residue may be inserted at positions 164 and 165 of I-CreI, respectively.

According to a preferred embodiment, said additional mutation in said variant further impairs the formation of a functional homodimer. More preferably, said mutation is the G19S mutation. The G19S mutation is advantageously introduced in one of the two monomers of a heterodimeric I-CreI variant, so as to obtain a meganuclease having enhanced cleavage activity and enhanced cleavage specificity. In addition, to enhance the cleavage specificity further, the other monomer may carry a distinct mutation that impairs the formation of a functional homodimer or favors the formation of the heterodimer.

In another preferred embodiment of said variant, said substitutions are replacement of the initial amino acids with amino acids selected from the group consisting of: A, D, E, G, H, K, N, P, Q, R, S, T, Y, C, V, L, M, F, I and W.

In particular the variant is selected from the group consisting of SEQ ID NO: 25 to 32 and 33 to 40.

The variant of the invention may be derived from the wild-type I-CreI (SEQ ID NO: 1) or an I-CreI scaffold protein having at least 85% identity, preferably at least 90% identity, more preferably at least 95% identity with SEQ ID NO: 1, such as the scaffold called I-CreI N75 (167 amino acids; SEQ ID NO: 2) having the insertion of an alanine at position 2, and the insertion of AAD at the C-terminus (positions 164 to 166) of the I-CreI sequence. In the present patent application all the I-CreI variants described comprise an additional Alanine after the first Methionine of the wild type I-CreI sequence (SEQ ID NO: 1). These variants also comprise two additional Alanine residues and an Aspartic Acid residue after the final Proline of the wild type I-CreI sequence. These additional residues do not affect the properties of the enzyme and to avoid confusion these additional residues do not affect the numeration of the residues in I-CreI or a variant referred in the present patent application, as these references exclusively refer to residues of the wild type I-CreI enzyme (SEQ ID NO: 1) as present in the variant, so for instance residue 2 of I-CreI is in fact residue 3 of a variant which comprises an additional Alanine after the first Methionine.

In addition, the variants of the invention may include one or more residues inserted at the NH₂ terminus and/or COOH terminus of the sequence. For example, a tag (epitope or polyhistidine sequence) is introduced at the NH₂ terminus and/or COOH terminus; said tag is useful for the detection and/or the purification of said variant. The variant may also comprise a nuclear localization signal (NLS); said NLS is useful for the importation of said variant into the cell nucleus. The NLS may be inserted just after the first methionine of the variant or just after an N-terminal tag.

The variant according to the present invention may be a homodimer which is able to cleave a palindromic or pseudo-palindromic DNA target sequence.

Alternatively, said variant is a heterodimer, resulting from the association of a first and a second monomer having different substitutions at positions 28 to 40 and 44 to 77 of I-CreI, said heterodimer being able to cleave a non-palindromic DNA target sequence from the NANOG gene.

In particular said heterodimer variant is composed by one of the possible associations between variants constituting N-terminal and C-terminal monomers of single chain molecules from the group consisting of SEQ ID NO: 25 to SEQ ID NO: 32 and SEQ ID NO: 33 to SEQ ID NO: 40.

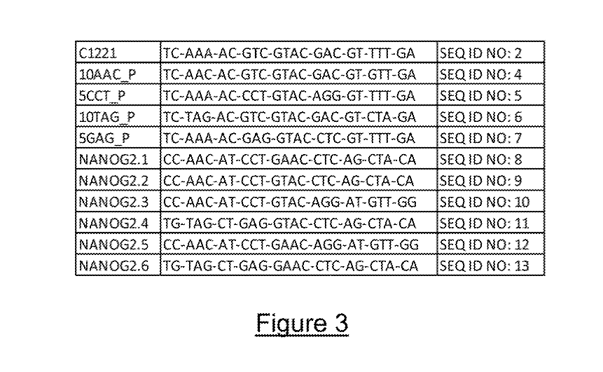

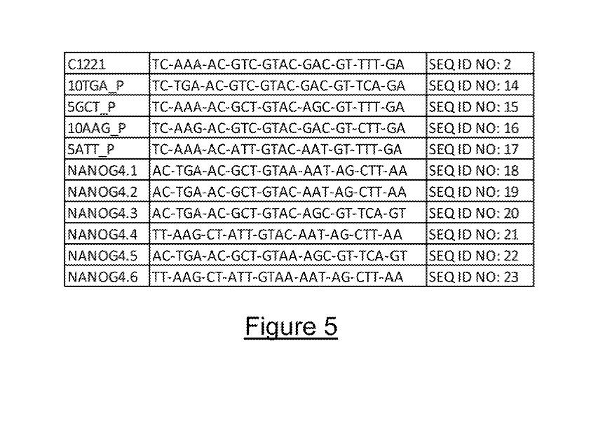

The DNA target sequences are situated in the NANOG Open Reading Frame (ORF) and these sequences cover all the NANOG ORF. In particular, said DNA target sequences for the variant of the present invention and derivatives are selected from the group consisting of the SEQ ID NO: 4 to SEQ ID NO: 23, as shown in FIGS. 3 and 5 and Table I.

The sequence of each I-CreI variant is defined by the mutated residues at the indicated positions. The positions are indicated by reference to I-CreI sequence (SEQ ID NO: 1); I-CreI has N, S, Y, Q, S, Q, R, R, D, I and E at positions 30, 32, 33, 38, 40, 44, 68, 70, 75, 77 and 80 respectively.

Each monomer (first monomer and second monomer) of the heterodimeric variant according to the present invention may also be named with a letter code, after the eleven residues at positions 28, 30, 32, 33, 38, 40, 44, 68 and 70, 75 and 77 and the additional residues which are mutated, as indicated above. For example, the mutations 7E28R33R38Y40Q44K54164A68A70G75N96E147A in the N-terminal monomer constituting a single chain molecule targeting the NANOG2 target of the present invention (SEQ ID NO: 46).

In the present invention, for a given DNA target, “0.2” derivative target sequence differs from the initial genomic target at positions −2, −1, +1, +2, where I-CreI cleavage site (GTAC) substitutes the corresponding sequence at these positions of said initial genomic target. “0.3” derivative target sequence is the palindromic sequence derived from the left part of said “0.2” derivative target sequence. “0.4” derivative target sequence is the palindromic sequence derived from the right part of said “0.2” derivative target sequence. “0.5” derivative target sequence is the palindromic sequence derived from the left part of the initial genomic target. “0.6” derivative is the palindromic sequence derived from the left part of the initial genomic target.

In the present invention, a “N-terminal monomer” constituting one of the monomers of a single chain molecule, refers to a variant able to cleave “0.3” or “0.5” palindromic sequence. In the present invention, a “C-terminal monomer” constituting one of the monomers of a single chain molecule, refers to a variant able to cleave “0.4” or “0.6” palindromic sequence.

The heterodimeric variant as defined above may have only the amino acid substitutions as indicated above. In this case, the positions which are not indicated are not mutated and thus correspond to the wild-type I-CreI (SEQ ID NO: 1).

The invention encompasses I-CreI variants having at least 85% identity, preferably at least 90% identity, more preferably at least 95% (96%, 97%, 98%, 99%) identity with the sequences as defined above, said variant being able to cleave a DNA target from the NANOG gene.

The heterodimeric variant is advantageously an obligate heterodimer variant having at least one pair of mutations corresponding to residues of the first and the second monomers which make an intermolecular interaction between the two I-CreI monomers, wherein the first mutation of said pair(s) is in the first monomer and the second mutation of said pair(s) is in the second monomer and said pair(s) of mutations prevent the formation of functional homodimers from each monomer and allow the formation of a functional heterodimer, able to cleave the genomic DNA target from the NANOG gene.

To form an obligate heterodimer, the monomers have advantageously at least one of the following pairs of mutations, respectively for the first monomer and the second monomer:

a) the substitution of the glutamic acid at position 8 with a basic amino acid, preferably an arginine (first monomer) and the substitution of the lysine at position 7 with an acidic amino acid, preferably a glutamic acid (second monomer); the first monomer may further comprise the substitution of at least one of the lysine residues at positions 7 and 96, by an arginine,

b) the substitution of the glutamic acid at position 61 with a basic amino acid, preferably an arginine (first monomer) and the substitution of the lysine at position 96 with an acidic amino acid, preferably a glutamic acid (second monomer); the first monomer may further comprise the substitution of at least one of the lysine residues at positions 7 and 96, by an arginine,

c) the substitution of the leucine at position 97 with an aromatic amino acid, preferably a phenylalanine (first monomer) and the substitution of the phenylalanine at position 54 with a small amino acid, preferably a glycine (second monomer); the first monomer may further comprise the substitution of the phenylalanine at position 54 by a tryptophane and the second monomer may further comprise the substitution of the leucine at position 58 or lysine at position 57, by a methionine, and

d) the substitution of the aspartic acid at position 137 with a basic amino acid, preferably an arginine (first monomer) and the substitution of the arginine at position 51 with an acidic amino acid, preferably a glutamic acid (second monomer).

For example, the first monomer may have the mutation D137R and the second monomer, the mutation R51D. The obligate heterodimer meganuclease comprises advantageously, at least two pairs of mutations as defined in a), b), c) or d), above; one of the pairs of mutation is advantageously as defined in c) or d). Preferably, one monomer comprises the substitution of the lysine residues at positions 7 and 96 by an acidic amino acid (aspartic acid (D) or glutamic acid (E)), preferably a glutamic acid (K7E and K96E) and the other monomer comprises the substitution of the glutamic acid residues at positions 8 and 61 by a basic amino acid (arginine (R) or lysine (K); for example, E8K and E61R). More preferably, the obligate heterodimer meganuclease, comprises three pairs of mutations as defined in a), b) and c), above.

The obligate heterodimer meganuclease consists advantageously of a first monomer (A) having at least the mutations (i) E8R, E8K or E8H, E61R, E61K or E61H and L97F, L97W or L97Y; (ii) K7R, E8R, E61R, K96R and L97F, or (iii) K7R, E8R, F54W, E61R, K96R and L97F and a second monomer (B) having at least the mutations (iv) K7E or K7D, F54G or F54A and K96D or K96E; (v) K7E, F54G, L58M and K96E, or (vi) K7E, F54G, K57M and K96E. For example, the first monomer may have the mutations K7R, E8R or E8K, E61R, K96R and L97F or K7R, E8R or E8K, F54W, E61R, K96R and L97F and the second monomer, the mutations K7E, F54G, L58M and K96E or K7E, F54G, K57M and K96E. The obligate heterodimer may comprise at least one NLS and/or one tag as defined above; said NLS and/or tag may be in the first and/or the second monomer.

The subject-matter of the present invention is also a single-chain chimeric meganuclease (fusion protein) derived from an I-CreI variant as defined above. The single-chain meganuclease may comprise two I-CreI monomers, two I-CreI core domains (positions 6 to 94 of I-CreI) or a combination of both. Preferably, the two monomers/core domains or the combination of both, are connected by a peptidic linker.

More preferably the single-chain chimeric meganuclease is composed by one of the possible associations between variants from the group consisting of N-terminal monomers and C-terminal monomers, given in Tables II and III, respectively for a given DNA target, at the NANOG2 and NANOG4 loci, said monomer variants being connected by a linker. More preferably the single-chain chimeric meganuclease according to the present invention is one from the group consisting of SEQ ID NO: 25 to SEQ ID NO: 32 and SEQ ID NO: 33 to SEQ ID NO: 40. Regarding NANOG2.1 target at NANOG2 locus, the single-chain chimeric meganuclease according to the present invention is one from the group consisting of SEQ ID NO: 25 to SEQ ID NO: 32. Regarding NANOG4.1 target, the single-chain chimeric meganuclease according to the present invention is one from the group consisting of SEQ ID NO: 33 to SEQ ID NO: 40.

It is understood that the scope of the present invention also encompasses the I-CreI variants per se, including heterodimers, obligate heterodimers, single chain meganucleases as non limiting examples, able to cleave one of the target sequences in NANOG gene.

It is also understood that the scope of the present invention also encompasses the I-CreI variants as defined above that target equivalent sequences in NANOG gene of eukaryotic organisms other than human, preferably mammals, more preferably a laboratory rodent (mice, rat, guinea-pig), or a rabbit, a cow, pig, horse or goat, those sequences being identified by the man skilled in the art in public databank like NCBI.

The subject-matter of the present invention is also a polynucleotide fragment encoding a variant or a single-chain chimeric meganuclease as defined above; said polynucleotide may encode one monomer of a homodimeric or heterodimeric variant, or two domains/monomers of a single-chain chimeric meganuclease. It is understood that the subject-matter of the present invention is also a polynucleotide fragment encoding one of the variant species as defined above, obtained by any method well-known in the art.

The subject-matter of the present invention is also a recombinant vector for the expression of a variant or a single-chain meganuclease according to the invention. The recombinant vector comprises at least one polynucleotide fragment encoding a variant or a single-chain meganuclease, as defined above. In a preferred embodiment, said vector comprises two different polynucleotide fragments, each encoding one of the monomers of a heterodimeric variant.

A vector which can be used in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those skilled in the art and commercially available.

Viral vectors include retrovirus, adenovirus, parvovirus (e.g. adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus (particularly self inactivating lentiviral vectors), spumavirus (Coffin, J. M., Retroviridae: The viruses and their replication, In Fundamental Virology, Third Edition, B. N. Fields, et al., Eds., Lippincott-Raven Publishers, Philadelphia, 1996).

Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, Glutamine Synthetase, and hypoxanthine-guanine phosphoribosyl transferase for eukaryotic cell culture; TRP1, URA3 and LEU2 for S. cerevisiae; tetracycline, rifampicin or ampicillin resistance in E. coli.

Preferably said vectors are expression vectors, wherein the sequence(s) encoding the variant/single-chain meganuclease of the invention is placed under control of appropriate transcriptional and translational control elements to permit production or synthesis of said variant. Therefore, said polynucleotide is comprised in an expression cassette. More particularly, the vector comprises a replication origin, a promoter operatively linked to said polynucleotide, a ribosome-binding site, an RNA-splicing site (when genomic DNA is used), a polyadenylation site and a transcription termination site. It also can comprise an enhancer. Selection of the promoter will depend upon the cell in which the polypeptide is expressed. Preferably, when said variant is a heterodimer, the two polynucleotides encoding each of the monomers are included in one vector which is able to drive the expression of both polynucleotides, simultaneously. Suitable promoters include tissue specific and/or inducible promoters. Examples of inducible promoters are: eukaryotic metallothionine promoter which is induced by increased levels of heavy metals, prokaryotic lacZ promoter which is induced in response to isopropyl-β-D-thiogalacto-pyranoside (IPTG) and eukaryotic heat shock promoter which is induced by increased temperature. Examples of tissue specific promoters are skeletal muscle creatine kinase, prostate-specific antigen (PSA), α-antitrypsin protease, human surfactant (SP) A and B proteins, β-casein and acidic whey protein genes.

According to another advantageous embodiment of said vector, it includes a targeting construct comprising sequences sharing homologies with the region surrounding the genomic DNA cleavage site as defined above.

For instance, said sequence sharing homologies with the regions surrounding the genomic DNA cleavage site of the variant is a fragment of the NANOG gene. Alternatively, the vector coding for an I-CreI variant/single-chain meganuclease and the vector comprising the targeting construct are different vectors.

More preferably, the targeting DNA construct comprises:

a) sequences sharing homologies with the region surrounding the genomic DNA cleavage site as defined above, and

b) a sequence to be introduced flanked by sequences as in a) or included in sequences as in a).

Preferably, homologous sequences of at least 50 bp, preferably more than 100 bp and more preferably more than 200 bp are used. Therefore, the targeting DNA construct is preferably from 200 bp to 6000 bp, more preferably from 1000 bp to 2000 bp. Indeed, shared DNA homologies are located in regions flanking upstream and downstream the site of the break and the DNA sequence to be introduced should be located between the two arms. The sequence to be introduced may be any sequence used to alter the chromosomal DNA in some specific way including a sequence used to repair a mutation in the NANOG gene, restore a functional NANOG gene in place of a mutated one, modify a specific sequence in the NANOG gene, to attenuate or activate the NANOG gene, to inactivate or delete the NANOG gene or part thereof, to introduce a mutation into a site of interest or to introduce an exogenous gene or part thereof. Such chromosomal DNA alterations are used for genome engineering (animal models/recombinant cell lines) or genome therapy (gene correction or recovery of a functional gene). The targeting construct comprises advantageously a positive selection marker between the two homology arms and eventually a negative selection marker upstream of the first homology arm or downstream of the second homology arm. The marker(s) allow(s) the selection of cells having inserted the sequence of interest by homologous recombination at the target site.

The sequence to be introduced is a sequence which repairs a mutation in the NANOG gene (gene correction or recovery of a functional gene), for the purpose of genome therapy. For correcting the NANOG gene, cleavage of the gene occurs in the vicinity of the mutation, preferably, within 500 bp of the mutation. The targeting construct comprises a NANOG gene fragment which has at least 200 bp of homologous sequence flanking the target site (minimal repair matrix) for repairing the cleavage, and includes a sequence encoding a portion of wild-type NANOG gene corresponding to the region of the mutation for repairing the mutation. Consequently, the targeting construct for gene correction comprises or consists of the minimal repair matrix; it is preferably from 200 bp to 6000 bp, more preferably from 1000 bp to 2000 bp. Preferably, when the cleavage site of the variant overlaps with the mutation the repair matrix includes a modified cleavage site that is not cleaved by the variant which is used to induce said cleavage in the NANOG gene and a sequence encoding wild-type NANOG gene that does not change the open reading frame of the NANOG gene.

Alternatively, for the generation of knock-in cells/animals, the targeting DNA construct may comprise flanking regions corresponding to NANOG gene fragments which has at least 200 bp of homologous sequence flanking the target site of the I-CreI variant for repairing the cleavage, an exogenous gene of interest within an expression cassette and eventually a selection marker such as the neomycin resistance gene.

For the insertion of a sequence, DNA homologies are generally located in regions directly upstream and downstream to the site of the break (sequences immediately adjacent to the break; minimal repair matrix). However, when the insertion is associated with a deletion of ORF sequences flanking the cleavage site, shared DNA homologies are located in regions upstream and downstream the region of the deletion.

Alternatively, for restoring a functional gene cleavage of the gene occurs in the vicinity or upstream of a mutation. Preferably said mutation is the first known mutation in the sequence of the gene, so that all the downstream mutations of the gene can be corrected simultaneously. The targeting construct comprises the exons downstream of the cleavage site fused in frame (as in the cDNA) and with a polyadenylation site to stop transcription in 3′. The sequence to be introduced (exon knock-in construct) is flanked by introns or exons sequences surrounding the cleavage site, so as to allow the transcription of the engineered gene (exon knock-in gene) into a mRNA able to code for a functional protein. For example, the exon knock-in construct is flanked by sequences upstream and downstream of the cleavage site, from a minimal repair matrix as defined above.

The subject matter of the present invention is also a targeting DNA construct as defined above.

The subject-matter of the present invention is also a composition characterized in that it comprises at least one meganuclease as defined above (variant or single-chain chimeric meganuclease) and/or at least one expression vector encoding said meganuclease, as defined above. Preferably, said composition is a pharmaceutical composition.

In a preferred embodiment of said composition, it comprises a targeting DNA construct, as defined above. Preferably, said targeting DNA construct is either included in a recombinant vector or it is included in an expression vector comprising the polynucleotide(s) encoding the meganuclease according to the invention.

The subject-matter of the present invention is further the use of a meganuclease as defined above, one or two polynucleotide(s), preferably included in expression vector(s), for repairing mutations of the NANOG gene.

The subject-matter of the present invention is also further a method of treatment of a genetic disease caused by a mutation in NANOG gene comprising administering to a subject in need thereof an effective amount of at least one variant encompassed in the present invention.

According to an advantageous embodiment of said use, it is for inducing a double-strand break in a site of interest of the NANOG gene comprising a genomic DNA target sequence, thereby inducing a DNA recombination event, a DNA loss or cell death.

According to the invention, said double-strand break is for: repairing a specific sequence in the NANOG gene, modifying a specific sequence in the NANOG gene, restoring a functional NANOG gene in place of a mutated one, attenuating or activating the NANOG gene, introducing a mutation into a site of interest of the NANOG gene, introducing an exogenous gene or a part thereof, inactivating or deleting the NANOG gene or a part thereof, translocating a chromosomal arm, or leaving the DNA unrepaired and degraded.

Given the fact that NANOG gene is only expressed in iPS cells or cancer cells, therefore, one can consider the NANOG locus as a safe harbor in cells that do not normally express NANOG, provided the insert can be expressed from this locus. In cells that do normally express NANOG, provided the insertion does not affect the expression of NANOG, or provided there remain a functional allele in the cell. For example insertion in introns can be made with no or minor modification of the expression pattern.
However, in this approach, the NANOG gene itself can be disrupted.

Therefore, in another aspect of the present invention, the inventors have found that endonucleases variants targeting NANOG gene can be used for inserting therapeutic transgenes other than NANOG at NANOG gene locus, using this locus as a safe harbor locus. In other terms, the invention relates to a mutant endonuclease capable of cleaving a target sequence in NANOG gene locus, for use in safely inserting a transgene, wherein said disruption or deletion of said locus does not modify expression of genes located outside of said locus.

The subject-matter of the present invention is also further a method of treatment of a genetic disease caused by a mutation in a gene other than NANOG gene comprising administering to a subject in need thereof an effective amount of at least one variant encompassed in the present invention.

The skilled in the art can easily verify whether disruption or deletion of a locus modifies expression of neighboring genes located outside of said locus using proteomic tools. Many protein expression profiling arrays suitable for such an analysis are commercially available. By “neighboring genes” is meant the 1, 2, 5, 10, 20 or 30 genes that are located at each end of the NANOG gene locus.

In a derived main aspect of the present invention, the inventors have found that the NANOG locus could be used as a landing pad to insert and express genes of interest (GOIs) other than therapeutics. In this aspect, inventors have found that genetic constructs containing a GOI could be integrated into the genome at the NANOG gene locus via meganuclease-induced recombination by specific meganuclease variants targeting NANOG gene locus according to a previous aspect of the invention.

The subject-matter of the present invention is also further a method for inserting a transgene into the genomic NANOG locus of a cell, tissue or non-human animal wherein at least one variant of the invention is introduced in said cell, tissue or non-human animal.

In a preferred embodiment, the NANOG locus further allows stable expression of the transgene. In another preferred embodiment, the target sequence inside the NANOG locus is only present once within the genome of said cell, tissue or individual.

In another preferred embodiment meganuclease variants according to the present invention can be part of a kit to introduce a sequence encoding a GOI into at least one cell. In a more preferred embodiment, the at least one cell is selected form the group comprising: CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells.

The subject-matter of the present invention is also a method for making a NANOG gene knock-out or knock-in recombinant cell, comprising at least the step of:

(a) introducing into a cell, a meganuclease as defined above (I-CreI variant or single-chain derivative), so as to induce a double stranded cleavage at a site of interest of the NANOG gene comprising a DNA recognition and cleavage site for said meganuclease, simultaneously or consecutively,

(b) introducing into the cell of step (a), a targeting DNA, wherein said targeting DNA comprises (1) DNA sharing homologies to the region surrounding the cleavage site and (2) DNA which repairs the site of interest upon recombination between the targeting DNA and the chromosomal DNA, so as to generate a recombinant cell having repaired the site of interest by homologous recombination,

(c) isolating the recombinant cell of step (b), by any appropriate means.

The subject-matter of the present invention is also a method for making a NANOG gene knock-out or knock-in animal, comprising at least the step of:

(a) introducing into a pluripotent precursor cell or an embryo of an animal, a meganuclease as defined above, so as to induce a double stranded cleavage at a site of interest of the NANOG gene comprising a DNA recognition and cleavage site for said meganuclease, simultaneously or consecutively,

(b) introducing into the animal precursor cell or embryo of step (a) a targeting DNA, wherein said targeting DNA comprises (1) DNA sharing homologies to the region surrounding the cleavage site and (2) DNA which repairs the site of interest upon recombination between the targeting DNA and the chromosomal DNA, so as to generate a genetically modified animal precursor cell or embryo having repaired the site of interest by homologous recombination,

(c) developing the genetically modified animal precursor cell or embryo of step (b) into a chimeric animal, and

(d) deriving a transgenic animal from the chimeric animal of step (c).

Preferably, step (c) comprises the introduction of the genetically modified precursor cell generated in step (b) into blastocysts so as to generate chimeric animals.

The targeting DNA is introduced into the cell under conditions appropriate for introduction of the targeting DNA into the site of interest.

For making knock-out cells/animals, the DNA which repairs the site of interest comprises sequences that inactivate the NANOG gene.

For making knock-in cells/animals, the DNA which repairs the site of interest comprises the sequence of an exogenous gene of interest, and eventually a selection marker, such as the neomycin resistance gene.

In a preferred embodiment, said targeting DNA construct is inserted in a vector.

The subject-matter of the present invention is also a method for making a NANOG-deficient cell, comprising at least the step of:

(a) introducing into a cell, a meganuclease as defined above, so as to induce a double stranded cleavage at a site of interest of the NANOG gene comprising a DNA recognition and cleavage site of said meganuclease, and thereby generate genetically modified NANOG gene-deficient cell having repaired the double-strands break, by non-homologous end joining, and

(b) isolating the genetically modified NANOG gene-deficient cell of step (a), by any appropriate mean.

The subject-matter of the present invention is also a method for making a NANOG gene knock-out animal, comprising at least the step of:

(a) introducing into a pluripotent precursor cell or an embryo of an animal, a meganuclease, as defined above, so as to induce a double stranded cleavage at a site of interest of the NANOG gene comprising a DNA recognition and cleavage site of said meganuclease, and thereby generate genetically modified precursor cell or embryo having repaired the double-strands break by non-homologous end joining,

(b) developing the genetically modified animal precursor cell or embryo of step (a) into a chimeric animal, and

(c) deriving a transgenic animal from a chimeric animal of step (b).

Preferably, step (b) comprises the introduction of the genetically modified precursor cell obtained in step (a), into blastocysts, so as to generate chimeric animals.

The cells which are modified may be any cells of interest as long as they contain the specific target site. For making knock-in/transgenic mice, the cells are pluripotent precursor cells such as embryo-derived stem (ES) cells, which are well-known in the art. For making recombinant human cell lines, the cells may advantageously be PerC6 (Fallaux et al., Hum. Gene Ther. 9, 1909-1917, 1998) or HEK293 (ATCC # CRL-1573) cells.

The animal is preferably a mammal, more preferably a laboratory rodent (mice, rat, guinea-pig), or a rabbit, a cow, pig, horse or goat.

Said meganuclease can be provided directly to the cell or through an expression vector comprising the polynucleotide sequence encoding said meganuclease and suitable for its expression in the used cell.

For making recombinant cell lines expressing an heterologous protein of interest, the targeting DNA comprises a sequence encoding the product of interest (protein or RNA), and eventually a marker gene, flanked by sequences upstream and downstream the cleavage site, as defined above, so as to generate genetically modified cells having integrated the exogenous sequence of interest in the NANOG gene, by homologous recombination.

The sequence of interest may be any gene coding for a certain protein/peptide of interest, included but not limited to: reporter genes, receptors, signaling molecules, transcription factors, pharmaceutically active proteins and peptides, disease causing gene products and toxins. The sequence may also encode a RNA molecule of interest including for example an interfering RNA such as ShRNA, miRNA or siRNA, well-known in the art.

The expression of the exogenous sequence may be driven, either by the endogenous NANOG gene promoter or by a heterologous promoter, preferably an ubiquitous or tissue specific promoter, either constitutive or inducible, as defined above. In addition, the expression of the sequence of interest may be conditional; the expression may be induced by a site-specific recombinase such as Cre or FLP (Akagi K, Sandig V, Vooijs M, Van der Valk M, Giovannini M, Strauss M, Berns A (May 1997). “Nucleic Acids Res. 25 (9): 1766-73; Zhu X D, Sadowski P D (1995). J Biol Chem 270).

Thus, the sequence of interest is inserted in an appropriate cassette that may comprise an heterologous promoter operatively linked to said gene of interest and one or more functional sequences including but not limited to (selectable) marker genes, recombinase recognition sites, polyadenylation signals, splice acceptor sequences, introns, tag for protein detection and enhancers.

The subject matter of the present invention is also a kit for making NANOG gene knock-out or knock-in cells/animals comprising at least a meganuclease and/or one expression vector, as defined above. Preferably, the kit further comprises a targeting DNA comprising a sequence that inactivates the NANOG gene flanked by sequences sharing homologies with the region of the NANOG gene surrounding the DNA cleavage site of said meganuclease. In addition, for making knock-in cells/animals, the kit includes also a vector comprising a sequence of interest to be introduced in the genome of said cells/animals and eventually a selectable marker gene, as defined above.

The subject-matter of the present invention is also the use of at least one meganuclease and/or one expression vector, as defined above, for the preparation of a medicament for preventing, improving or curing a pathological condition caused by a mutation in the NANOG gene as defined above, in an individual in need thereof.

The use of the meganuclease may comprise at least the step of (a) inducing in somatic tissue(s) of the donor/individual a double stranded cleavage at a site of interest of the NANOG gene comprising at least one recognition and cleavage site of said meganuclease by contacting said cleavage site with said meganuclease, and (b) introducing into said somatic tissue(s) a targeting DNA, wherein said targeting DNA comprises (1) DNA sharing homologies to the region surrounding the cleavage site and (2) DNA which repairs the NANOG gene upon recombination between the targeting DNA and the chromosomal DNA, as defined above. The targeting DNA is introduced into the somatic tissues(s) under conditions appropriate for introduction of the targeting DNA into the site of interest.

According to the present invention, said double-stranded cleavage may be induced, ex vivo by introduction of said meganuclease into somatic cells from the diseased individual and then transplantation of the modified cells back into the diseased individual.

The subject-matter of the present invention is also a method for preventing, improving or curing a pathological condition caused by a mutation in the NANOG gene, in an individual in need thereof, said method comprising at least the step of administering to said individual a composition as defined above, by any means. The meganuclease can be used either as a polypeptide or as a polynucleotide construct encoding said polypeptide. It is introduced into mouse cells, by any convenient means well-known to those in the art, which are appropriate for the particular cell type, alone or in association with either at least an appropriate vehicle or carrier and/or with the targeting DNA.

According to an advantageous embodiment of the uses according to the invention, the meganuclease (polypeptide) is associated with:
liposomes, polyethyleneimine (PEI); in such a case said association is administered and therefore introduced into somatic target cells.
membrane translocating peptides (Bonetta, The Scientist, 2002, 16, 38; Ford et al., Gene Ther., 2001, 8, 1-4; Wadia and Dowdy, Curr. Opin. Biotechnol., 2002, 13, 52-56); in such a case, the sequence of the variant/single-chain meganuclease is fused with the sequence of a membrane translocating peptide (fusion protein).

According to another advantageous embodiment of the uses according to the invention, the meganuclease (polynucleotide encoding said meganuclease) and/or the targeting DNA is inserted in a vector. Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”). Optionally, it may be preferable to incorporate a nuclear localization signal into the recombinant protein to be sure that it is expressed within the nucleus.

Once in a cell, the meganuclease and if present, the vector comprising targeting DNA and/or nucleic acid encoding a meganuclease are imported or translocated by the cell from the cytoplasm to the site of action in the nucleus.

Since meganucleases recognize a specific DNA sequence, any meganuclease developed in the context of human gene therapy could be used in other contexts (other organisms, other loci, use in the context of a landing pad containing the site) unrelated with gene therapy of NANOG in human as long as the site is present.

For purposes of therapy, the meganucleases and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results in a decrease in the severity of one or more symptoms of the targeted disease and in a genome correction of the lesion or abnormality. Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”).

In one embodiment of the uses according to the present invention, the meganuclease is substantially non-immunogenic, i.e., engender little or no adverse immunological response. A variety of methods for ameliorating or eliminating deleterious immunological reactions of this sort can be used in accordance with the invention. In a preferred embodiment, the meganuclease is substantially free of N-formyl methionine. Another way to avoid unwanted immunological reactions is to conjugate meganucleases to polyethylene glycol (“PEG”) or polypropylene glycol (“PPG”) (preferably of 500 to 20,000 daltons average molecular weight (MW)). Conjugation with PEG or PPG, as described by Davis et al. (U.S. Pat. No. 4,179,337) for example, can provide non-immunogenic, physiologically active, water soluble endonuclease conjugates with anti-viral activity. Similar methods also using a polyethylene—polypropylene glycol copolymer are described in Saifer et al. (U.S. Pat. No. 5,006,333).

The invention also concerns a prokaryotic or eukaryotic host cell which is modified by a polynucleotide or a vector as defined above, preferably an expression vector.

The invention also concerns a non-human transgenic animal or a transgenic plant, characterized in that all or a part of their cells are modified by a polynucleotide or a vector as defined above.

As used herein, a cell refers to a prokaryotic cell, such as a bacterial cell, or an eukaryotic cell, such as an animal, plant or yeast cell.

The subject-matter of the present invention is also the use of at least one meganuclease variant, as defined above, as a scaffold for making other meganucleases. For example, further rounds of mutagenesis and selection/screening can be performed on said variants, for the purpose of making novel meganucleases.

The different uses of the meganuclease and the methods of using said meganuclease according to the present invention include the use of the I-CreI variant, the single-chain chimeric meganuclease derived from said variant, the polynucleotide(s), vector, cell, transgenic plant or non-human transgenic mammal encoding said variant or single-chain chimeric meganuclease, as defined above.

Single-chain chimeric meganucleases able to cleave a DNA target from the gene of interest are derived from the variants according to the invention by methods well-known in the art (Epinat et al., Nucleic Acids Res., 2003, 31, 2952-62; Chevalier et al., Mol. Cell., 2002, 10, 895-905; Steuer et al., Chembiochem., 2004, 5, 206-13; International PCT Applications WO 03/078619, WO 2004/031346 and WO 2009/095793). Any of such methods, may be applied for constructing single-chain chimeric meganucleases derived from the variants as defined in the present invention. In particular, the invention encompasses also the I-CreI variants defined in the tables II and III.

The polynucleotide sequence(s) encoding the variant as defined in the present invention may be prepared by any method known by the man skilled in the art. For example, they are amplified from a cDNA template, by polymerase chain reaction with specific primers. Preferably the codons of said cDNA are chosen to favour the expression of said protein in the desired expression system.

The recombinant vector comprising said polynucleotides may be obtained and introduced in a host cell by the well-known recombinant DNA and genetic engineering techniques.

The I-CreI variant or single-chain derivative as defined in the present invention are produced by expressing the polypeptide(s) as defined above; preferably said polypeptide(s) are expressed or co-expressed (in the case of the variant only) in a host cell or a transgenic animal/plant modified by one expression vector or two expression vectors (in the case of the variant only), under conditions suitable for the expression or co-expression of the polypeptide(s), and the variant or single-chain derivative is recovered from the host cell culture or from the transgenic animal/plant.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

### DEFINITIONS

Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Altered/enhanced/increased cleavage activity, refers to an increase in the detected level of meganuclease cleavage activity, see below, against a target DNA sequence by a second meganuclease in comparison to the activity of a first meganuclease against the target DNA sequence. Normally the second meganuclease is a variant of the first and comprise one or more substituted amino acid residues in comparison to the first meganuclease.
iPS or iPSC refer to induced Pluripotent Stem Cells.
by “clean iPS” cells is intended iPS cells in which transgenes that have been first inserted in their genomes for their reprogrammation toward said iPS, have been secondarily removed without any scar in their genome for obtaining such clean iPS, avoiding problems in further re-differentiation steps and therapeutic uses due to the permanent expression of these transgenes in classical approach.
by “safe iPS” is intended iPS cells that have lost self-renewable property for example by knocking-out at least a gene conferring or implicated in said self-renewable cellular property.
by “secure iPS” cells is intended iPS cells in which, at a defined step of differentiation process, the progression of iPS cells toward more differentiated cell types is made irreversible.
by “clean and/or safe and/or secure” iPS is intended iPS cells comprising one or more of the previously-described properties.
by reprogrammation process is intended the process of dedifferentiation of a somatic cell toward iPS cells.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
by “endonuclease” is intended any wild-type or variant enzyme capable of catalyzing the hydrolysis (cleavage) of bonds between nucleic acids within of a DNA or RNA molecule, preferably a DNA molecule. Endonucleases do not cleave the DNA or RNA molecule irrespective of its sequence, but recognize and cleave the DNA or RNA molecule at specific polynucleotide sequences, further referred to as “target sequences” or “target sites” and significantly increased HR by specific meganuclease-induced DNA double-strand break (DSB) at a defined locus (Rouet et al, 1994; Choulika et al, 1995). Endonucleases can for example be a homing endonuclease (Paques et al. Curr Gen Ther. 2007 7:49-66), a chimeric Zinc-Finger nuclease (ZFN) resulting from the fusion of engineered zinc-finger domains with the catalytic domain of a restriction enzyme such as FokI (Porteus et al. Nat. Biotechnol. 2005 23:967-973) or a chemical endonuclease (Arimondo et al. Mol Cell Biol. 2006 26:324-333; Simon et al. NAR 2008 36:3531-3538; Eisenschmidt et al. NAR 2005 33:7039-7047; Cannata et al. PNAS 2008 105:9576-9581). In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences (Kalish and Glazer Ann NY Acad Sci 2005 1058: 151-61). Such chemical endonucleases are comprised in the term “endonuclease” according to the present invention. In the scope of the present invention is also intended any fusion between molecules able to bind DNA specific sequences and agent/reagent/chemical able to cleave DNA or interfere with cellular proteins implicated in the DSB repair (Majumdar et al. J. Biol. Chem. 2008 283, 17:11244-11252; Liu et al. NAR 2009 37:6378-6388); as a non limiting example such a fusion can be constituted by a specific DNA-sequence binding domain linked to a chemical inhibitor known to inhibate religation activity of a topoisomerase after DSB cleavage. Endonuclease can be a homing endonuclease, also known under the name of meganuclease. By “meganuclease”, is intended an endonuclease having a double-stranded DNA target sequence of 12 to 45 bp. Such homing endonucleases are well-known to the art (see e.g. Stoddard, Quarterly Reviews of Biophysics, 2006, 38:49-95). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease according to the invention may for example correspond to a LAGLIDADG endonuclease, to a HNH endonuclease, or to a GIY-YIG endonuclease. Said meganuclease is either a dimeric enzyme, wherein each domain is on a monomer or a monomeric enzyme comprising the two domains on a single polypeptide.

Endonucleases according to the invention can also be derived from TALENs, a new class of chimeric nucleases using a FokI catalytic domain and a DNA binding domain derived from Transcription Activator Like Effector (TALE), a family of proteins used in the infection process by plant pathogens of the Xanthomonas genus (Boch, Scholze et al. 2009; Moscou and Bogdanove 2009; Christian, Cermak et al. 2010; Li, Huang et al. 2011) (Boch, Scholze et al. 2009; Moscou and Bogdanove 2009; Christian, Cermak et al. 2010; Li, Huang et al. 2010). The functional layout of a FokI-based TALE-nuclease (TALEN) is essentially that of a ZFN, with the Zinc-finger DNA binding domain being replaced by the TALE domain. As such, DNA cleavage by a TALEN requires two DNA recognition regions flanking an unspecific central region. Endonucleases encompassed in the present invention can also be derived from TALENs. An endonuclease according to the present invention can be derived from a TALE-nuclease (TALEN), i.e. a fusion between a DNA-binding domain derived from a Transcription Activator Like Effector (TALE) and one or two catalytic domains.
by “meganuclease domain” is intended the region which interacts with one half of the DNA target of a meganuclease and is able to associate with the other domain of the same meganuclease which interacts with the other half of the DNA target to form a functional meganuclease able to cleave said DNA target.
by “meganuclease variant” or “variant” it is intended a meganuclease obtained by replacement of at least one residue in the amino acid sequence of the parent meganuclease with a different amino acid.
by “peptide linker” it is intended to mean a peptide sequence of at least 10 and preferably at least 17 amino acids which links the C-terminal amino acid residue of the first monomer to the N-terminal residue of the second monomer and which allows the two variant monomers to adopt the correct conformation for activity and which does not alter the specificity of either of the monomers for their targets.
by “subdomain” it is intended the region of a LAGLIDADG homing endonuclease core domain which interacts with a distinct part of a homing endonuclease DNA target half-site.
by “targeting DNA construct/minimal repair matrix/repair matrix” it is intended to mean a DNA construct comprising a first and second portions which are homologous to regions 5′ and 3′ of the DNA target in situ. The DNA construct also comprises a third portion positioned between the first and second portion which comprise some homology with the corresponding DNA sequence in situ or alternatively comprise no homology with the regions 5′ and 3′ of the DNA target in situ. Following cleavage of the DNA target, a homologous recombination event is stimulated between the genome containing the NANOG gene and the repair matrix, wherein the genomic sequence containing the DNA target is replaced by the third portion of the repair matrix and a variable part of the first and second portions of the repair matrix.
by “functional variant” is intended a variant which is able to cleave a DNA target sequence, preferably said target is a new target which is not cleaved by the parent meganuclease. For example, such variants have amino acid variation at positions contacting the DNA target sequence or interacting directly or indirectly with said DNA target.
by “selection or selecting” it is intended to mean the isolation of one or more meganuclease variants based upon an observed specified phenotype, for instance altered cleavage activity. This selection can be of the variant in a peptide form upon which the observation is made or alternatively the selection can be of a nucleotide coding for selected meganuclease variant.
by “screening” it is intended to mean the sequential or simultaneous selection of one or more meganuclease variant (s) which exhibits a specified phenotype such as altered cleavage activity.
by “derived from” it is intended to mean a meganuclease variant which is created from a parent meganuclease and hence the peptide sequence of the meganuclease variant is related to (primary sequence level) but derived from (mutations) the sequence peptide sequence of the parent meganuclease.
by “I-CreI” is intended the wild-type I-CreI having the sequence of pdb accession code 1g9y, corresponding to the sequence SEQ ID NO: 1 in the sequence listing.
by “I-CreI variant with novel specificity” is intended a variant having a pattern of cleaved targets different from that of the parent meganuclease. The terms “novel specificity”, “modified specificity”, “novel cleavage specificity”, “novel substrate specificity” which are equivalent and used indifferently, refer to the specificity of the variant towards the nucleotides of the DNA target sequence. In the present patent application all the I-CreI variants described comprise an additional Alanine after the first Methionine of the wild type I-CreI sequence (SEQ ID NO: 65). These variants also comprise two additional Alanine residues and an Aspartic Acid residue after the final Proline of the wild type I-CreI sequence. These additional residues do not affect the properties of the enzyme and to avoid confusion these additional residues do not affect the numeration of the residues in I-CreI or a variant referred in the present patent application, as these references exclusively refer to residues of the wild type I-CreI enzyme (SEQ ID NO: 1) as present in the variant, so for instance residue 2 of I-CreI is in fact residue 3 of a variant which comprises an additional Alanine after the first Methionine.
by “I-CreI site” is intended a 22 to 24 bp double-stranded DNA sequence which is cleaved by I-CreI. I-CreI sites include the wild-type non-palindromic I-CreI homing site and the derived palindromic sequences such as the sequence 5′-t₋₁₂c₋₁₁a₋₁₀a₋₉a₋₈a₋₇c₋₆g₋₅t₋₄c₋₃g₋₂t₋₁a₊₁c₊₂g₊₃a₊₄c₊₅g₊₆t₊₇t₊₈t₊₉t₊₁₀g₊₁₁a₊₁₂ (SEQ ID NO: 2), also called C1221 (FIGS. 3 and 5).
by “domain” or “core domain” is intended the “LAGLIDADG homing endonuclease core domain” which is the characteristic α₁β₁β₂α₂β₃β₄α₃ fold of the homing endonucleases of the LAGLIDADG family, corresponding to a sequence of about one hundred amino acid residues. Said domain comprises four beta-strands (β₁β₂β₃β₄) folded in an anti-parallel beta-sheet which interacts with one half of the DNA target. This domain is able to associate with another LAGLIDADG homing endonuclease core domain which interacts with the other half of the DNA target to form a functional endonuclease able to cleave said DNA target. For example, in the case of the dimeric homing endonuclease I-CreI (163 amino acids), the LAGLIDADG homing endonuclease core domain corresponds to the residues 6 to 94.
by “subdomain” is intended the region of a LAGLIDADG homing endonuclease core domain which interacts with a distinct part of a homing endonuclease DNA target half-site.
by “chimeric DNA target” or “hybrid DNA target” it is intended the fusion of a different half of two parent meganuclease target sequences. In addition at least one half of said target may comprise the combination of nucleotides which are bound by at least two separate subdomains (combined DNA target).
by “beta-hairpin” is intended two consecutive beta-strands of the antiparallel beta-sheet of a LAGLIDADG homing endonuclease core domain (β₁β₂ or, β₃β₄) which are connected by a loop or a turn,

by “single-chain meganuclease”, “single-chain chimeric meganuclease”, “single-chain meganuclease derivative”, “single-chain chimeric meganuclease derivative” or “single-chain derivative” is intended a meganuclease comprising two LAGLIDADG homing endonuclease domains or core domains linked by a peptidic spacer. The single-chain meganuclease is able to cleave a chimeric DNA target sequence comprising one different half of each parent meganuclease target sequence.
by “DNA target”, “DNA target sequence”, “target sequence”, “target-site”, “target”, “site”, “site of interest”, “recognition site”, “recognition sequence”, “homing recognition site”, “homing site”, “cleavage site” is intended a 20 to 24 bp double-stranded palindromic, partially palindromic (pseudo-palindromic) or non-palindromic polynucleotide sequence that is recognized and cleaved by a LAGLIDADG homing endonuclease such as I-CreI, or a variant, or a single-chain chimeric meganuclease derived from I-CreI. These terms refer to a distinct DNA location, preferably a genomic location, at which a double stranded break (cleavage) is to be induced by the meganuclease. The DNA target is defined by the 5′ to 3′ sequence of one strand of the double-stranded polynucleotide, as indicate above for C1221. Cleavage of the DNA target occurs at the nucleotides at positions +2 and −2, respectively for the sense and the antisense strand. Unless otherwise indicated, the position at which cleavage of the DNA target by an I-Cre I meganuclease variant occurs, corresponds to the cleavage site on the sense strand of the DNA target.
by “DNA target half-site”, “half cleavage site” or half-site” is intended the portion of the DNA target which is bound by each LAGLIDADG homing endonuclease core domain.
by “chimeric DNA target” or “hybrid DNA target” is intended the fusion of different halves of two parent meganuclease target sequences. In addition at least one half of said target may comprise the combination of nucleotides which are bound by at least two separate subdomains (combined DNA target).
by “gene” is intended the basic unit of heredity, consisting of a segment of DNA arranged in a linear manner along a chromosome, which encodes for a specific protein or segment of protein. A gene typically includes a promoter, a 5′ untranslated region, one or more coding sequences (exons), optionally introns, a 3′ untranslated region. The gene may further comprise a terminator, enhancers and/or silencers. by “gene” is also intended one or several part of this gene, as listed above.
by “NANOG gene”, is preferably intended a NANOG gene of a vertebrate or part of it, more preferably the NANOG gene or part of it of a mammal such as human. NANOG gene sequences are available in sequence databases, such as the NCBI/GenBank database. This gene has been described in databanks as NC000012 entry (NCBI).
by “DNA target sequence from the NANOG gene”, “genomic DNA target sequence”, “genomic DNA cleavage site”, “genomic DNA target” or “genomic target” is intended a 22 to 24 bp sequence of the NANOG gene as defined above, which is recognized and cleaved by a meganuclease variant or a single-chain chimeric meganuclease derivative.
by “parent meganuclease” it is intended to mean a wild type meganuclease or a variant of such a wild type meganuclease with identical properties or alternatively a meganuclease with some altered characteristic in comparison to a wild type version of the same meganuclease. In the present invention the parent meganuclease can refer to the initial meganuclease from which a series of variants are derived from.
by “vector” is intended a nucleic acid molecule capable of transporting another nucleic acid to which it has been linked.
by “homologous” is intended a sequence with enough identity to another one to lead to homologous recombination between sequences, more particularly having at least 95% identity, preferably 97% identity and more preferably 99% or 99.5%.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting.
by “mutation” is intended the substitution, deletion, insertion of one, two, three, four, five, six, ten or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.
“gene of interest” or “GOI” refers to any nucleotide sequence encoding a known or putative gene product.

—As used herein, the term “locus” is the specific physical location of a DNA sequence (e.g. of a gene) on a chromosome. The term “locus” usually refers to the specific physical location of an endonuclease's target sequence on a chromosome. Such a locus, which comprises a target sequence that is recognized and cleaved by an endonuclease according to the invention, is referred to as “locus according to the invention”.
by “safe harbor” locus of the genome of a cell, tissue or individual, is intended a gene locus wherein a transgene could be safely inserted, the disruption or deletion of said locus consecutively to the insertion not modifying expression of genes located outside of said locus, NANOG gene being a good safe harbor locus because this gene is silent in normal cells and only express in iPS cells or cancer cells.
As used herein, the term “transgene” refers to a sequence encoding a polypeptide. Preferably, the polypeptide encoded by the transgene is either not expressed, or expressed but not biologically active, in the cell, tissue or individual in which the transgene is inserted. Most preferably, the transgene encodes a therapeutic polypeptide useful for the treatment of an individual.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

The following non-limiting examples illustrate some aspects of the invention.

### EXAMPLES

### Example 1

### Engineering Meganucleases Targeting the NANOG2 Site

### Protein Design

I-CreI variants targeting the NANOG2 site were created using a combinatorial approach, to entirely redesign the DNA binding domain of the I-CreI protein and thereby engineer novel meganucleases with fully engineered specificity for the desired NANOG gene target. Some of the DNA targets identified by the inventors which validate the overall concept of the invention are shown in Table I above. Derivatives of these DNA targets are given in FIGS. 3 & 5. The combinatorial approach, as illustrated in FIG. 2 and described in International PCT applications WO 2006/097784 and WO 2006/097853, and also in Arnould et al. (J. Mol. Biol., 2006, 355, 443-458) and Smith et al. (Nucleic Acids Res., 2006) was used to redesign the DNA binding domain of the I-CreI protein and thereby engineer novel meganucleases with fully engineered specificity.

a) Construction of Variants Targeting the NANOG2 Site

NANOG2 site is an example of a target for which meganuclease variants have been generated. The NANOG2 target sequence or NANOG 2.1 (CC-AAC-AT-CCT-GAAC-CTC-AG-CTA-CA, SEQ ID NO: 8) is located in exon 2 of NANOG gene at positions 3786 to 3809 of NC000012 entry (NCBI).

The NANOG2.1 sequence is partially a combination of the 10AAC_P (SEQ ID NO: 4), 5CCT_P (SEQ ID NO: 5), 10TAG_P (SEQ ID NO: 6) and 5GAG_P (SEQ ID NO: 7) target sequences which are shown on FIG. 3. These sequences are cleaved by meganucleases obtained as described in International PCT applications WO 2006/097784 and WO 2006/097853, Arnould et al. (J. Mol. Biol., 2006, 355, 443-458) and Smith et al. (Nucleic Acids Res., 2006).

Two palindromic targets, NANOG2.3 (SEQ ID NO: 10) and NANOG2.4 (SEQ ID NO: 11), and two pseudo palindromic targets, NANOG2.5 (SEQ ID NO: 12) and NANOG2.6 (SEQ ID NO: 13), were derived from NANOG2.1 (SEQ ID NO: 8) and NANOG2.2 (SEQ ID NO: 9) (FIG. 3). Since NANOG2.3 and NANOG2.4 are palindromic, they are cleaved by homodimeric proteins. Therefore, homodimeric I-CreI variants cleaving either the NANOG2.3 palindromic target sequence of SEQ ID NO: 10 or the NANOG2.4 palindromic target sequence of SEQ ID NO: 11 were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol. Biol. 2007 371:49-65).

Single chain obligate heterodimer constructs were generated for the I-CreI variants able to cleave the NANOG2 target sequences when forming heterodimers. These single chain constructs were engineered using the linker RM2: (AAGGSDKYNQALSKYNQALSKYNQALSGGGGS) (SEQ ID NO: 24).

During this design step, mutations K7E, K96E were introduced into the mutant cleaving NANOG2.3 (monomer 1) and mutations E8K, G19S, E61R into the mutant cleaving NANOG2.4 (monomer 2) to create the single chain molecules: monomer1 (K7E, K96E)-RM2-monomer2 (E8K, G19S, E61R) that is called SCOH-NANOG2 (Table II). Four additional amino-acid substitutions were found in previous studies that enhance the activity of I-CreI derivatives: these mutations correspond to the replacement of Phenylalanine 54 with Leucine (F54L), Glutamic acid 80 with Lysine (E80K), Valine 105 with Alanine (V105A) and Isoleucine 132 with Valine (1132V). Some combinations were introduced into the coding sequence of N-terminal and C-terminal protein fragment, and some of the resulting proteins were assayed for their ability to induce cleavage of the NANOG2 target.

TABLE II
Example of SCOH-NANOG2 useful for NANOG2 targeting

| Single Chain- | | | | |
|---|---|---|---|---|
| encoding | | | | |
| Plasmid | | | | |
| (SCOH- | Nterminal mutations in | Cterminal mutations in | Cleavage in | SC |
| NANOG2) | Single Chains (SC) | Single Chains (SC) | CHO | SEQ ID NO: |
| pCLS4412 | 6T7E28R33R38Y40Q44 | 8K19S32N33C40R61R68 | + | 25 |
| (SEQ ID: 41) | K68A70G75N96E132V | Y70S75Y77Y | | |
| pCLS4413 | 6T7E28R33R38Y40Q44 | 8K19S32N33C40R61R | + | 26 |
| (SEQ ID: 42) | K68A70G75N96E132V | 68Y70S75Y77Y132V | | |
| pCLS4414 | 7E28R33R38Y40Q44K6 | 8K19S30H33C40R44Y61 | + | 27 |
| (SEQ ID: 43) | 8A70G75N96E | R 68Y70S75N77T | | |
| pCLS4415 | 7E28R33R38Y40Q44K6 | 8K19S30H33C40R44Y61 | + | 28 |
| (SEQ ID: 44) | 8A70G75N96E132V | R 68Y70S75N77T132V | | |
| pCLS4416 | 7E28R33R38Y40Q44K6 | 8K19S30H33C40R44Y61 | + | 29 |
| (SEQ ID: 45) | 8A70G75N80K96E132V | R 68Y70S75N77T132V | | |
| pCLS4417 | 7E28R33R38Y40Q44K5 | 8K19S30H33C40R44Y61 | + | 30 |
| (SEQ ID: 46) | 4I64A68A70G75N96E1 | R 68Y70S75N77T | | |
| | 47A | | | |
| pCLS4418 | 7E28R33R38Y40Q44K5 | 8K19S30H33C40R44Y61 | + | 31 |
| (SEQ ID: 47) | 4I64A68A70G75N96E1 | R 68Y70S75N77T132V | | |
| | 32V147A | | | |
| pCLS4419 | 7E28R33R38Y40Q44K5 | 8K19S30H33C40R44Y61 | + | 32 |
| (SEQ ID: 48) | 4I64A68A70G75N80K9 | R 68Y70S75N77T132V | | |
| | 6E132V147A | | | |

b) Validation of Some SCOH-NANOG2 Variants in a Mammalian Cells Extrachromosomal Assay.

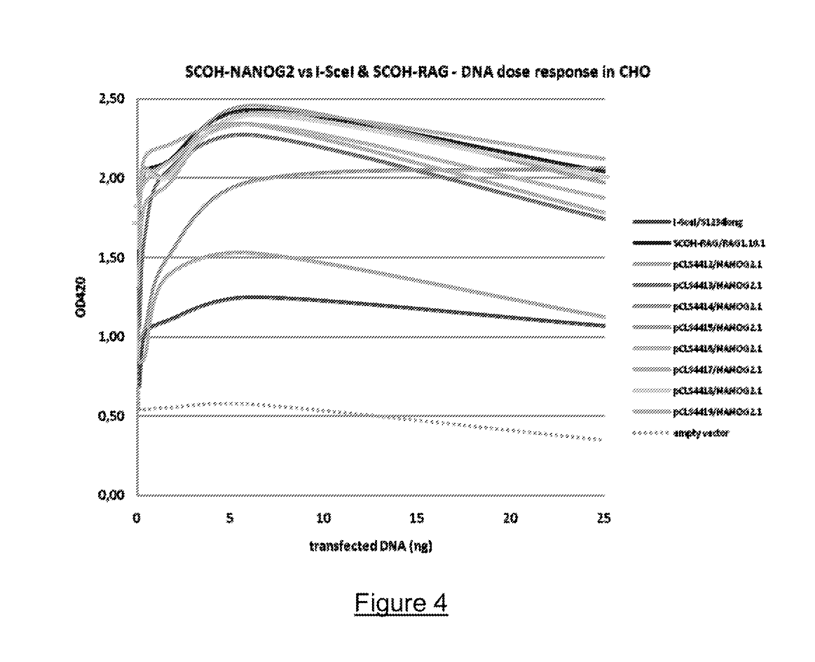

The activity of the single chain molecules against the NANOG2 target was monitored using the described CHO assay along with our internal control SCOH-RAG and I-Sce I meganucleases. All comparisons were done from 0.02 to 25 ng transfected variant DNA (FIG. 4). All the single molecules displayed NANOG2 target cleavage activity in CHO assay as listed in Table II. Variants shared specific behavior upon assayed dose depending on the mutation profile they bear (FIG. 4). For example, all but pCLS4412 and pCLS4414 have a similar profile and activity range than our standard control SCOH-RAG (pCLS2222) at low doses, reaches and maxima and decrease with increasing DNA doses. pCLS4412 has a similar profile than our standard and display an activity in a similar range than I-SceI. pCLS4414 displays an intermediate activity from I-Sce I and our SCOH-RAG standard at low doses but reaches a stable plateau up to 25 ng of transfected DNA. All of the variants described are strongly active and can be used for targeting genes into the NANOG2 locus.

### Example 2

### Engineering Meganucleases Targeting the NANOG4 Site

a) Construction of Variants Targeting the NANOG4 Site

NANOG4 site is an example of a target for which meganuclease variants have been generated. The NANOG4 target sequence or NANOG 4.1 (AC-TGA-AC-GCT-GTAA-AAT-AG-CTT-AA, SEQ ID NO: 18) is located in intron 1 of NANOG gene at positions 1222-1245 of NC000012 entry (NCBI).

The NANOG4 sequence is partially a combination of the 10TGA_P (SEQ ID NO: 14), 5GCT_P (SEQ ID NO: 15), 10AAG_P (SEQ ID NO: 16) and 5ATT_P (SEQ ID NO: 17) target sequences which are shown on FIG. 5. These sequences are cleaved by mega-nucleases obtained as described in International PCT applications WO 2006/097784 and WO 2006/097853, Arnould et al. (J. Mol. Biol., 2006, 355, 443-458) and Smith et al. (Nucleic Acids Res., 2006).

Two palindromic targets, NANOG4.3 (SEQ ID NO: 20) and NANOG4.4 (SEQ ID NO: 21) and two pseudo palindromic targets, NANOG4.5 (SEQ ID NO: 22) and NANOG4.6 (SEQ ID NO: 23), were derived from NANOG4.1 ((SEQ ID NO: 18) and NANOG4.2 (SEQ ID NO: 19) (FIG. 5). Since NANOG4.3 and NANOG4.4 are palindromic, they are cleaved by homodimeric proteins. Therefore, homodimeric I-CreI variants cleaving either the NANOG4.3 palindromic target sequence of SEQ ID NO or the NANOG4.4 palindromic target sequence of SEQ ID NO were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol. Biol. 2007 371:49-65).

Single chain obligate heterodimer constructs were generated for the I-CreI variants able to cleave the NANOG4 target sequences when forming heterodimers. These single chain constructs were engineered using the linker RM2 (AAGGSDKYNQALSKYNQALSKYNQALSGGGGS) (SEQ ID NO:24).

During this design step, mutations K7E, K96E were introduced into the mutant cleaving NANOG4.3 (monomer 1) and mutations E8K, G19S, E61R into the mutant cleaving NANOG4.4 (monomer 2) to create the single chain molecules: monomer1 (K7E K96E)-RM2-monomer2 (E8K G19S E61R) that is called SCOH-NANOG4 (Table III).

Four additional amino-acid substitutions were found in previous studies to enhance the activity of I-CreI derivatives: these mutations correspond to the replacement of Phenylalanine 54 with Leucine (F54L), Glutamic acid 80 with Lysine (E80K), Valine 105 with Alanine (V105A) and Isoleucine 132 with Valine (I132V). Some combinations were introduced into the coding sequence of N-terminal and C-terminal protein fragment, and some of the resulting proteins were assayed for their ability to induce cleavage of the NANOG4 target.

TABLE III
example of SCOH-NANOG4 useful for NANOG4 targeting

| Single Chain- | | | | |
|---|---|---|---|---|
| encoding | | | | |
| plasmid | | | | |
| (SCOH- | Nterminal mutations in Single | Cterminal mutations in | Cleavage in | SC |
| NANOG4) | Chains (SC) | Single Chains (SC) | CHO | SEQ ID NO: |
| pCLS4420 | 7E33T38R40Q43L44Y54C68 | 8K19S30G40Y61R70S | + | 33 |
| (SEQ ID: 49) | E70S75R77V96E | 75N81V87L | | |
| pCLS4421 | 7E33T38R40Q43L44Y54C68 | 8K19S30G40Y61R70S | + | 34 |
| (SEQ ID: 50) | E70S75R77V96E132V | 75N81V87L132V | | |
| pCLS4422 | 7E33T38R40Q43L44Y54C68 | 8K19S30G40Y61R70S | + | 35 |
| (SEQ ID: 51) | E70S75R77V80K96E132V | 75N81V87L132V | | |
| pCLS4697 | 7E33T38R40Q43L44Y54C68 | 8K19S11M40Y61R70S | + | 36 |
| (SEQ ID: 52) | E70S75R77V96E | 75N143N | | |
| pCLS4698 | 7E33T38R40Q43L44Y54C68 | 8K19S11M40Y61R70S | + | 37 |
| (SEQ ID: 53) | E70S75R77V96E132V | 75N132V143N | | |
| pCLS4699 | 7E33T38R40Q43L44Y54C68 | 8K19S11M40Y61R70S | + | 38 |
| (SEQ ID: 54) | E70S75R77V80K96E132V | 75N132V143N | | |
| pCLS4701 | 7E33T38R40Q43L44Y54C68 | 8K19S30G40Y54V61R | + | 39 |
| (SEQ ID: 55) | E70S75R77V96E132V | 70S75N81V132V | | |
| pCLS4702 | 7E33T38R40Q43L44Y54C68 | 8K19S30G40Y54V61R | + | 40 |
| (SEQ ID: 56) | E70S75R77V80K96E132V | 70S75N81V132V | | |

a) Validation of Some SCOH-NANOG4 Variants in a Mammalian Cells Extrachromosomal Assay.

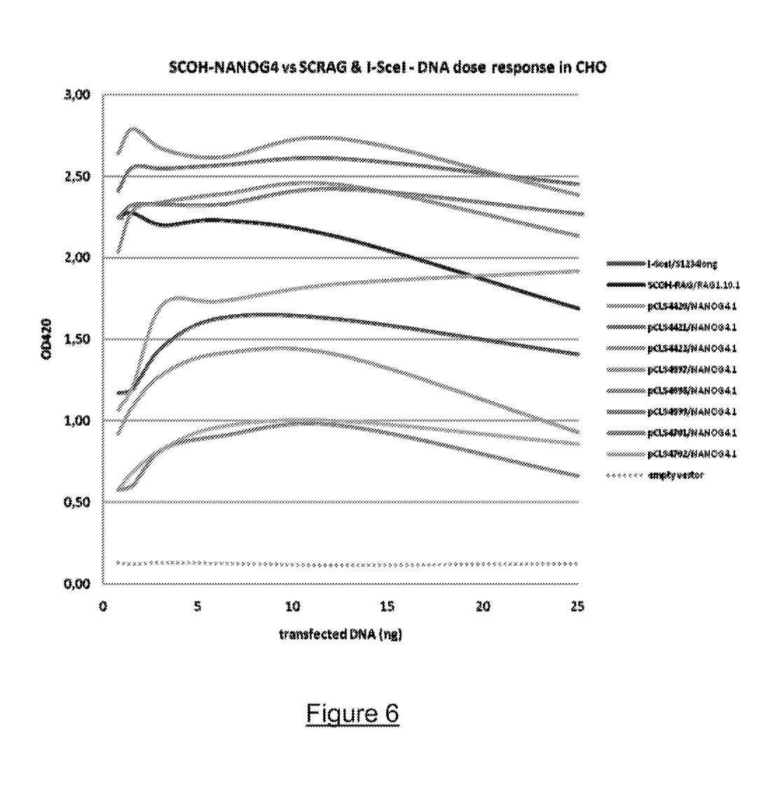

The activity of the single chain molecules against the NANOG4 target was monitored using the described CHO assay along with our internal control SCOH-RAG and I-Sce I meganucleases. All comparisons were done from 0.8 to 25 ng transfected variant DNA (FIG. 6). All the single molecules displayed NANOG4 target cleavage activity in CHO assay as listed in Table III. Variants shared specific behavior upon assayed dose depending on the mutation profile they bear (FIG. 6). For example, pCLS4421, pCLS4422, pCLS4698 and pCLS4699 have a higher activity range than our standard control SCOH-RAG (pCLS2222). They reach an activity plateau at low doses, stable with increasing DNA doses. pCLS4697, pCLS4701 and pCLS4702 have a similar profile than our standards and display an activity in a similar range than I-SceI. pCLS4420 displays an intermediate activity from I-Sce I and our SCOH-RAG standard at low doses but reaches a maxima at higher doses than 25 ng of transfected DNA. All of the variants described are strongly active and can be used for targeting genes into the NANOG4 locus.

### Example 3

### Cloning and Extrachromosomal Assay in Mammalian Cells

a) Cloning of NANOG2 and NANOG4 Targets in a Vector for CHO Screen

The targets were cloned as follows using oligonucleotide corresponding to the target sequence flanked by gateway cloning sequence; the following oligonucleotides were ordered from PROLIGO. These oligonucleotides have the following sequences:

NANOG2:
(SEQ ID NO: 57)
5′-TGGCATACAAGTTTCCAACATCCTGAACCTCAGCTACACAATCGTC
TGTCA-3′,
NANOG4:
(SEQ ID NO: 58)
5′-TGGCATACAAGTTTACTGAACGCTGTAAAATAGCTTAACAATCGTC
TGTCA-3′,

Double-stranded target DNA, generated by PCR amplification of the single stranded oligonucleotide, was cloned using the Gateway protocol (INVITROGEN) into CHO reporter vector (pCLS1058). Target was cloned and verified by sequencing (MILLEGEN).

b) Cloning of the Single Chain Molecules

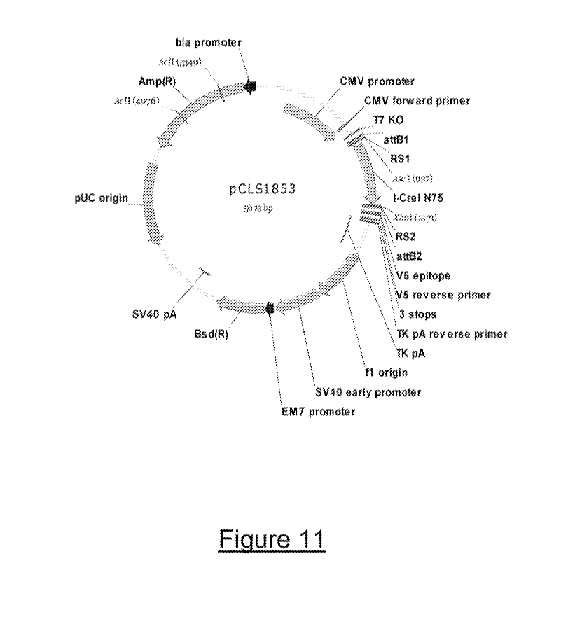

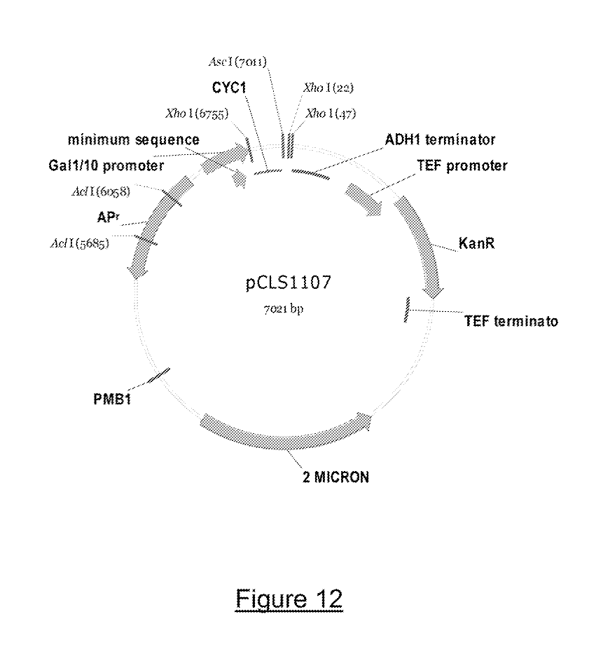

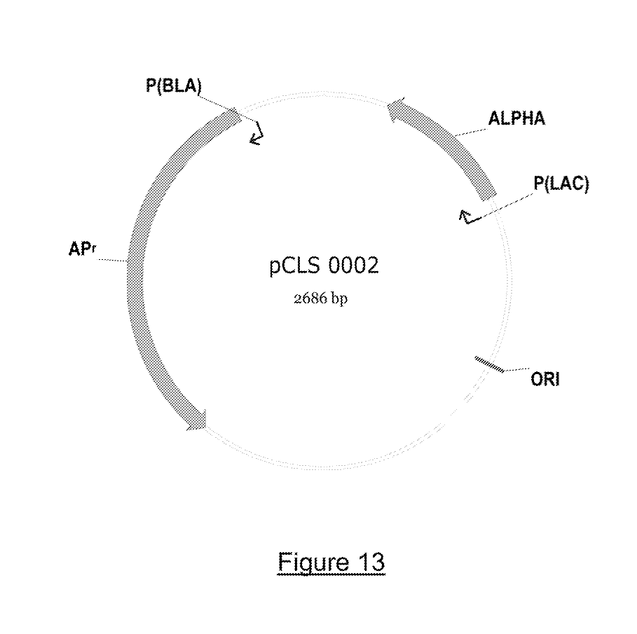

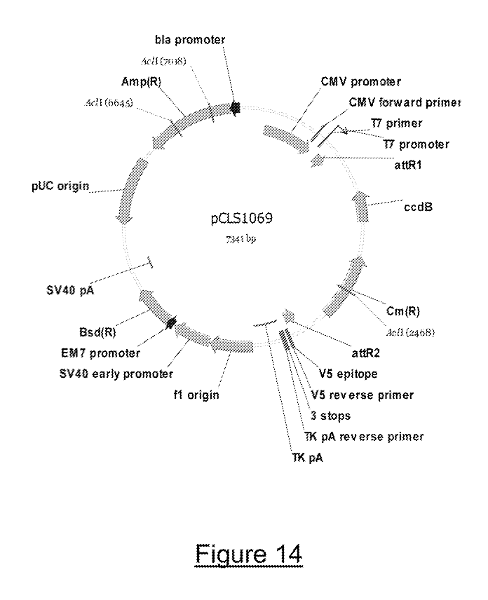

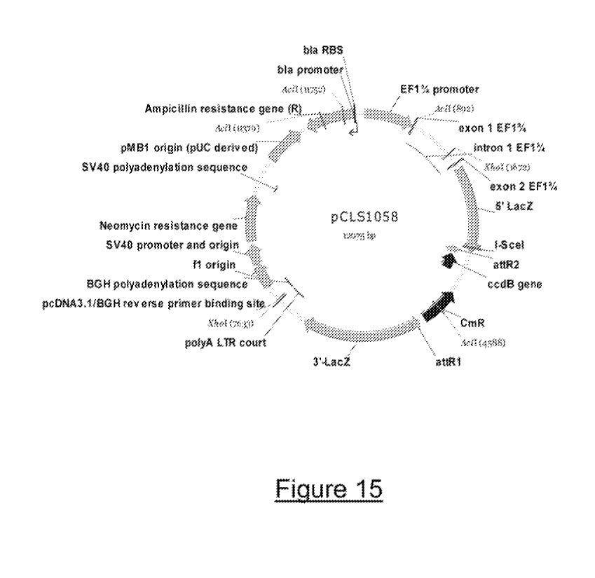

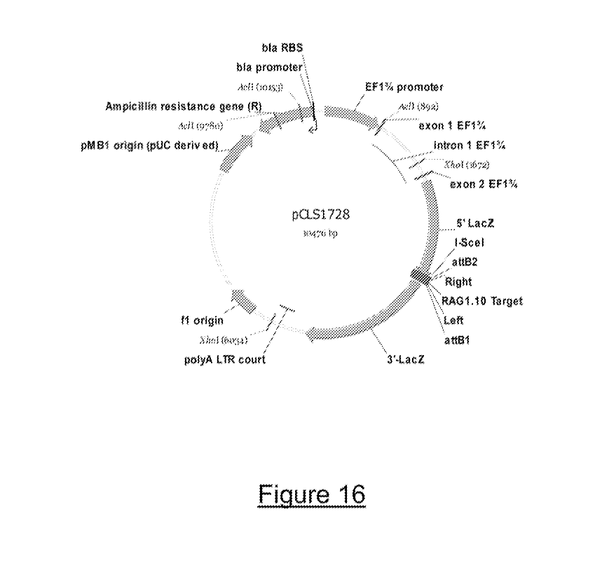

A series of synthetic gene assembly was ordered to Gene Cust. Synthetic genes coding for the different single chain variants targeting NANOG gene were cloned in pCLS1853 (FIG. 11) using AscI and XhoI restriction sites.

c) Extrachromosomal Assay in Mammalian Cells

CHO K1 cells were transfected as described in example 1.2. 72 hours after transfection, culture medium was removed and 150 μl of lysis/revelation buffer for β-galactosidase liquid assay was added. After incubation at 37° C., OD was measured at 420 nm. The entire process is performed on an automated Velocity11 BioCel platform. Per assay, 150 ng of target vector was cotransfected with an increasing quantity of variant DNA from 0.02 or 0.8 to 25 ng. The total amount of transfected DNA was completed to 175 ng (target DNA, variant DNA, carrier DNA) using an empty vector (pCLS0002).

Numerous modifications and variations on the present invention are possible in light of the above teachings. It is, therefore, to be understood that within the scope of the accompanying claims, the invention may be practiced otherwise than as specifically described herein.

### Example 4

### Detection of Induced Mutagenesis at the Endogenous Site

Genomic DNA double strand break (DSB) can be repaired by homologous recombination (HR) or Non-homologous end joining (NHEJ). If the homologous recombination can restore the genomic integrity, NHEJ is though to be an error-prone mechanism which results in small insertion or deletion (InDel) at the DSB. Therefore, the detection of the mutagenesis induced by a meganuclease at its cognate endogenous locus reflects the overall activity of this meganuclease on this particular site. Thus, meganucleases designed to cleave NANOG2 and NANOG4 DNA targets were analyzed for their ability to induce mutagenesis at their cognate endogenous site.

Single Chain I-CreI variants targeting respectively NANOG2 and NANOG4 targets were cloned in the pCLS1853 plasmid. The resulting plasmids, respectively pCLS4415, pCLS4416, pCLS4417, pCLS4418, pCLS4421 and pCLS4422 were used for this experiment. The day of previous experiments, cells from the human embryonic kidney cell line, 293-H (Invitrogen) were seeded in a 10 cm dish at density of 1×10⁶ cells/dish. The following day, cells were transfected with 10 μg of total DNA corresponding to the combination of an empty plasmid with a meganuclease-expressing plasmid using lipofectamine (Invitrogen). Plasmid ratio (empty/meganuclease plasmid) used were 10 μg/0 μg, 9 μg/1 μg, 5 μg/5 μg 0 μg/10 μg. 48 hours after transfection, cells were collected and diluted (dilution 1/20) in fresh culture medium. After 7 days of culture, cells were collected and genomic DNA extracted. 300 ng of genomic DNA were used to amplify the endogenous locus surrounding the meganuclease cleavage site by PCR amplification.

A DNA fragment surrounding each target NANOG target was amplified specifically. The specific PCR primers couples are:

A (NANOG2-fwd;
5′-CATGGATCTGCTTATTCAGGAC-3′;, SEQ ID NO: 59
B (NANOG2-rev;
5′-AGAGGCGATGTACGGACACATA-3′;, SEQ ID NO: 60)
and
C (NANOG4-fwd;
5′-ACCTGTGCTAGTACTCATGCTT-3′;, SEQ ID NO: 61)
D (NANOG4-rev;
5′-CTTGATCTCAGGGTTGAGGCTG-3′;, SEQ ID NO: 62)

that were used to amplify fragments surrounding respectively to NANOG2 (357 bp) and NANOG4 (381 bp).

PCR amplification was performed to obtain a fragment flanked by specific adaptator sequences (SEQ ID NO 63; 5′-CCATCTCATCCCTGCGTGTCTCCGACTCAG-3′ and SEQ ID NO 64; 5′-CCTATCCCCTGTGTGCCTTGGCAGTCTCAG-3′) provided by the company offering sequencing service (GATC Biotech AG, Germany) on the 454 sequencing system (454 Life Sciences). An average of 18,000 sequences was obtained from pools of 2 amplicons (500 ng each). After sequencing, different samples were identified based on barcode sequences introduced in the first of the above adaptators.

Sequences were then analyzed for the presence of insertions or deletions events (InDel) in the cleavage site of each NANOG target. Results are summarized in table IV.

InDel events could be detected in cells transfected with plasmids expressing Single Chain I-CreI variants meganucleases targeting respectively NANOG2 and NANOG4. Finally, the single Chain I-CreI variants pCLS4418 (SEQ ID NO: 31 encoded in plasmid SEQ ID NO: 47) targeting NANOG2 and pCLS4421 (SEQ ID NO: 34 encoded in plasmid SEQ ID NO: 50) targeting NANOG4 at the conditions 5 μg/5 μg show the highest activity at its endogenous locus as 0.317% and 0.323 of InDel events could be detected among the PCR fragment population, respectively.

TABLE IV
Mutagenesis by meganucleases targeting the NANOG gene

| Encoded | | InDel (%) | InDel (%) | InDel (%) |
|---|---|---|---|---|
| Meganucleases | pCLS | 1 μg | 5 μg | 10 μg |
| NANOG2 | 4415 | 0.099 | 0.276 | |
| (( )) | SEQ ID N^{o} 44 | | | |
| | 4416 | | 0.222 | 0.158 |
| | SEQ ID N^{o} 45 | | | |
| | 4417 | | | |
| | SEQ ID N^{o} 46 | | | |
| | 4418 | 0.115 | 0.317 | 0.09 |
| | SEQ ID N^{o} 47 | | | |
| NANOG4 | 4421 | | 0.323 | 0.139 |
| (0.027) | SEQ ID N^{o} 50 | | | |
| | 4422 | 0.086 | 0.11 | 0.097 |
| | SEQ ID N^{o} 51 | | | |

Legend to Table IV: 6 meganucleases were engineered to cleave 2 different DNA sequences respectively NANOG2 and NANOG4 within the NANOG gene. pCLS intends plasmid identification and corresponding SEQ ID NO. InDel intends meganuclease-induced mutagenesis determined by deep sequencing analysis of amplicons surrounding a specific target regarding the meganuclease plasmid quantity (data have been normalized for the cell plating efficiency). Values between brackets represent the sequencing background level.

Similar experiments were done for NANOG4 in iPS cells. Instead of pCLS4421, the plasmid used is pEF1a-4421 (SEQ ID NO: 84) carrying the same single chain meganuclease cloned under EF1a promoter for expression in iPS cells.

The day of transfection, iPS cells (Roger Hallar, Mount Sinai institute) were treated with 10 μM of ROCKi (Sigma) prior to be detach by CDK treatment. Then cells were counted and 1×10⁶ of cells/conditions was tranfected by nucleofection using the Amaxa nucleofector (Lonza) according to the stem cells nucleofection kit using the solution 2 and B16 program. Plasmid ratio (empty/meganuclease plasmid) used were 10 μg/5 μg, 15 μg/0 μg, 0 μg/15 μg.

Post-transfection cells were seeded in one well of 6-well plates on Geltrex (Invitrogen) coated dishes in conditioned medium (from feeder cells maintained in iPS medium) supplemented with 10 ng/ml of FGF2 (Invitrogen).

After 2, 3 and 7 days of culture, cells were collected and genomic DNA extracted.

As previously described for 293H cells, 300 ng of genomic DNA were used to amplify the endogenous locus surrounding the meganuclease cleavage site by PCR amplification using PCR primers couples C(NANOG4-fwd) (SEQ ID NO: 61) and D (NANOG4-rev) (SEQ ID NO: 62).

PCR amplification was performed to obtain a fragment flanked by specific adaptator sequences (SEQ ID NO 63; 5′-CCATCTCATCCCTGCGTGTCTCCGACTCAG-3′ and SEQ ID NO 64; 5′-CCTATCCCCTGTGTGCCTTGGCAGTCTCAG-3′) provided by the company offering sequencing service (GATC Biotech AG, Germany) on the 454 sequencing system (454 Life Sciences). An average of 18,000 sequences was obtained from pools of 2 amplicons (500 ng each). After sequencing, different samples were identified based on barcode sequences introduced in the first of the above adaptators.

Sequences were then analyzed for the presence of insertions or deletions events (InDel) in the cleavage site of each NANOG target. Results are summarized in table V.

InDel events could be detected in cells transfected with plasmids expressing Single Chain I-CreI variants meganucleases targeting NANOG4. Finally, the single Chain I-CreI pEF1a-4421 (SEQ ID NO: 84) targeting NANOG4 at the condition 15 μg show the highest activity at its endogenous locus as 0.503% of InDel events could be detected among the PCR fragment population, respectively.

TABLE V
Mutagenesis by meganucleases targeting the NANOG gene in iPS cells

| Encoded | | | InDel (%) | InDel (%) |
|---|---|---|---|---|
| Meganucleases | Plasmid | Days | 10 μg | 15 μg |
| NANOG4 | pEF1a-4421 | Day 2 | 0.405 | 0.503 |
| (0.021) | | Day 3 | 0.326 | 0.591 |
| | | Day 7 | 0.280 | 0.389 |

### Example 5

### NANOG Meganucleases Expression in Different Cell Types

Efficiency of meganucleases will depend of their expression level in the cells in fact if the meganuclease is not express for any reason in cell knock-in or NHEJ experiment could not be performed. Therefore to be validated, the different isoforms of meganucleases targeting the Nanog gene (NANOG2 and NANOG4) have been evaluated for their expression level in human embryonic kidney cell line 293H.

Single Chain I-CreI variants targeting respectively NANOG2 and NANOG4 targets were cloned in the pCLS1853 plasmid. The resulting plasmids, respectively pCLS4415, pCLS4416, pCLS4417, pCLS4418, pCLS4421 and pCLS4422 were used for this experiment. The day of previous experiments, cells from the human embryonic kidney cell line, 293-H (Invitrogen) were seeded in a 10 cm dish at density of 1×10⁶ cells/dish. The following day, cells were transfected with 10 μg of total DNA corresponding to the combination of an empty plasmid with a meganuclease-expressing plasmid using lipofectamine (Invitrogen). Plasmid ratio (empty/meganuclease plasmid) used were 10 μg/0 μg, 9 μg/1 μg, 5 μg/5 μg 0 μg/10 μg. 48 hours after transfection, cells were collected for protein extraction.

Cells were lysed in RIPA buffer with protease inhibitors (Santa Cruz) and protein supernatant was quantified by BCA quantification (Pierce). Then 20 μg/condition of protein was load on Precast Polyacrylamide Gels for protein separation. Protein was transferred to nitrocellulose membrane for blotting with the rabbit polyclonal anti-1-Cre I N75 antibody which recognize I-CRE1_derived custom meganucleases (1/20000). Revelation was made using a goat anti-rabbit IgG-HRP secondary antibody (1/5000) followed by incubation with Chemiluminescence Luminol Reagent. Then membrane was exposed to x-ray film.

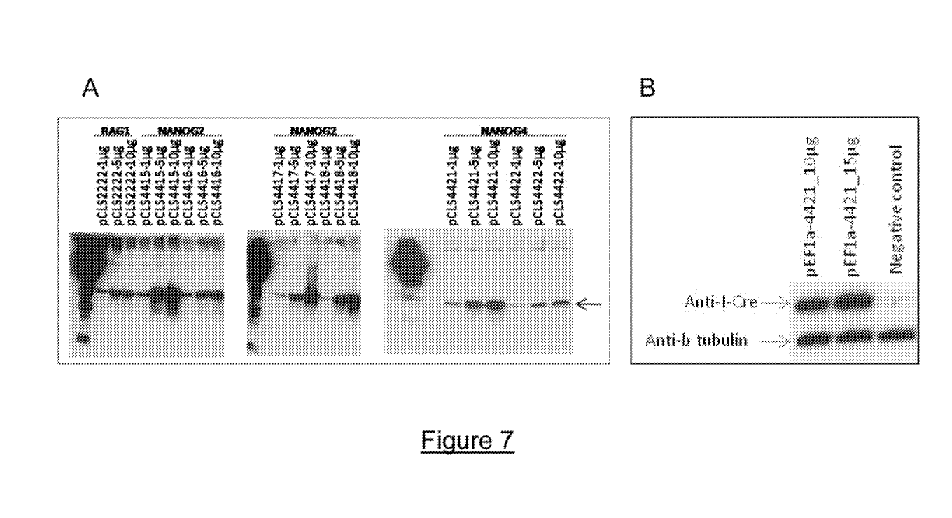

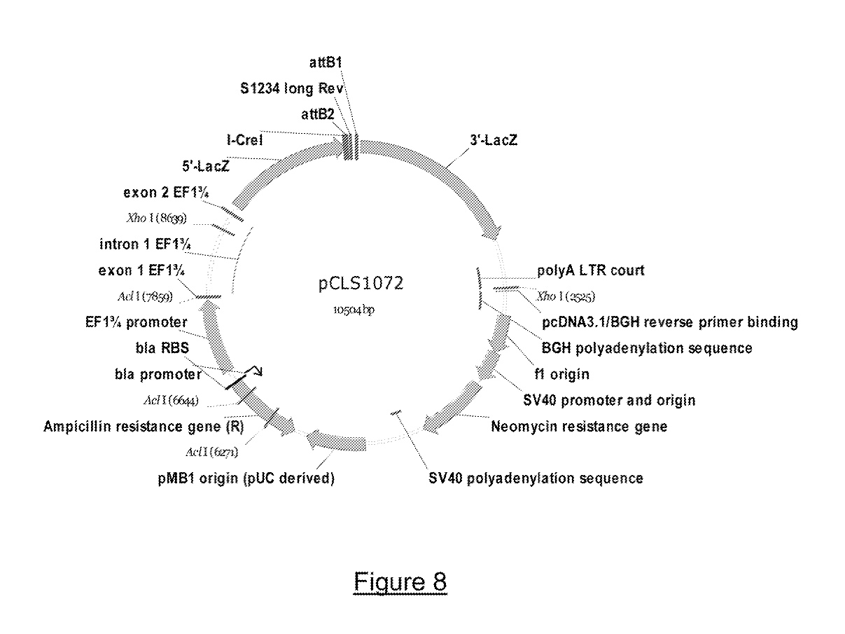

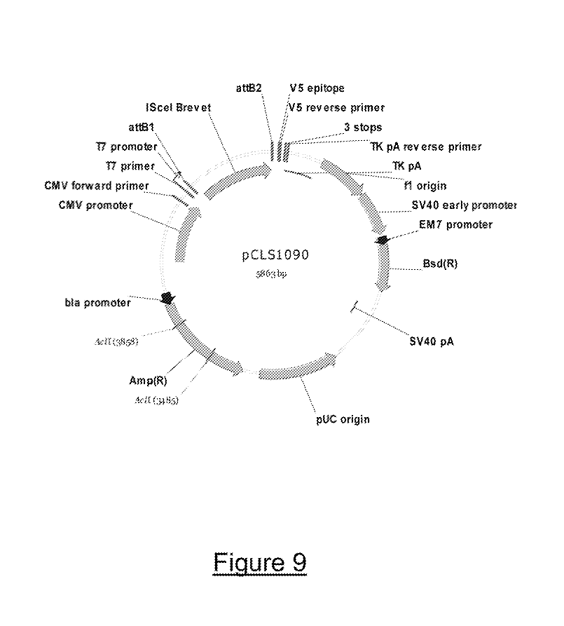

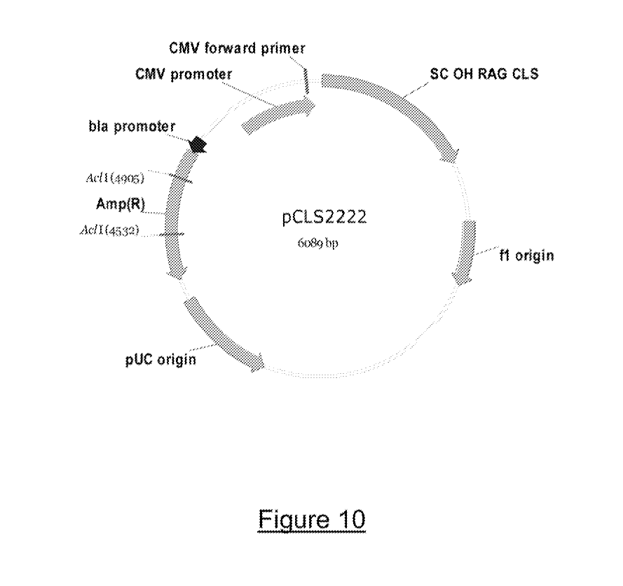

Results are shown in FIG. 7 panel A. All NANOG meganucleases are expressed in 293H cells and their level of expression increases with the quantity of meganucleases-expressing plasmids.

According to the same process NANOG4 meganuclease expression in iPS cells was also assessed using pEF1a-4421 (SEQ ID NO: 84).

The day of transfection, iPS cells were treated with 10 μM of ROCKi (Sigma) prior to be detached by CDK treatment. Then cells were counted and 1×10⁶ of cells/conditions was tranfected by nucleofection using the Amaxa nucleofector (Lonza) according to the stem cells nucleofection kit using the solution 2 and B16 program. Plasmid ratio (empty/meganuclease plasmid) used were 10 μg/5 μg, 15 μg/0 μg, 0 μg/15 μg.

Post-transfection cells were seeded in one well of 6-well plates on Geltrex (Invitrogen) coated dishes in conditioned medium (from feeder cells maintained in iPS medium) supplemented with 10 ng/ml of FGF2 (Invitrogen).

After 48 h days of culture, cells were collected for protein extraction. Cells were lysed in RIPA buffer with protease inhibitors (Santa Cruz) and protein supernatant was quantified by BCA quantification (Pierce). Then 20 μg/condition of protein was load on Precast Polyacrylamide Gels for protein separation. Protein was transferred to nitrocellulose membrane for blotting with the mouse monoclonal anti-1-Cre I N75 antibody which recognize I-CRE1_derived custom meganucleases (1/600). Revelation was made using a goat anti-mouse IgG-HRP secondary antibody (1/5000) followed by incubation with Chemiluminescence Luminol Reagent. Then membrane was exposed to x-ray film.

Results are shown in FIG. 7 panel B. NANOG4 meganuclease is expressed in iPS cells and its level of expression increases with the quantity of meganucleases-expressing plasmids.

### Example 6

### Generation of Clean iPS Cells

The process to generate clean iPS cells consists to first introduce the reprogramming transcription factors (OCT4, KLF4, SOX2 +/− C-MYC) using endonuclease in order to allow the reprogramming of somatic cells into iPS cells and second, to remove in the generated iPS cells the transgene using also meganuclease to obtain “clean” iPS cells.

### Example 6A

### “Pop Out” Strategy Validation in 293H Cells

This strategy has been first validated in 293H cells at endogenous RAG1 locus using single-chain RAG1 meganuclease (SC_RAG1) (pCLS2222, SEQ ID NO: 85).

The day of previous experiments, cells from the human embryonic kidney cell line, 293-H (Invitrogen) were seeded in a 10 cm dish at density of 1×10⁶ cells/dish. The following day, cells were transfected with 5 μg of total DNA corresponding to the combination of 3 μg 3F-matrix plasmid with 2 μg of meganuclease-expressing plasmid (pCLS2222, SEQ ID NO: 85) using lipofectamine (Invitrogen).

3 days after transfection, cells were collected and diluted (dilution 2000 cells/10 cm dishes) in fresh culture medium. After 10 days of culture, Neomycin selection (0.4 mg/ml) was added to the culture medium. At day 17, Neomycin resistance were picked and seeded into 96-well plate (one clone/well). At Day 22, plates were duplicated. One plate was stopped for PCR screen to identify targeted events (KI, Knock-in) and the second frozen for further analysis of KI positive clones.

The specific PCR primers couples used for the PCR screen are:

E (PCR-screen-KI3-F6:
5′-GGAGGATTGGGAAGACAATAGC-3′;, SEQ ID NO: 86)
F (Rag Ex2 R12:
5′-CTTTCACAGTCCTGTACATCTTGT-3′;. SEQ ID NO: 87)

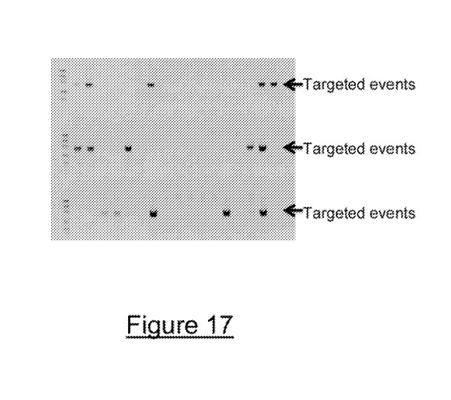

Primer E is located on the transgene whereas prime F is located on the endogenous targeted locus by the meganuclease thus only targeted events are be amplified. Examples of targeted events are shown in FIG. 17.

The results of the PCR screen showed that among neomycin resistant clones, 11.6% shown targeted integrations.

To validate this result and to identify clones with only targeted integration (absence of random integration), southern blot experiment was performed. 15 positive clones were selected and then amplified to obtain confluent 10 cm dishes. Genomic DNA was then extracted and digested by EcoRV. Then southern blot was performed using the “neo” probe of SEQ ID NO: 88.

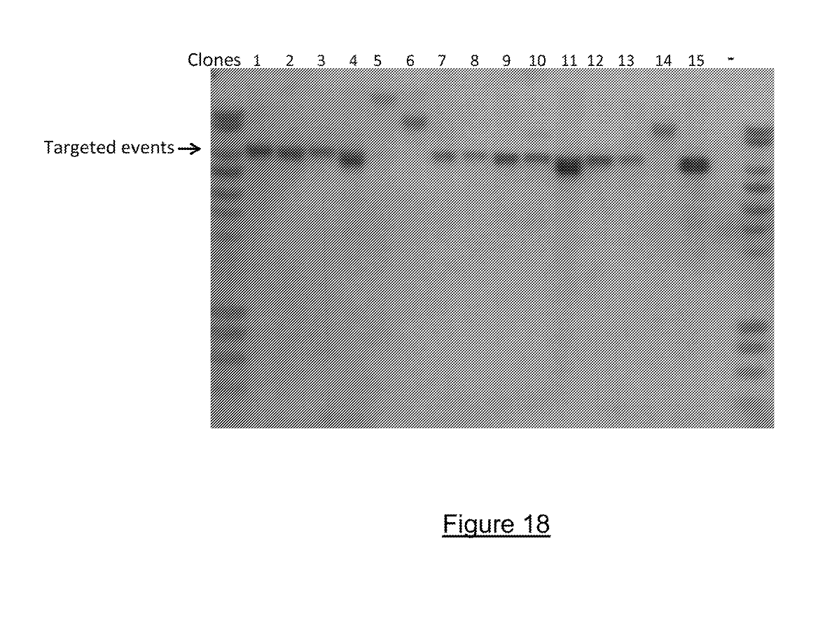

As shown in FIG. 18, among the 15 clones, 11 present unique targeted integration (clones 1, 2, 3, 4, 7, 8, 9, 11, 12, 13 and 15).

One clone was then chosen for “pop out” experiments to remove the transgene using I-Sce1 meganuclease (vector encoding I-SceI=pCLS1399, SEQ ID NO: 89). In fact, the 3F-matrix has been designed to carry two I-Sce1 sites (one following the 5′ homology and the second upstream the 3′ homology). Moreover, upstream the 3′ homology, the end of the 5′ homology has been added. This permits to remove the transgene without scar when the meganuclease I-Sce1 is expressed.

The day of previous experiments, cells from the selected clone, were seeded in a 10 cm dish at density of 1×10⁶ cells/dish. The following day, cells were transfected with 6 μg of meganuclease-expressing plasmid (pCLS1399, SEQ ID NO: 89) using lipofectamine (Invitrogen).

3 days after transfection, cells were collected and diluted (dilution 2000 cells/10 cm dishes) in fresh culture medium. At day 13, clones were picked and seeded into 96-well plate (one clone/well). At Day 21, plates were duplicated. One plate was stopped for PCR screen to identify “pop out events” and the second frozen for further analysis by sequencing.

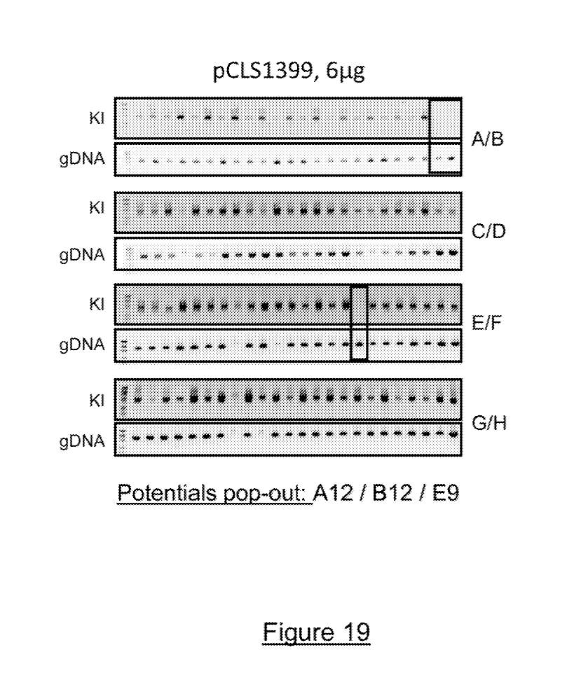

The same PCR as for KI event detection was used to identify the lost of targeted integration; in this case no amplification by primers E and F is observed. Examples of loss of targeted events are shown in FIG. 19.

“Pop out” candidates events were detected. Positives clones were then sent for sequencing analysis to confirm the excision of the transgene. Thanks to this methodology clear “popout” events were validated.

### Example 6B

### Generation of “Clean” iPS Cells

The strategy validated in 293H cells was applied to generate “clean” iPS cells from fibroblast cells.

The day of transfection, fibroblast cells are detached, counted and then transfected by electroporation of 1×10⁶ of cells/conditions using Amaxa nucleofector (Lonza, Kit NHDF, program U20) or Cytopulse technology (Cellectis, T4 solution). Several plasmid ratios (reprogramming matrix plasmid/meganuclease plasmid) are assessed to identify the best condition in order to obtain high rate of targeted events. The meganuclease plasmid is delivered either as DNA or RNA.

All transfected cells are then plated in a well of a 6-wells plate format in fibroblast medium. Day 3 post transfection cells are trypsinised and plated on 10 cm coated dishes (Geltex, Invitrogen or Gelatin, Sigma or Matrigel, BD Biosciences). At Day 5, fibroblast medium is replaced by conditioned iPS medium (from feeder cells maintained in iPS medium) with or without antibiotic selection (until selection is efficient) and Acid valproic for 8 days (Cambrex).

Cells are then maintained in conditioned iPS medium until iPS clones appeared. When clones reach a define size they are picked and replate into a new dish, one clone/dish. Then iPS clones are amplified in order to be characterized for their iPS status but also to identify iPS generated from a unique targeted integration event at the targeted locus.

True iPS clones containing only one unique targeted integration are then transfected with I-Sce1 meganuclease to achieve the “pop out” of the transgene.

The day of transfection, iPS cells are treated with 10 μM of ROCKi (Sigma) prior to be detached by CDK treatment. Then cells are counted and 1×10⁶ of cells/conditions is tranfected by nucleofection using the Amaxa nucleofector (Lonza) according to the stem cells nucleofection kit using the solution 2 and B16 program. A range of meganuclease plasmid quantity is used to identify the best condition to achieve high rate of “pop-out” events.

Cells are then seeded at clonal density into 10 cm dishes coated with Geltrex (Invitrogen) in conditioned medium (from feeder cells maintained in iPS medium) supplemented with 10 ng/ml of FGF2 (Invitrogen). Clones are then picked when they reach a define size then amplify to perform PCR screen to identify “pop out events” and to make a frozen stock for further analysis by sequencing.

PCR and sequencing analysis validate “clean” iPS cells.

### Example 7

### KO of NANOG by KI Using NANOG4 Meganuclease

Using the different NANOG endonucleases, different strategies can be applied to generate “safe” and “secure” iPS cells. Notably, the NANOG4 meganuclease targeting the intron 1 of NANOG gene can be used to delete the exon1 of NANOG using knock-in matrix. Our approach is to use this meganuclease to replace the exon1 of NANOG by a reporter gene which facilitates the identification of targeted events since its expression under NANOG4 regulatory elements.

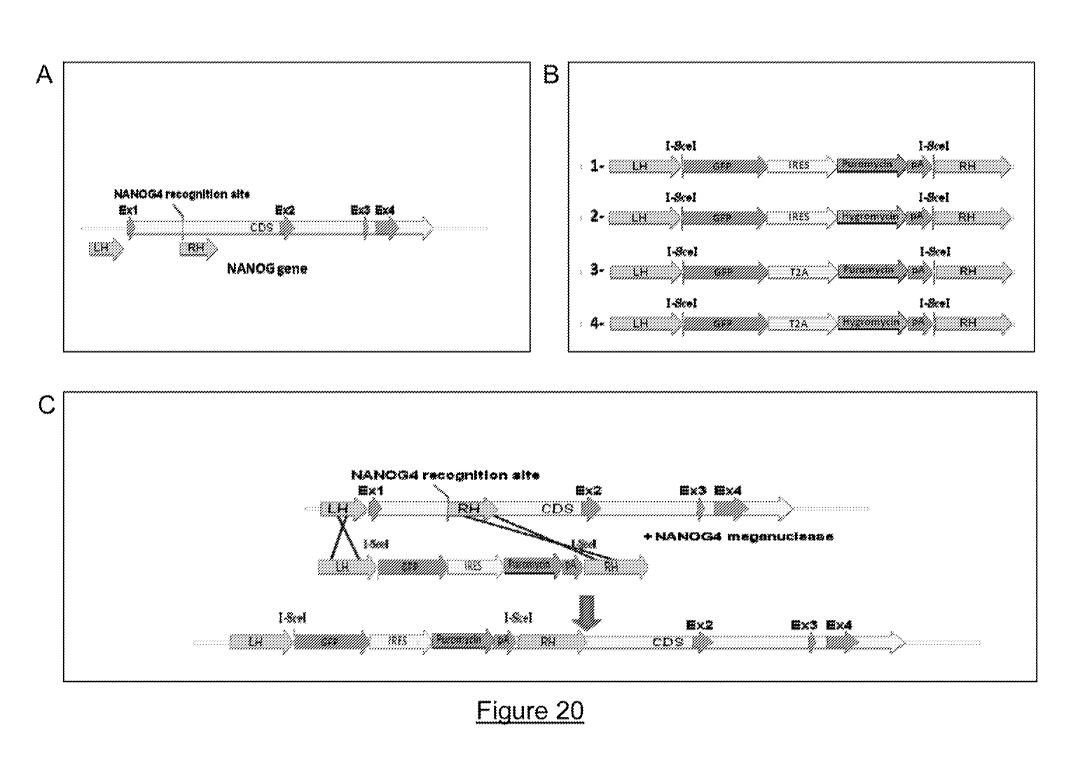

In order to replace exon1 by the reporter gene through meganuclease-mediated homologous recombination, in the recombination matrix, the left homology is homologous to the 5′ sequence before the exon1 and the right homology is homologous to the 3′ part just after the NANOG4 recognition site (FIG. 20 panel A). The matrices to achieve NANOG Knock Out (KO) are based on the same scaffold and are composed by (FIG. 20 panel B):
a reporter gene encoding for a fluorescent protein (GFP) for which expression is controlled by endogenous NANOG regulatory elements;
IRES or T2A proteolytic site to allow the expression of the resistance gene under endogenous NANOG regulatory elements;
a selection cassette: hygromycin or puromycin to select targeted events and to perform NANOG double KO;
two I-sce1 sites to remove the transgene using I-Sce 1 meganuclease.

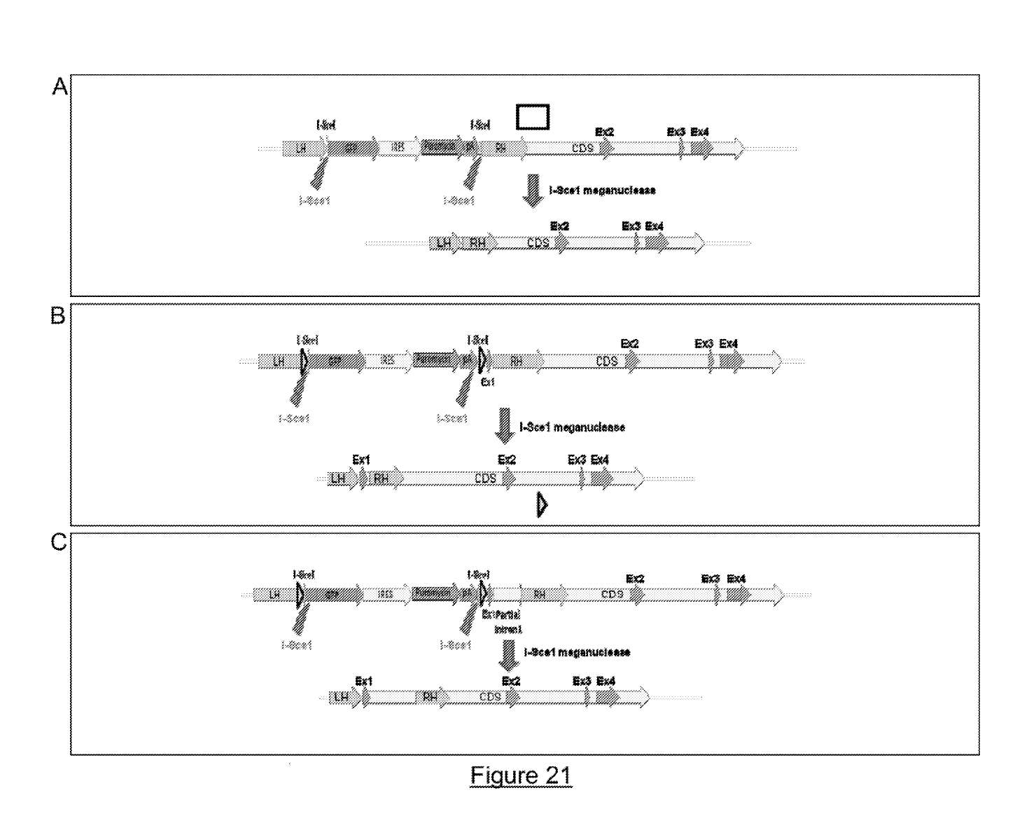

To mediate excision, different versions of the right homology (RH) have been designed (see FIG. 21).

The result of meganuclease-mediated homologous recombination is presented in FIG. 20 C.

As mentioned previously, two I-Sce1 sites were added in order to be able to remove the transgene from the NANOG knock-out iPS cells. For this, three different types of matrix were designed to generate irreversible, reversible or clean reversible KO of NANOG (respectively, FIG. 21A, B and C).

The first matrix (FIG. 21A), is composed by a classic left and right homology which leads to the deletion of NANOG exon1 and a part of intron 1 after I-Sce1 excision; thus the iPS cells obtained are irreversible KO for NANOG and fully secured and safe.

The two other matrices allow the reversion of the NANOG KO. In fact, in the second matrix as described in FIG. 21B, the end part of the left homology (direct repeat) is added before the right homology, as the NANOG exon1 to keep the KI Nanog allele functional after I-Sce1 transgene excision.

Finally, the third matrix is similar to the second with the addition of the part of the intron1 present before the NANOG4 recognition site which permits the excision of the transgene without any scar in the NANOG gene (FIG. 21 C).

These matrice are then used to generate “safe” and “secure” iPS cells according to the following process:

The day of transfection, iPS cells are treated with 10 μM of ROCKi (Sigma) prior to be detached by CDK treatment. Cells are then counted and 1×10⁶ of cells/conditions are tranfected by nucleofection using the Amaxa nucleofector (Lonza) according to the stem cells nucleofection kit using the solution 2 and B16 program. Several plasmid ratios (matrix plasmid/meganuclease plasmid) are assessed to identify the best condition in order to obtain high rate of targeted events.

Cells are then seeded into 10 cm dishes coated with Geltrex (Invitrogen) in conditioned medium (from feeder cells maintained in iPS medium) supplemented with 10 ng/ml of FGF2 (Invitrogen). The adapted selection is applied and then resistant clones are isolated and plated into 96-well plates. When cells reach confluence, plates are duplicated, one used to identify positive clones for targeted integration by PCR screen using primer allowing the amplification of both the endogenous locus and the transgene. Positive clones arev then next validated by southern blot experiments to confirm unique targeted integration.

Since clones probably show mono-allelic integrations, the same experiment is repeated on the positive clones using a matrix carrying a different selection that the one used for the generation of the first clones. Thus, cells resistant for both selections have both NANOG allele targeted. Data are validated by PCR and southern blot experiments.

Depending of the matrix used, the KO of NANOG gene is reversible or irreversible as described previously.

Matrices used are listed in the table below:

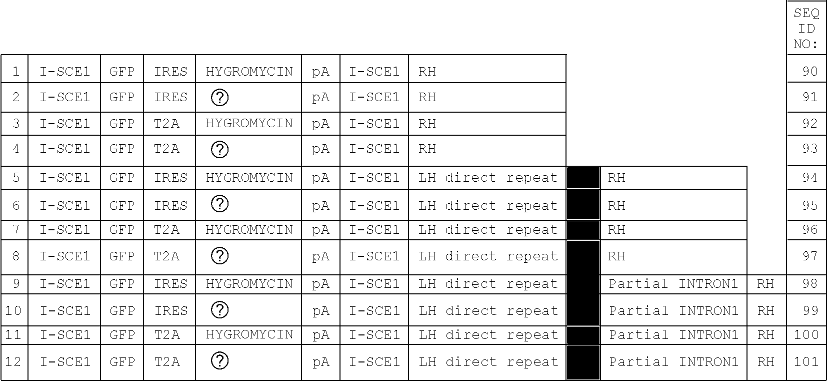

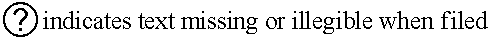

### MODIFICATIONS AND OTHER EMBODIMENTS

Various modifications and variations of the described meganuclease products, compositions and methods as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the medical, biological, chemical or pharmacological arts or related fields are intended to be within the scope of the following claims.

The present invention also concerns the CNCM (Collection Nationale de Cultures de Microorganismes, Institut Pasteur, Paris) deposits n° CNCM 1-4336 and CNCM 1-4337 as well as the inserts respectively encoding NANOG2 and NANOG4 variants (respectively SEQ ID NO: 30 and SEQ ID NO: 35) in the plasmids deposited under the respective deposit numbers above.

Unless specifically defined herein below, all technical and scientific terms used herein have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

### LIST OF REFERENCES CITED IN THE DESCRIPTION

1. Yu J, Vodyanik M A, Smuga-Otto K, Antosiewicz-Bourget J, Frane J L, Tian S, Nie J, Jonsdottir G A, Ruotti V, Stewart R, Slukvin, II, Thomson J A. Science 2007; 318: 1917-1920.
2. Takahashi K, Tanabe K, Ohnuki M, Narita M, Ichisaka T, Tomoda K, Yamanaka S. Cell 2007; 131: 861-872.
3. Takahashi K, Yamanaka S. Cell 2006; 126: 663-676.
4. Chambers I, Colby D, Robertson M, Nichols J, Lee S, Tweedie S, Smith A. Cell. 2003 May 30; 113(5):643-55.
5. Silva J, Nichols J, Theunissen T W, Guo G, van Oosten A L, Barrandon O, Wray J, Yamanaka S, Chambers I, Smith A. Cell. 2009 Aug. 21; 138(4):722-37.
6. Silva J, Barrandon O, Nichols J, Kawaguchi J, Theunissen T W, Smith A. PLoS Biol. 2008 Oct. 21; 6(10):e253.
7. Darr H, Mayshar Y, Benvenisty N. Development. 2006 March; 133(6):1193-201.
8. Dan H, Benvenisty N. Handb Exp Pharmacol. 2006; (174):1-19. Review.
9. Li J, Pan G, Cui K, Liu Y, Xu S, Pei D. J Biol. Chem. 2007 Jul. 6; 282(27):19481-92. Epub 2007 May 15.
10. Capecchi M R. Science. 1989 Jun. 16; 244(4910):1288-92. Review.
11. Smithies et al. Nat Med 2001 7(10): 1083-6
12. Thierry and Dujon Nucleic Acids Res 1992 20: 5625-5631
13. Puchta et al. Nucleic Acids Res 1993 21: 5034-5040
14. Rouet et al. Mol Cell Biol 1994 14: 8096-8106
15. Choulika et al. Mol Cell Biol 1995 15: 1968-1973
16. Puchta et al. Proc Natl Acad Sci U.S.A 1996 93: 5055-5060
17. Sargent et al. Mol Cell Biol 1997 17: 267-277
18. Cohen-Tannoudji et al. Mol Cell Biol 1998 18: 1444-1448
19. Donoho et al. Mol Cell Biol 1998 18: 4070-4078
20. Elliott et al. Mol Cell Biol 1998 18: 93-101
21. Chevalier and Stoddard Nucleic Acids Res 2001 29: 3757-3774
22. Smith et al. Nucleic Acids Res 1999 27: 674-681
23. Bibikova et al. Mol Cell Biol 2001 21: 289-297
24. Bibikova et al. Genetics 2002 161: 1169-1175
25. Bibikova et al. Science 2003 300: 764
26. Porteus and Baltimore Science 2003 300: 763
27. Alwin et al. Mol Ther 2005 12: 610-617
28. Urnov et al. Nature 2005 435: 646-651
29. Porteus M. H. Mol Ther 2006 13: 438-446
30. Pabo et al. Annu Rev Biochem 2001 70: 313-340
31. Jamieson et al. Nat Rev Drug Discov 2003 2: 361-368
32. Rebar and Pabo Science 1994 263: 671-673
33. Kim and Pabo Proc Natl Acad Sci USA 1998 95: 2812-2817
34. Klug et al. Proc Natl Acad Sci USA 1994 91: 11163-11167
35. Isalan and Klug Nat Biotechnol 2001 19: 656-660
36. Catto et al. Nucleic Acids Res 2006 34: 1711-1720
37. Hockemeyer et al., Nat. Biotechnol. 2009 September; 27(9): 851-7).
38. Chevalier et al. Nat Struct Biol 2001 8: 312-316
39. Chevalier et al. J Mol Biol 2003 329: 253-269
40. Moure et al. J Mol Biol 2003 334: 685-693,
41. Silva et al. J Mol Biol 1999 286: 1123-1136
42. Bolduc et al. Genes Dev 2003 17: 2875-2888
43. Ichiyanagi et al. J Mol Biol 2000 300: 889-901
44. Moure et al. Nat Struct Biol 2002 9: 764-770
45. Chevalier et al. Mol Cell 2002 10: 895-905
46. Epinat et al. Nucleic Acids Res 2003 31: 2952-62
47. Seligman et al. Genetics 1997 147: 1653-1664
48. Sussman et al. J Mol Biol 2004 342: 31-41
49. Arnould et al. J Mol Biol 2006 355: 443-458
50. Rosen et al. Nucleic Acids Res 2006 34: 4791-4800
51. Smith et al. Nucleic Acids Res 2006 34 e149
52. Doyon et al. J Am Chem Soc 2006 128: 2477-2484
53. Gimble et al. J Mol Biol 2003 334: 993-1008
54. Ashworth et al. Nature 2006 441: 656-659
55. Argast et al. J Mol Biol 1998 280: 345-353
56. Jurica et al. Mol Cell 1998 2: 469-476
57. Chevalier et al. Biochemistry 2004 43: 14015-14026
58. Pâques F. and Duchateau P., Current Gene Therapy, 2007, 7, 49-66
59. Aubry L, Bugi A, Lefort N, Rousseau F, Peschanski M, Perrier A L. PNAS. 2008 Oct. 28; 105(43):16707-12. Epub 2008 Oct. 15
60. Tabar V, Panagiotakos G, Greenberg E D, Chan B K, Sadelain M, Gutin P H, Studer L. Nat. Biotechnol. 2005 May; 23(5):601-6. Epub 2005 Apr. 24.
61. Jeter C R, Badeaux M, Choy G, Chandra D, Patrawala L, Liu C, Calhoun-Davis T, Zaehres H, Daley G Q, Tang D G. Stem Cells. 2009 May; 27(5):993-1005.
62. Roy N S, Cleren C, Singh S K, Yang L, Beal M F, Goldman S A. Nat. Med. 2006 November; 12(11):1259-68. Epub 2006 Oct. 22. Erratum in: Nat. Med. 2007 March; 13(3):385.
63. You J S, Kang J K, Seo D W, Park J H, Park J W, Lee J C, Jeon Y J, Cho E J, Han J W. Cancer Res. 2009 Jul. 15; 69(14):5716-25. Epub 2009 Jun. 30.
64. Ji J, Werbowetski-Ogilvie T E, Zhong B, Hong S H, Bhatia M. PLoS One. 2009 Nov. 30; 4(11):e8065.
65. Ji L, Liu Y X, Yang C, Yue W, Shi S S, Bai C X, Xi J F, Nan X, Pei X T. J Cell Physiol. 2009 October; 221(1):54-66.
66. Brignier A C, Gewirtz A M. J Allergy Clin Immunol. 2010 February; 125(2 Suppl 2):S336-44. Epub 2010 January 12. Review.
67. Phillips B W, Crook J M. BioDrugs. 2010 Apr. 1; 24(2):99-108. doi: 10.2165/11532270-000000000-00000. Review.
68. Boch, J., H. Scholze, et al. (2009). “Breaking the code of DNA binding specificity of TAL-type III effectors.” Science 326(5959): 1509-12.
69. Capecchi, M. R. (1989). “Altering the genome by homologous recombination.” Science 244(4910): 1288-92.
70. Christian, M., T. Cermak, et al. (2010). “Targeting DNA double-strand breaks with TAL effector nucleases.” Genetics 186(2): 757-61.
71. Li, T., S. Huang, et al. (2010). “TAL nucleases (TALNs): hybrid proteins composed of TAL effectors and FokI DNA-cleavage domain.” Nucleic Acids Res 39(1): 359-72.
72. Moscou, M. J. and A. J. Bogdanove (2009). “A simple cipher governs DNA recognition by TAL effectors.” Science 326(5959): 1501.
73. Smithies, O. (2001). “Forty years with homologous recombination.” Nat Med 7(10): 1083-6.

1. A method for generating a secure iPS cell or a derivate thereof at various differentiation stages, the method comprising expressing at least one endonuclease in an iPS cell or a derivate thereof, wherein the at least one endonuclease induces a double-strand break in a NANOG gene to produce a cell lacking capacity for de-differentiation to a more pluripotent state.

2-3. (canceled)

4. The method according to claim 1, wherein said endonuclease is a meganuclease.

5. A meganuclease variant that induces a double-strand break in a NANOG gene.

6. The meganuclease of claim 5, which recognizes the NANOG4 sequence (SEQ ID NO: 18).

7. The meganuclease of claim 5, which recognizes the NANOG4 sequence (SEQ ID NO: 18) and which comprises a variant I-CreI amino acid sequence selected from the group consisting of SEQ ID NO: 33 to 40.

8-9. (canceled)

10. The meganuclease variant of claim 5, which is a homodimer, a heterodimer, or a single chain.

11-14. (canceled)

15. The polynucleotide that encodes the meganuclease of claim 5 or a fragment thereof having meganuclease activity.

16. (canceled)

17. A vector, comprising the polynucleotide of claim 15.

18. A host cell, comprising the vector of claim 17.

19-28. (canceled)

29. A cell bank, comprising cells in which NANOG is knocked-out by an endonuclease.

30. A cell bank, comprising cells in which NANOG is knocked-out by a meganuclease

31-34. (canceled)

35. A purified iPS cells culture, wherein a NANOG gene of said iPS cells is not functional.

36. A purified differentiated cell culture selected from the purified iPS cells culture according to claim 35.

37. The method according to claim 1, wherein said NANOG gene is knocked-out.

38. The method according to claim 1, further comprising introducing into the iPS cell or derivate thereof a targeting construct comprising sequences sharing homologies with regions surrounding a site of the double-strand break in the NANOG gene.

39. The method according to claim 1, wherein said endonuclease is a TALEN.

40. The meganuclease variant of claim 6, which is a homodimer, a heterodimer, or a single chain.

41. The meganuclease variant of claim 7, which is a homodimer, a heterodimer, or a single chain.

42. The polynucleotide that encodes the meganuclease of claim 6 or a fragment thereof having meganuclease activity.

43. The polynucleotide that encodes the meganuclease of claim 7 or a fragment thereof having meganuclease activity.

44. A vector, comprising the polynucleotide of claim 42.

45. A host cell, comprising the vector of claim 44.

46. A vector, comprising the polynucleotide of claim 43.

47. A host cell, comprising the vector of claim 46.


SEQUENCE LISTING (102 sequences)

SEQ ID NO: 1 - protein, 163 aa, Chlamydomonas reinhardtii
MNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 2 - DNA, 24 bp, Artificial Sequence
  Description of Artificial Sequence Synthetic C1221 target oligonucleotide
tcaaaacgtcgtacgacgttttga

SEQ ID NO: 3 - protein, 167 aa, Artificial Sequence
  Description of Artificial Sequence Synthetic I-CreI N75 polypeptide
MANTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSPAAD

SEQ ID NO: 4 - DNA, 24 bp, Artificial Sequence
  Description of Artificial Sequence Synthetic 10AAC_P target oligonucleotide
tcaacacgtcgtacgacgtgttga

SEQ ID NO: 5 - DNA, 24 bp, Artificial Sequence
  Description of Artificial Sequence Synthetic 5CCT_P target oligonucleotide
tcaaaaccctgtacagggttttga

SEQ ID NO: 6 - DNA, 24 bp, Artificial Sequence
  Description of Artificial Sequence Synthetic 10TAG_P target oligonucleotide
tctagacgtcgtacgacgtctaga

SEQ ID NO: 7 - DNA, 24 bp, Artificial Sequence
  Description of Artificial Sequence Synthetic 5GAG_P target oligonucleotide
tcaaaacgaggtacctcgttttga

SEQ ID NO: 8 - DNA, 24 bp, Artificial Sequence
  Description of Artificial Sequence Synthetic NANOG2.1 target oligonucleotide
ccaacatcctgaacctcagctaca

SEQ ID NO: 9 - DNA, 24 bp, Artificial Sequence
  Description of Artificial Sequence Synthetic NANOG2.2 target oligonucleotide
ccaacatcctgtacctcagctaca

SEQ ID NO: 10 - DNA, 24 bp, Artificial Sequence
  Description of Artificial Sequence Synthetic NANOG2.3 target oligonucleotide
ccaacatcctgtacaggatgttgg

SEQ ID NO: 11 - DNA, 24 bp, Artificial Sequence
  Description of Artificial Sequence Synthetic NANOG2.4 target oligonucleotide
tgtagctgaggtacctcagctaca

SEQ ID NO: 12 - DNA, 24 bp, Artificial Sequence
  Description of Artificial Sequence Synthetic NANOG2.5 target oligonucleotide
ccaacatcctgaacaggatgttgg

SEQ ID NO: 13 - DNA, 24 bp, Artificial Sequence
  Description of Artificial Sequence Synthetic NANOG2.6 target oligonucleotide
tgtagctgaggaacctcagctaca

SEQ ID NO: 14 - DNA, 24 bp, Artificial Sequence
  Description of Artificial Sequence Synthetic 10TGA_P target oligonucleotide
tctgaacgtcgtacgacgttcaga

SEQ ID NO: 15 - DNA, 24 bp, Artificial Sequence
  Description of Artificial Sequence Synthetic 5GCT_P target oligonucleotide
tcaaaacgctgtacagcgttttga

SEQ ID NO: 16 - DNA, 24 bp, Artificial Sequence
  Description of Artificial Sequence Synthetic 10AAG_P target oligonucleotide
tcaagacgtcgtacgacgtcttga

SEQ ID NO: 17 - DNA, 24 bp, Artificial Sequence
  Description of Artificial Sequence Synthetic 5ATT_P target oligonucleotide
tcaaaacattgtacaatgttttga

SEQ ID NO: 18 - DNA, 24 bp, Artificial Sequence
  Description of Artificial Sequence Synthetic NANOG4.1 target oligonucleotide
actgaacgctgtaaaatagcttaa

SEQ ID NO: 19 - DNA, 24 bp, Artificial Sequence
  Description of Artificial Sequence Synthetic NANOG4.2 target oligonucleotide
actgaacgctgtacaatagcttaa

SEQ ID NO: 20 - DNA, 24 bp, Artificial Sequence
  Description of Artificial Sequence Synthetic NANOG4.3 target oligonucleotide
actgaacgctgtacagcgttcagt

SEQ ID NO: 21 - DNA, 24 bp, Artificial Sequence
  Description of Artificial Sequence Synthetic NANOG4.4 target oligonucleotide
ttaagctattgtacaatagcttaa

SEQ ID NO: 22 - DNA, 24 bp, Artificial Sequence
  Description of Artificial Sequence Synthetic NANOG4.5 target oligonucleotide
actgaacgctgtaaagcgttcagt

SEQ ID NO: 23 - DNA, 24 bp, Artificial Sequence
  Description of Artificial Sequence Synthetic NANOG4.6 target oligonucleotide
ttaagctattgtaaaatagcttaa

SEQ ID NO: 24 - protein, 32 aa, Artificial Sequence
  Description of Artificial Sequence Synthetic RM2 peptidic linker polypeptide
AAGGSDKYNQALSKYNQALSKYNQALSGGGGS

SEQ ID NO: 25 - protein, 354 aa, Artificial Sequence
  Description of Artificial Sequence Synthetic SCOH-NANOG2-pCLS4412 polypeptide
MANTKYTEEFLLYLAGFVDGDGSIIAQIRPNQSRKFKHYLQLTFKVTQKTQRRWFLDKLVDEIGVGYVADGGSVSNYILSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQNCKFKHQLRLTFQVTQKTQRRWFLDKLVDRIGVGYVYDSGSVSYYYLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 26 - protein, 354 aa, Artificial Sequence
  Description of Artificial Sequence Synthetic SCOH-NANOG2-pCLS4413 polypeptide
MANTKYTEEFLLYLAGFVDGDGSIIAQIRPNQSRKFKHYLQLTFKVTQKTQRRWFLDKLVDEIGVGYVADGGSVSNYILSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQNCKFKHQLRLTFQVTQKTQRRWFLDKLVDRIGVGYVYDSGSVSYYYLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 27 - protein, 354 aa, Artificial Sequence
  Description of Artificial Sequence Synthetic SCOH-NANOG2-pCLS4414 polypeptide
MANTKYNEEFLLYLAGFVDGDGSIIAQIRPNQSRKFKHYLQLTFKVTQKTQRRWFLDKLVDEIGVGYVADGGSVSNYILSEIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDSDGSIIAQIKPHQSCKFKHQLRLTFYVTQKTQRRWFLDKLVDRIGVGYVYDSGSVSNYTLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 28 - protein, 354 aa, Artificial Sequence
  Description of Artificial Sequence Synthetic SCOH-NANOG2-pCLS4415 polypeptide
MANTKYNEEFLLYLAGFVDGDGSIIAQIRPNQSRKFKHYLQLTFKVTQKTQRRWFLDKLVDEIGVGYVADGGSVSNYILSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPHQSCKFKHQLRLTFYVTQKTQRRWFLDKLVDRIGVGYVYDSGSVSNYTLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 29 - protein, 354 aa, Artificial Sequence
  Description of Artificial Sequence Synthetic SCOH-NANOG2-pCLS4416 polypeptide
MANTKYNEEFLLYLAGFVDGDGSIIAQIRPNQSRKFKHYLQLTFKVTQKTQRRWFLDKLVDEIGVGYVADGGSVSNYILSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPHQSCKFKHQLRLTFYVTQKTQRRWFLDKLVDRIGVGYVYDSGSVSNYTLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 30 - protein, 354 aa, Artificial Sequence
  Description of Artificial Sequence Synthetic SCOH-NANOG2-pCLS4417 polypeptide
MANTKYNEEFLLYLAGFVDGDGSIIAQIRPNQSRKFKHYLQLTFKVTQKTQRRWILDKLVDEIGAGYVADGGSVSNYILSEIKPLHNFLTQLQPFLELKQKQANLVLKIIEQLPSAKESPDKFLEVCTWVDQIAALNDSKTRKTTSEAVRAVLDSLSEKKKSSPAAGGSDKYNQALSKYNQALSKYNQALSGGGGSNKKFLLYLAGFVDSDGSIIAQIKPHQSCKFKHQLRLTFYVTQKTQRRWFLDKLVDRIGVGYVYDSGSVSNYTLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 31 - protein, 354 aa, Artificial Sequence
  Description of Artificial Sequence Synthetic SCOH-NANOG2-pCLS4418 polypeptide
MANTKYNEEFLLYLAGFVDGDGSIIAQIRPNQSRKFKHYLQLTFKVTQKTQRRWILDKLVDEIGAGYVADGGSVSNYILSEIKPLHNFLTQLQPFLELKQKQANLVLKIIEQLPSAKESPDKFLEVCTWVDQVAALNDSKTRKTTSEAVRAVLDSLSEKKKSSPAAGGSDKYNQALSKYNQALSKYNQALSGGGGSNKKFLLYLAGFVDSDGSIIAQIKPHQSCKFKHQLRLTFYVTQKTQRRWFLDKLVDRIGVGYVYDSGSVSNYTLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 32 - protein, 354 aa, Artificial Sequence
  Description of Artificial Sequence Synthetic SCOH-NANOG2-pCLS4419 polypeptide
MANTKYNEEFLLYLAGFVDGDGSIIAQIRPNQSRKFKHYLQLTFKVTQKTQRRWILDKLVDEIGAGYVADGGSVSNYILSKIKPLHNFLTQLQPFLELKQKQANLVLKIIEQLPSAKESPDKFLEVCTWVDQVAALNDSKTRKTTSEAVRAVLDSLSEKKKSSPAAGGSDKYNQALSKYNQALSKYNQALSGGGGSNKKFLLYLAGFVDSDGSIIAQIKPHQSCKFKHQLRLTFYVTQKTQRRWFLDKLVDRIGVGYVYDSGSVSNYTLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 33 - protein, 354 aa, Artificial Sequence
  Description of Artificial Sequence Synthetic SCOH-NANOG4-pCLS4420 polypeptide
MANTKYNEEFLLYLAGFVDGDGSIIAQIKPNQSTKFKHRLQLTLYVTQKTQRRWCLDKLVDEIGVGYVEDSGSVSRYVLSEIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLLYLAGFVDSDGSIIAQIKPGQSYKFKHQLYLTFQVTQKTQRRWFLDKLVDRIGVGYVRDSGSVSNYILSEVKPLHNLLTQLQPFLKLKQKQANLVLKIIEQLPSAKESPDKFLEVCTWVDQIAALNDSKTRKTTSETVRAVLDSLSEKKKSSP

SEQ ID NO: 34 - protein, 354 aa, Artificial Sequence
  Description of Artificial Sequence Synthetic SCOH-NANOG4-pCLS4421 polypeptide
MANTKYNEEFLLYLAGFVDGDGSIIAQIKPNQSTKFKHRLQLTLYVTQKTQRRWCLDKLVDEIGVGYVEDSGSVSRYVLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPGQSYKFKHQLYLTFQVTQKTQRRWFLDKLVDRIGVGYVRDSGSVSNYILSEVKPLHNLLTQLQPFLKLKQKQANLVLKIIEQLPSAKESPDKFLEVCTWVDQVAALNDSKTRKTTSETVRAVLDSLSEKKKSSP

SEQ ID NO: 35 - protein, 354 aa, Artificial Sequence
  Description of Artificial Sequence Synthetic SCOH-NANOG4-pCLS4422 polypeptide
MANTKYNEEFLLYLAGFVDGDGSIIAQIKPNQSTKFKHRLQLTLYVTQKTQRRWCLDKLVDEIGVGYVEDSGSVSRYVLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPGQSYKFKHQLYLTFQVTQKTQRRWFLDKLVDRIGVGYVRDSGSVSNYILSEVKPLHNLLTQLQPFLKLKQKQANLVLKIIEQLPSAKESPDKFLEVCTWVDQVAALNDSKTRKTTSETVRAVLDSLSEKKKSSP

SEQ ID NO: 36 - protein, 354 aa, Artificial Sequence
  Description of Artificial Sequence Synthetic SCOH-NANOG4-pCLS4697 polypeptide
MANTKYNEEFLLYLAGFVDGDGSIIAQIKPNQSTKFKHRLQLTLYVTQKTQRRWCLDKLVDEIGVGYVEDSGSVSRYVLSEIKPLHNFLTQLQPFLELKQKQANLVLKIIEQLPSAKESPDKFLEVCTWVDQIAALNDSKTRKTTSETVRAVLDSLSEKKKSSPAAGGSDKYNQALSKYNQALSKYNQALSGGGGSNKKFLMYLAGFVDSDGSIIAQIKPNQSYKFKHQLYLTFQVTQKTQRRWFLDKLVDRIGVGYVRDSGSVSNYILSEIKPLHNFLTQLQPFLKLKQKQANLVLKIIEQLPSAKESPDKFLEVCTWVDQIAALNDSKTRKNTSETVRAVLDSLSEKKKSSP

SEQ ID NO: 37 - protein, 354 aa, Artificial Sequence
  Description of Artificial Sequence Synthetic SCOH-NANOG4-pCLS4698 polypeptide
MANTKYNEEFLLYLAGFVDGDGSIIAQIKPNQSTKFKHRLQLTLYVTQKTQRRWCLDKLVDEIGVGYVEDSGSVSRYVLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLMYLAGFVDSDGSIIAQIKPNQSYKFKHQLYLTFQVTQKTQRRWFLDKLVDRIGVGYVRDSGSVSNYILSEIKPLHNFLTQLQPFLKLKQKQANLVLKIIEQLPSAKESPDKFLEVCTWVDQVAALNDSKTRKNTSETVRAVLDSLSEKKKSSP

SEQ ID NO: 38 - protein, 354 aa, Artificial Sequence
  Description of Artificial Sequence Synthetic SCOH-NANOG4-pCLS4699 polypeptide
MANTKYNEEFLLYLAGFVDGDGSIIAQIKPNQSTKFKHRLQLTLYVTQKTQRRWCLDKLVDEIGVGYVEDSGSVSRYVLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLMYLAGFVDSDGSIIAQIKPNQSYKFKHQLYLTFQVTQKTQRRWFLDKLVDRIGVGYVRDSGSVSNYILSEIKPLHNFLTQLQPFLKLKQKQANLVLKIIEQLPSAKESPDKFLEVCTWVDQVAALNDSKTRKNTSETVRAVLDSLSEKKKSSP

SEQ ID NO: 39 - protein, 354 aa, Artificial Sequence
  Description of Artificial Sequence Synthetic SCOH-NANOG4-pCLS4701 polypeptide
MANTKYNEEFLLYLAGFVDGDGSIIAQIKPNQSTKFKHRLQLTLYVTQKTQRRWCLDKLVDEIGVGYVEDSGSVSRYVLSEIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPGQSYKFKHQLYLTFQVTQKTQRRWVLDKLVDRIGVGYVRDSGSVSNYILSEVKPLHNFLTQLQPFLKLKQKQANLVLKIIEQLPSAKESPDKFLEVCTWVDQVAALNDSKTRKTTSETVRAVLDSLSEKKKSSP

SEQ ID NO: 40 - protein, 354 aa, Artificial Sequence
  Description of Artificial Sequence Synthetic SCOH-NANOG4-pCLS4702 polypeptide
MANTKYNEEFLLYLAGFVDGDGSIIAQIKPNQSTKFKHRLQLTLYVTQKTQRRWCLDKLVDEIGVGYVEDSGSVSRYVLSKIKPLHNFLTQLQPFLELKQKQANLVLKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPGQSYKFKHQLYLTFQVTQKTQRRWVLDKLVDRIGVGYVRDSGSVSNYILSEVKPLHNFLTQLQPFLKLKQKQANLVLKIIEQLPSAKESPDKFLEVCTWVDQVAALNDSKTRKTTSETVRAVLDSLSEKKKSSP

SEQ ID NO: 41 - DNA, 6233 bp, Artificial Sequence
  Description of Artificial Sequence Synthetic pCLS4412 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataccgaagagttcctgctgtacctggccggctttgtggacggtgacggtagcatcatcgctcagattcgtccaaaccagtctcgtaagtttaaacattacctacagttgacctttaaagtgactcaaaagacccagcgccgttggtttctggacaaactagtggatgaaattggcgttggttacgtagctgatggtggtagcgtttccaactacatcttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccaaatcaaaactgtaagttcaaacaccagctccgtttgacctttcaagtcactcagaagacacaaagaaggtggttcttggacaaattggttgatcgtattggtgtgggctatgtctacgactctggctctgtgtcatactactacctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcagattgctgccttgaatgactccaagaccagaaaaaccacctctgagactgtgagggcagttctggatagcctctctgagaagaaaaagtcctctccttag

SEQ ID NO: 42 - DNA, 6233 bp, Artificial Sequence
  Description of Artificial Sequence Synthetic pCLS4413 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataccgaagagttcctgctgtacctggccggctttgtggacggtgacggtagcatcatcgctcagattcgtccaaaccagtctcgtaagtttaaacattacctacagttgacctttaaagtgactcaaaagacccagcgccgttggtttctggacaaactagtggatgaaattggcgttggttacgtagctgatggtggtagcgtttccaactacatcttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccaaatcaaaactgtaagttcaaacaccagctccgtttgacctttcaagtcactcagaagacacaaagaaggtggttcttggacaaattggttgatcgtattggtgtgggctatgtctacgactctggctctgtgtcatactactacctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttag

SEQ ID NO: 43 - DNA, 6233 bp, Artificial Sequence
  Description of Artificial Sequence Synthetic pCLS4414 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcatcgctcagattcgtccaaaccagtctcgtaagtttaaacattacctacagttgacctttaaagtgactcaaaagacccagcgccgttggtttctggacaaactagtggatgaaattggcgttggttacgtagctgatggtggtagcgtttccaactacatcttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccacatcaatcttgtaagttcaaacaccagctccgtttgaccttttacgtcactcagaagacacaaagaaggtggttcttggacaaattggttgatcgtattggtgtgggctatgtctacgactctggctctgtgtcaaactacaccctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcagattgctgccttgaatgactccaagaccagaaaaaccacctctgagactgtgagggcagttctggatagcctctctgagaagaaaaagtcctctccttag

SEQ ID NO: 44 - DNA, 6233 bp, Artificial Sequence
  Description of Artificial Sequence Synthetic pCLS4415 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcatcgctcagattcgtccaaaccagtctcgtaagtttaaacattacctacagttgacctttaaagtgactcaaaagacccagcgccgttggtttctggacaaactagtggatgaaattggcgttggttacgtagctgatggtggtagcgtttccaactacatcttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccacatcaatcttgtaagttcaaacaccagctccgtttgaccttttacgtcactcagaagacacaaagaaggtggttcttggacaaattggttgatcgtattggtgtgggctatgtctacgactctggctctgtgtcaaactacaccctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttag

SEQ ID NO: 45 - DNA, 6233 bp, Artificial Sequence
  Description of Artificial Sequence Synthetic pCLS4416 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcatcgctcagattcgtccaaaccagtctcgtaagtttaaacattacctacagttgacctttaaagtgactcaaaagacccagcgccgttggtttctggacaaactagtggatgaaattggcgttggttacgtagctgatggtggtagcgtttccaactacatcttaagcaaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccacatcaatcttgtaagttcaaacaccagctccgtttgaccttttacgtcactcagaagacacaaagaaggtggttcttggacaaattggttgatcgtattggtgtgggctatgtctacgactctggctctgtgtcaaactacaccctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttag

SEQ ID NO: 46 - DNA, 6233 bp, Artificial Sequence
  Description of Artificial Sequence Synthetic pCLS4417 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcatcgctcagattcgtccaaaccagtctcgtaagtttaaacattacctacagttgacctttaaagtgactcaaaagacccagcgccgttggattctggacaaactagtggatgaaattggcgctggttacgtagctgatggtggtagcgtttccaactacatcttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaagctgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccacatcaatcttgtaagttcaaacaccagctccgtttgaccttttacgtcactcagaagacacaaagaaggtggttcttggacaaattggttgatcgtattggtgtgggctatgtctacgactctggctctgtgtcaaactacaccctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcagattgctgccttgaatgactccaagaccagaaaaaccacctctgagactgtgagggcagttctggatagcctctctgagaagaaaaagtcctctccttag

SEQ ID NO: 47 - DNA, 6233 bp, Artificial Sequence
  Description of Artificial Sequence Synthetic pCLS4418 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcatcgctcagattcgtccaaaccagtctcgtaagtttaaacattacctacagttgacctttaaagtgactcaaaagacccagcgccgttggattctggacaaactagtggatgaaattggcgctggttacgtagctgatggtggtagcgtttccaactacatcttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaagctgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccacatcaatcttgtaagttcaaacaccagctccgtttgaccttttacgtcactcagaagacacaaagaaggtggttcttggacaaattggttgatcgtattggtgtgggctatgtctacgactctggctctgtgtcaaactacaccctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttag

SEQ ID NO: 48 - DNA, 6233 bp, Artificial Sequence
  Description of Artificial Sequence Synthetic pCLS4419 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcatcgctcagattcgtccaaaccagtctcgtaagtttaaacattacctacagttgacctttaaagtgactcaaaagacccagcgccgttggattctggacaaactagtggatgaaattggcgctggttacgtagctgatggtggtagcgtttccaactacatcttaagcaaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaagctgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccacatcaatcttgtaagttcaaacaccagctccgtttgaccttttacgtcactcagaagacacaaagaaggtggttcttggacaaattggttgatcgtattggtgtgggctatgtctacgactctggctctgtgtcaaactacaccctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttag

SEQ ID NO: 49 - DNA, 6233 bp, Artificial Sequence
  Description of Artificial Sequence Synthetic pCLS4420 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcatcgctcagattaaaccaaaccagtctaccaagtttaaacatcgtctacagttgaccctgtacgtgactcaaaagacccagcgccgttggtgtctggacaaactagtggatgaaattggcgttggttacgtagaagattctggtagcgtttcccgttacgttttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccaggtcaatcttacaagttcaaacaccagctctacttgacctttcaagtcactcagaagacacaaagaaggtggttcttggacaaattggttgatcgtattggtgtgggctatgtcagagactctggctctgtgtcaaactacatcctgtctgaagttaagcctcttcataacctgctcacccaactgcaacccttcttgaagctcaaacagaagcaagcaaatctggttttgaaaatcatcgagcaactgccatctgccaaggagtcccctgacaagtttcttgaagtgtgtacttgggtggatcagattgctgccttgaatgactccaagaccagaaaaaccacctctgagactgtgagggcagttctggatagcctctctgagaagaaaaagtcctctccttag

SEQ ID NO: 50 - DNA, 6233 bp, Artificial Sequence
  Description of Artificial Sequence Synthetic pCLS4421 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcatcgctcagattaaaccaaaccagtctaccaagtttaaacatcgtctacagttgaccctgtacgtgactcaaaagacccagcgccgttggtgtctggacaaactagtggatgaaattggcgttggttacgtagaagattctggtagcgtttcccgttacgttttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccaggtcaatcttacaagttcaaacaccagctctacttgacctttcaagtcactcagaagacacaaagaaggtggttcttggacaaattggttgatcgtattggtgtgggctatgtcagagactctggctctgtgtcaaactacatcctgtctgaagttaagcctcttcataacctgctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttag

SEQ ID NO: 51 - DNA, 6233 bp, Artificial Sequence
  Description of Artificial Sequence Synthetic pCLS4422 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcatcgctcagattaaaccaaaccagtctaccaagtttaaacatcgtctacagttgaccctgtacgtgactcaaaagacccagcgccgttggtgtctggacaaactagtggatgaaattggcgttggttacgtagaagattctggtagcgtttcccgttacgttttaagcaaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccaggtcaatcttacaagttcaaacaccagctctacttgacctttcaagtcactcagaagacacaaagaaggtggttcttggacaaattggttgatcgtattggtgtgggctatgtcagagactctggctctgtgtcaaactacatcctgtctgaagttaagcctcttcataacctgctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttag

SEQ ID NO: 52 - DNA, 6233 bp, Artificial Sequence
  Description of Artificial Sequence Synthetic pCLS4697 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcatcgctcagattaaaccaaaccagtctaccaagtttaaacatcgtctacagttgaccctgtacgtgactcaaaagacccagcgccgttggtgtctggacaaactagtggatgaaattggcgttggttacgtagaagattctggtagcgtttcccgttacgttttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgatgtatcttgctggatttgtggattctgatggctccatcattgctcagataaaaccaaatcaatcttacaagttcaaacaccagctctacttgacctttcaagtcactcagaagacacaaagaaggtggttcttggacaaattggttgatcgtattggtgtgggctatgtcagagactctggctctgtgtcaaactacatcctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcagattgctgccttgaatgactccaagaccagaaaaaacacctctgagactgtgagggcagttctggatagcctctctgagaagaaaaagtcctctccttag

SEQ ID NO: 53 - DNA, 6233 bp, Artificial Sequence
  Description of Artificial Sequence Synthetic pCLS4698 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcatcgctcagattaaaccaaaccagtctaccaagtttaaacatcgtctacagttgaccctgtacgtgactcaaaagacccagcgccgttggtgtctggacaaactagtggatgaaattggcgttggttacgtagaagattctggtagcgtttcccgttacgttttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgatgtatcttgctggatttgtggattctgatggctccatcattgctcagataaaaccaaatcaatcttacaagttcaaacaccagctctacttgacctttcaagtcactcagaagacacaaagaaggtggttcttggacaaattggttgatcgtattggtgtgggctatgtcagagactctggctctgtgtcaaactacatcctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaacacctctgagactgtgagggcagttctggatagcctctctgagaagaaaaagtcctctccttag

SEQ ID NO: 54 - DNA, 6233 bp, Artificial Sequence
  Description of Artificial Sequence Synthetic pCLS4699 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcatcgctcagattaaaccaaaccagtctaccaagtttaaacatcgtctacagttgaccctgtacgtgactcaaaagacccagcgccgttggtgtctggacaaactagtggatgaaattggcgttggttacgtagaagattctggtagcgtttcccgttacgttttaagcaaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgatgtatcttgctggatttgtggattctgatggctccatcattgctcagataaaaccaaatcaatcttacaagttcaaacaccagctctacttgacctttcaagtcactcagaagacacaaagaaggtggttcttggacaaattggttgatcgtattggtgtgggctatgtcagagactctggctctgtgtcaaactacatcctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaacacctctgagactgtgagggcagttctggatagcctctctgagaagaaaaagtcctctccttag

SEQ ID NO: 55 - DNA, 6233 bp, Artificial Sequence
  Description of Artificial Sequence Synthetic pCLS4701 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcatcgctcagattaaaccaaaccagtctaccaagtttaaacatcgtctacagttgaccctgtacgtgactcaaaagacccagcgccgttggtgtctggacaaactagtggatgaaattggcgttggttacgtagaagattctggtagcgtttcccgttacgttttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccaggtcaatcttacaagttcaaacaccagctctacttgacctttcaagtcactcagaagacacaaagaaggtgggttttggacaaattggttgatcgtattggtgtgggctatgtcagagactctggctctgtgtcaaactacatcctgtctgaagttaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttag

SEQ ID NO: 56 - DNA, 6233 bp, Artificial Sequence
  Description of Artificial Sequence Synthetic pCLS4702 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcatcgctcagattaaaccaaaccagtctaccaagtttaaacatcgtctacagttgaccctgtacgtgactcaaaagacccagcgccgttggtgtctggacaaactagtggatgaaattggcgttggttacgtagaagattctggtagcgtttcccgttacgttttaagcaaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccaggtcaatcttacaagttcaaacaccagctctacttgacctttcaagtcactcagaagacacaaagaaggtgggttttggacaaattggttgatcgtattggtgtgggctatgtcagagactctggctctgtgtcaaactacatcctgtctgaagttaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttag

SEQ ID NO: 57 - DNA, 51 bp, Artificial Sequence
  Description of Artificial Sequence Synthetic NANOG2 cloning oligonucleotide oligonucleotide
tggcatacaagtttccaacatcctgaacctcagctacacaatcgtctgtca

SEQ ID NO: 58 - DNA, 51 bp, Artificial Sequence
  Description of Artificial Sequence Synthetic NANOG4 cloning oligonucleotide oligonucleotide
tggcatacaagtttactgaacgctgtaaaatagcttaacaatcgtctgtca

SEQ ID NO: 59 - DNA, 22 bp, Artificial Sequence
  Description of Artificial Sequence Synthetic NANOG2 forward PCR primer
catggatctgcttattcaggac

SEQ ID NO: 60 - DNA, 22 bp, Artificial Sequence
  Description of Artificial Sequence Synthetic NANOG2 reverse PCR primer
agaggcgatgtacggacacata

SEQ ID NO: 61 - DNA, 22 bp, Artificial Sequence
  Description of Artificial Sequence Synthetic NANOG4 forward PCR primer
acctgtgctagtactcatgctt

SEQ ID NO: 62 - DNA, 22 bp, Artificial Sequence
  Description of Artificial Sequence Synthetic NANOG4 reverse PCR primer
cttgatctcagggttgaggctg

SEQ ID NO: 63 - DNA, 30 bp, Artificial Sequence
  Description of Artificial Sequence Synthetic adaptator oligonucleotide
ccatctcatccctgcgtgtctccgactcag

SEQ ID NO: 64 - DNA, 30 bp, Artificial Sequence
  Description of Artificial Sequence Synthetic adaptator oligonucleotide
cctatcccctgtgtgccttggcagtctcag

SEQ ID NO: 65 - protein, 167 aa, Artificial Sequence
  Description of Artificial Sequence Synthetic I-CreI monomer polypeptide
MANTKYNKEFLLYLAGFVDGDGSIIAQIKPNQSYKFKHQLSLTFQVTQKTQRRWFLDKLVDEIGVGYVRDRGSVSBYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 66 - DNA, 24 bp, Artificial Sequence
  Description of Artificial Sequence Synthetic NANOG1 target oligonucleotide
atctgcttattcaggacagccctg

SEQ ID NO: 67 - DNA, 24 bp, Artificial Sequence
  Description of Artificial Sequence Synthetic NANOG3 target oligonucleotide
tataactgtggagaggaatctctg

SEQ ID NO: 68 - DNA, 24 bp, Artificial Sequence
  Description of Artificial Sequence Synthetic NANOG5 target oligonucleotide
attctattatgtgaataattatgt

SEQ ID NO: 69 - DNA, 24 bp, Artificial Sequence
  Description of Artificial Sequence Synthetic NANOG6 target oligonucleotide
atcgcctcttgcaaataatttatg

SEQ ID NO: 70 - DNA, 24 bp, Artificial Sequence
  Description of Artificial Sequence Synthetic NANOG7 target oligonucleotide
attttacaatttctatcatttttt

SEQ ID NO: 71 - DNA, 24 bp, Artificial Sequence
  Description of Artificial Sequence Synthetic NANOG8 target oligonucleotide
ctaatctttgtagaaagaggtctc

SEQ ID NO: 72 - DNA, 64 bp, Artificial Sequence
  Description of Artificial Sequence Synthetic Target 2 oligonucleotide
tgtggatccagcttgtccccaaagcttgccttgctttgaagcatccgactgtaaagaatcttca

SEQ ID NO: 73 - DNA, 62 bp, Artificial Sequence
  Description of Artificial Sequence Synthetic Target 3 oligonucleotide
tccagcttgtccccaaagcttgccttgctttgaagcatccgactgtaaagaatcttcaccta

SEQ ID NO: 74 - DNA, 69 bp, Artificial Sequence
  Description of Artificial Sequence Synthetic Target 4 oligonucleotide
ttgctttgaagcatccgactgtaaagaatcttcacctatgcctgtgatttgtgggcctgaagaaaacta

SEQ ID NO: 75 - DNA, 62 bp, Artificial Sequence
  Description of Artificial Sequence Synthetic Target 6 oligonucleotide
taaagaatcttcacctatgcctgtgatttgtgggcctgaagaaaactatccatccttgcaaa

SEQ ID NO: 76 - DNA, 62 bp, Artificial Sequence
  Description of Artificial Sequence Synthetic Target 7 oligonucleotide
tgggcctgaagaaaactatccatccttgcaaatgtcttctgctgagatgcctcacacggaga

SEQ ID NO: 77 - DNA, 60 bp, Artificial Sequence
  Description of Artificial Sequence Synthetic Target 9 oligonucleotide
tggatctgcttattcaggacagccctgattcttccaccagtcccaaaggcaaacaaccca

SEQ ID NO: 78 - DNA, 65 bp, Artificial Sequence
  Description of Artificial Sequence Synthetic Target 15 oligonucleotide
tggttccagaaccagagaatgaaatctaagaggtggcagaaaaacaactggccgaagaatagcaa

SEQ ID NO: 79 - DNA, 67 bp, Artificial Sequence
  Description of Artificial Sequence Synthetic Target 17 oligonucleotide
tttactcttcctaccaccagggatgcctggtgaacccgactgggaaccttccaatgtggagcaacca

SEQ ID NO: 80 - DNA, 71 bp, Artificial Sequence
  Description of Artificial Sequence Synthetic Target 18 oligonucleotide
tcttcctaccaccagggatgcctggtgaacccgactgggaaccttccaatgtggagcaaccagacctggaa

SEQ ID NO: 81 - DNA, 64 bp, Artificial Sequence
  Description of Artificial Sequence Synthetic Target 20 oligonucleotide
ttccaatgtggagcaaccagacctggaacaattcaacctggagcaaccagacccagaacatcca

SEQ ID NO: 82 - DNA, 64 bp, Artificial Sequence
  Description of Artificial Sequence Synthetic Target 21 oligonucleotide
tccagtcctggagcaaccactcctggaacactcagacctggtgcacccaatcctggaacaatca

SEQ ID NO: 83 - DNA, 62 bp, Artificial Sequence
  Description of Artificial Sequence Synthetic Target 24 oligonucleotide
tgccagtgacttggaggctgccttggaagctgctggggaaggccttaatgtaatacagcaga

SEQ ID NO: 84 - DNA, 7254 bp, Artificial Sequence
  Description of Artificial Sequence Synthetic Plasmid sequence pEF1a-4421 polynucleotide
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacaaacttaagcttggtaccgagctcggatccactagcgatgtacgggccagatatacgcgccaaagctaactgtaggactgagtctattctaaactgaaagcctggacatctggagtaccagggggagatgacgtgttacgggcttccataaaagcagctggctttgaatggaaggagccaagaggccagcacaggagcggattcgtcgctttcacggccatcgagccgaacctctcgcaagtccgtgagccgttaaggaggcccccagtcccgacccttcgccccaagcccctcggggtccccgggcctggtactccttgccacacgggaggggcgcggaagccggggcggaggaggagccaaccccgggctgggctgagacccgcagaggaagacgctctagggatttgtcccggactagcgagatggcaaggctgaggacgggaggctgattgagaggcgaaggtacaccctaatctcaatacaacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgggtttatcttaattaagatatcttatcgataggcgcgcctacacagcggccttgccaccatggccaataccaaatataacgaagagttcctgctgtacctggccggctttgtggacggtgacggtagcatcatcgctcagattaaaccaaaccagtctaccaagtttaaacatcgtctacagttgaccctgtacgtgactcaaaagacccagcgccgttggtgtctggacaaactagtggatgaaattggcgttggttacgtagaagattctggtagcgtttcccgttacgttttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccaggtcaatcttacaagttcaaacaccagctctacttgacctttcaagtcactcagaagacacaaagaaggtggttcttggacaaattggttgatcgtattggtgtgggctatgtcagagactctggctctgtgtcaaactacatcctgtctgaagttaagcctcttcataacctgctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttag

SEQ ID NO: 85 - DNA, 6089 bp, Artificial Sequence
  Description of Artificial Sequence Synthetic pCLS2222 polynucleotide
atggccaataccaaatataacgaagagttcctgctgtacctggccggctttgtggacggtgacggtagcatcatcgctcagattaatccaaaccagtcttctaagtttaaacatcgtctacgtttgaccttttatgtgactcaaaagacccagcgccgttggtttctggacaaactagtggatgaaattggcgttggttacgtacgtgattctggatccgtttcccagtacgttttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaaaccgttcgtgctgtgctggacagcctgagcgggaagaagaaatcctccccggcggccggtggatctgataagtataatcaggctctgtctaaatacaaccaagcactgtccaagtacaatcaggccctgtctggtggaggcggttccaacaaaaagttcctgctgtatcttgctggatttgtggattctgatggctccatcattgctcagataaaaccacgtcaatctaacaagttcaaacaccagctctccttgacttttgcagtcactcagaagacacaaagaaggtggttcttggacaaattggttgataggattggtgtgggctatgtctatgacagtggctctgtgtcagactaccgcctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcagattgctgccttgaatgactccaagaccagaaaaaccacctctgagactgtgagggcagttctggatagcctctctgagaagaaaaagtcctctccttagtctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgacc

SEQ ID NO: 86 - DNA, 22 bp, Artificial Sequence
  Description of Artificial Sequence Synthetic PCR-screen-KI3-F6 primer
ggaggattgggaagacaatagc

SEQ ID NO: 87 - DNA, 24 bp, Artificial Sequence
  Description of Artificial Sequence Synthetic Rag Ex2 R12 primer
ctttcacagtcctgtacatcttgt

SEQ ID NO: 88 - DNA, 643 bp, Artificial Sequence
  Description of Artificial Sequence Synthetic neo probe polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtgg

SEQ ID NO: 89 - DNA, 11224 bp, Artificial Sequence
  Description of Artificial Sequence Synthetic pCLS1399 polynucleotide
ttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggatctaaagctaactgtaggactgagtctattctaaactgaaagcctggacatctggagtaccagggggagatgacgtgttacgggcttccataaaagcagctggctttgaatggaaggagccaagaggccagcacaggagcggattcgtcgctttcacggccatcgagccgaacctctcgcaagtccgtgagccgttaaggaggcccccagtcccgacccttcgccccaagcccctcggggtccccgggcctggtactccttgccacacgggaggggcgcggaagccggggcggaggaggagccaaccccgggctgggctgagacccgcagaggaagacgctctagggatttgtcccggactagcgagatggcaaggctgaggacgggaggctgattgagaggcgaaggtacaccctaatctcaatacaacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggccgatcaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgtacttcaatatagcaatgagcagttaagcgtattactgaaagttccaaagagaaggtttttttaggctaatcgacctcgagcagatccgccaggcgtgtatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggtcctttgtagcataaattactatacttctatagacacgcaaacacaaatacacagcggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtggaattcaagcttcgacggcctctgagctattccagaagtagtgaggaggcttttttggaggcctagagccatggccaaaaacatcaaaaaaaaccaggtaatgaacctcggtccgaactctaaactgctgaaagaatacaaatcccagctgatcgaactgaacatcgaacagttcgaagcaggtatcggtctgatcctgggtgatgcttacatccgttctcgtgatgaaggtaaaacctactgtatgcagttcgagtggaaaaacaaagcatacatggaccacgtatgtctgctgtacgatcagtgggtactgtccccgccgcacaaaaaagaacgtgttaaccacctgggtaacctggtaatcacctggggcgcccagactttcaaacaccaagctttcaacaaactggctaacctgttcatcgttaacaacaaaaaaaccatcccgaacaacctggttgaaaactacctgaccccgatgtctctggcatactggttcatggatgatggtggtaaatgggattacaacaaaaactctaccaacaaatcgatcgtactgaacacccagtctttcactttcgaagaagtagaatacctggttaagggtctgcgtaacaaattccaactgaactgttacgtaaaaatcaacaaaaacaaaccgatcatctacatcgattctatgtcttacctgatcttctacaacctgatcaaaccgtacctgatcccgcagatgatgtacaaactgccgaacactatctcctccgaaactttcctgaaagcggccgcactcgagcaccaccaccaccaccactgagatcgggatccgagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcagttgcatccgacttgtggtctcgctgttccttgggagggtctcctctgagtgattgactaccgcggccctgaagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcagatctattacattatgggtggtatgttggaataaaaatcaactatcatctactaactagtatttacgttactagtatattatcatatacggtgttagaagatgacgcaaatgatgagaaatagtcatctaaattagtggaagctgaaacgcaaggattgataatgtaataggatcaatgaatattaacatataaaatgatgataataatatttatagaattgtgtagaattgcagattcccttttatggattcctaaatcctcgaggagaacttctagtatatctacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagccgcggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccacggaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttaacgacgtagtcgacctcctcttcagtactaaatctaccaataccaaatctgatggaagaatgggctaatgcatcatccttacccagcgcatgtaaaacataagaaggttctagggaagcagatgtacaggctgaacccgaggataatgcgatatcccttagtgccatcaataaagattctccttccacgtaggcgaaagaaacgttaacacaccctggataacgatgatctggagatccgttcaacgtggtatgttcagcggataatagacctttgactaatttatcggatagtcttttgatgtgagcttggtcgttgtcaaattctttcttcatcaatctcgcagcttcaccaaatcccgctaccaatgggggggccaaagtaccagatctgctgcattaatgaatcggccaacgcgcggggagaggcggtttgcgtattgggcgctcttccgcttcctcgctcactgactcgctgcgctcggtcgttcggctgcggcgagcggtatcagcatcgatgaattccacggactatagactatactagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagtgcgaagtagtgatcaggtattgctgttatctgatgagtatacgttgtcctggccacggcagaagcacgcttatcgctccaatttcccacaacattagtcaactccgttaggcccttcattgaaagaaatgaggtcatcaaatgtcttccaatgtgagattttgggccattttttatagcaaagattgaataaggcgcatttttcttcaaagctttattgtacgatctgactaagttatcttttaataattggtattcctgtttattgcttgaagaattgccggtcctatttactcgttttaggactggttcagaattcatcgatgctcactcaaaggtcggtaatacggttatccacagaatcaggggataacgcaggaaagaacatgtgagcaaaaggccagcaaaaggccaggaaccgtaaaaaggccgcgttgctggcgttt

SEQ ID NO: 90 - DNA, 3890 bp, Artificial Sequence
  Description of Artificial Sequence Synthetic Matrix 1 polynucleotide
ccgcggcgcgccgccatgttggccaggctggtttcaaactcctgacttcaggtgatccgcctgccacggcctcccaatttactgggattacaggggtgggccaccgcgcccggcctttttcttaatttttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacgctagggataacagggtaatatatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtaaaattccgcccctctcccccccccccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataaatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagccgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagggataacagggtaatataaagcgcatttacattatgactacagttgtaaagagtaggttgtggaaaaggaataagtaaacatgattgctatatttgagcctcagatttgaatgtcaccctgcttttcctcttttttagaatcagggactcactttgtcacataggctggagtgcaatggtgtgatcatggatcaccgcagcctcaaccctgagatcaagtgatcctcttgcctcagcctcccaactactaggacaagaggcgtgtgtcactatgcccagctaattaaaaaattatttttgtagagacagggtctcactttgcagctcgggctggtcttgaactcctgacctcaagtgatcctccaccctcagcttcccaagtcgctgggattacaggcgctgacccactctgcatggctattcacccagtttttctaaactatgcattaatgttcttgcctttataatttctttctttctttctttctttttttttttttgagacagggtctcattctgttgcccacgcgggagtgcagtggcgtgatcttggctcactgcaacctccgcctcctaggttcaggtgatttctcctgtctcagtctctcgagtagcaggtattacagatgtatgccaccacgcctagctaatttttgtagttcttagtagggccggcctgcaggttcg

SEQ ID NO: 91 - DNA, 3466 bp, Artificial Sequence
  Description of Artificial Sequence Synthetic Matrix 2 polynucleotide
ccgcggcgcgccgccatgttggccaggctggtttcaaactcctgacttcaggtgatccgcctgccacggcctcccaatttactgggattacaggggtgggccaccgcgcccggcctttttcttaatttttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacgctagggataacagggtaatatatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtaaaattccgcccctctcccccccccccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataaatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagggataacagggtaatatgtaaagcgcatttacattatgactacagttgtaaagagtaggttgtggaaaaggaataagtaaacatgattgctatatttgagcctcagatttgaatgtcaccctgcttttcctcttttttagaatcagggactcactttgtcacataggctggagtgcaatggtgtgatcatggatcaccgcagcctcaaccctgagatcaagtgatcctcttgcctcagcctcccaactactaggacaagaggcgtgtgtcactatgcccagctaattaaaaaattatttttgtagagacagggtctcactttgcagctcgggctggtcttgaactcctgacctcaagtgatcctccaccctcagcttcccaagtcgctgggattacaggcgctgacccactctgcatggctattcacccagtttttctaaactatgcattaatgttcttgcctttataatttctttctttctttctttctttttttttttttgagacagggtctcattctgttgcccacgcgggagtgcagtggcgtgatcttggctcactgcaacctccgcctcctaggttcaggtgatttctcctgtctcagtctctcgagtagcaggtattacagatgtatgccaccacgcctagctaatttttgtagttcttagtagggccggcctgcaggttcg

SEQ ID NO: 92 - DNA, 3344 bp, Artificial Sequence
  Description of Artificial Sequence Synthetic Matrix 3 polynucleotide
ccgcggcgcgccgccatgttggccaggctggtttcaaactcctgacttcaggtgatccgcctgccacggcctcccaatttactgggattacaggggtgggccaccgcgcccggcctttttcttaatttttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacgctagggataacagggtaatatatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacagggagggcagaggaagtcttctaacatgcggtgacgtggaggagaatcccggccctaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagccgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagggataacagggtaatatgtaaagcgcatttacattatgactacagttgtaaagagtaggttgtggaaaaggaataagtaaacatgattgctatatttgagcctcagatttgaatgtcaccctgcttttcctcttttttagaatcagggactcactttgtcacataggctggagtgcaatggtgtgatcatggatcaccgcagcctcaaccctgagatcaagtgatcctcttgcctcagcctcccaactactaggacaagaggcgtgtgtcactatgcccagctaattaaaaaattatttttgtagagacagggtctcactttgcagctcgggctggtcttgaactcctgacctcaagtgatcctccaccctcagcttcccaagtcgctgggattacaggcgctgacccactctgcatggctattcacccagtttttctaaactatgcattaatgttcttgcctttataatttctttctttctttctttctttttttttttttgagacagggtctcattctgttgcccacgcgggagtgcagtggcgtgatcttggctcactgcaacctccgcctcctaggttcaggtgatttctcctgtctcagtctctcgagtagcaggtattacagatgtatgccaccacgcctagctaatttttgtagttcttagtagggccggcctgcaggttcg

SEQ ID NO: 93 - DNA, 2918 bp, Artificial Sequence
  Description of Artificial Sequence Synthetic Matrix 4 polynucleotide
ccgcggcgcgccgccatgttggccaggctggtttcaaactcctgacttcaggtgatccgcctgccacggcctcccaatttactgggattacaggggtgggccaccgcgcccggcctttttcttaatttttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacgctagggataacagggtaatatatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacagggagggcagaggaagtcttctaacatgcggtgacgtggaggagaatcccggccctaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagggataacagggtaatatgtaaagcgcatttacattatgactacagttgtaaagagtaggttgtggaaaaggaataagtaaacatgattgctatatttgagcctcagatttgaatgtcaccctgcttttcctcttttttagaatcagggactcactttgtcacataggctggagtgcaatggtgtgatcatggatcaccgcagcctcaaccctgagatcaagtgatcctcttgcctcagcctcccaactactaggacaagaggcgtgtgtcactatgcccagctaattaaaaaattatttttgtagagacagggtctcactttgcagctcgggctggtcttgaactcctgacctcaagtgatcctccaccctcagcttcccaagtcgctgggattacaggcgctgacccactctgcatggctattcacccagtttttctaaactatgcattaatgttcttgcctttataatttctttctttctttctttctttttttttttttgagacagggtctcattctgttgcccacgcgggagtgcagtggcgtgatcttggctcactgcaacctccgcctcctaggttcaggtgatttctcctgtctcagtctctcgagtagcaggtattacagatgtatgccaccacgcctagctaatttttgtagttcttagtagggccggcctgcaggttcg

SEQ ID NO: 94 - DNA, 4154 bp, Artificial Sequence
  Description of Artificial Sequence Synthetic Matrix 5 polynucleotide
ccgcggcgcgccgccatgttggccaggctggtttcaaactcctgacttcaggtgatccgcctgccacggcctcccaatttactgggattacaggggtgggccaccgcgcccggcctttttcttaatttttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacgctagggataacagggtaatatatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtaaaattccgcccctctcccccccccccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataaatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagccgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagggataacagggtaatattccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactggtaaagcgcatttacattatgactacagttgtaaagagtaggttgtggaaaaggaataagtaaacatgattgctatatttgagcctcagatttgaatgtcaccctgcttttcctcttttttagaatcagggactcactttgtcacataggctggagtgcaatggtgtgatcatggatcaccgcagcctcaaccctgagatcaagtgatcctcttgcctcagcctcccaactactaggacaagaggcgtgtgtcactatgcccagctaattaaaaaattatttttgtagagacagggtctcactttgcagctcgggctggtcttgaactcctgacctcaagtgatcctccaccctcagcttcccaagtcgctgggattacaggcgctgacccactctgcatggctattcacccagtttttctaaactatgcattaatgttcttgcctttataatttctttctttctttctttctttttttttttttgagacagggtctcattctgttgcccacgcgggagtgcagtggcgtgatcttggctcactgcaacctccgcctcctaggttcaggtgatttctcctgtctcagtctctcgagtagcaggtattacagatgtatgccaccacgcctagctaatttttgtagttcttagtagggccggcctgcaggttcg

SEQ ID NO: 95 - DNA, 3728 bp, Artificial Sequence
  Description of Artificial Sequence Synthetic Matrix 6 polynucleotide
ccgcggcgcgccgccatgttggccaggctggtttcaaactcctgacttcaggtgatccgcctgccacggcctcccaatttactgggattacaggggtgggccaccgcgcccggcctttttcttaatttttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacgctagggataacagggtaatatatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtaaaattccgcccctctcccccccccccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataaatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagggataacagggtaatattccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactggtaaagcgcatttacattatgactacagttgtaaagagtaggttgtggaaaaggaataagtaaacatgattgctatatttgagcctcagatttgaatgtcaccctgcttttcctcttttttagaatcagggactcactttgtcacataggctggagtgcaatggtgtgatcatggatcaccgcagcctcaaccctgagatcaagtgatcctcttgcctcagcctcccaactactaggacaagaggcgtgtgtcactatgcccagctaattaaaaaattatttttgtagagacagggtctcactttgcagctcgggctggtcttgaactcctgacctcaagtgatcctccaccctcagcttcccaagtcgctgggattacaggcgctgacccactctgcatggctattcacccagtttttctaaactatgcattaatgttcttgcctttataatttctttctttctttctttctttttttttttttgagacagggtctcattctgttgcccacgcgggagtgcagtggcgtgatcttggctcactgcaacctccgcctcctaggttcaggtgatttctcctgtctcagtctctcgagtagcaggtattacagatgtatgccaccacgcctagctaatttttgtagttcttagtagggccggcctgcaggttcg

SEQ ID NO: 96 - DNA, 3606 bp, Artificial Sequence
  Description of Artificial Sequence Synthetic Matrix 7 polynucleotide
ccgcggcgcgccgccatgttggccaggctggtttcaaactcctgacttcaggtgatccgcctgccacggcctcccaatttactgggattacaggggtgggccaccgcgcccggcctttttcttaatttttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacgctagggataacagggtaatatatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacagggagggcagaggaagtcttctaacatgcggtgacgtggaggagaatcccggccctaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagccgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagggataacagggtaatattccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactggtaaagcgcatttacattatgactacagttgtaaagagtaggttgtggaaaaggaataagtaaacatgattgctatatttgagcctcagatttgaatgtcaccctgcttttcctcttttttagaatcagggactcactttgtcacataggctggagtgcaatggtgtgatcatggatcaccgcagcctcaaccctgagatcaagtgatcctcttgcctcagcctcccaactactaggacaagaggcgtgtgtcactatgcccagctaattaaaaaattatttttgtagagacagggtctcactttgcagctcgggctggtcttgaactcctgacctcaagtgatcctccaccctcagcttcccaagtcgctgggattacaggcgctgacccactctgcatggctattcacccagtttttctaaactatgcattaatgttcttgcctttataatttctttctttctttctttctttttttttttttgagacagggtctcattctgttgcccacgcgggagtgcagtggcgtgatcttggctcactgcaacctccgcctcctaggttcaggtgatttctcctgtctcagtctctcgagtagcaggtattacagatgtatgccaccacgcctagctaatttttgtagttcttagtagggccggcctgcaggttcg

SEQ ID NO: 97 - DNA, 3180 bp, Artificial Sequence
  Description of Artificial Sequence Synthetic Matrix 8 polynucleotide
ccgcggcgcgccgccatgttggccaggctggtttcaaactcctgacttcaggtgatccgcctgccacggcctcccaatttactgggattacaggggtgggccaccgcgcccggcctttttcttaatttttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacgctagggataacagggtaatatatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacagggagggcagaggaagtcttctaacatgcggtgacgtggaggagaatcccggccctaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagggataacagggtaatattccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactggtaaagcgcatttacattatgactacagttgtaaagagtaggttgtggaaaaggaataagtaaacatgattgctatatttgagcctcagatttgaatgtcaccctgcttttcctcttttttagaatcagggactcactttgtcacataggctggagtgcaatggtgtgatcatggatcaccgcagcctcaaccctgagatcaagtgatcctcttgcctcagcctcccaactactaggacaagaggcgtgtgtcactatgcccagctaattaaaaaattatttttgtagagacagggtctcactttgcagctcgggctggtcttgaactcctgacctcaagtgatcctccaccctcagcttcccaagtcgctgggattacaggcgctgacccactctgcatggctattcacccagtttttctaaactatgcattaatgttcttgcctttataatttctttctttctttctttctttttttttttttgagacagggtctcattctgttgcccacgcgggagtgcagtggcgtgatcttggctcactgcaacctccgcctcctaggttcaggtgatttctcctgtctcagtctctcgagtagcaggtattacagatgtatgccaccacgcctagctaatttttgtagttcttagtagggccggcctgcaggttcg

SEQ ID NO: 98 - DNA, 5030 bp, Artificial Sequence
  Description of Artificial Sequence Synthetic Matrix 9 polynucleotide
ccgcggcgcgccgccatgttggccaggctggtttcaaactcctgacttcaggtgatccgcctgccacggcctcccaatttactgggattacaggggtgggccaccgcgcccggcctttttcttaatttttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacgctagggataacagggtaatatatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtaaaattccgcccctctcccccccccccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataaatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagccgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagggataacagggtaatattccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactggtaagaaagaaatttatccttgaaaggccaagttccttaagggaaaagagagaaggagagagggttaagggatcatttccctcttgagcaatgatggaccattactataaagaagtgttattatcaactaatcctctggaaacccctttttccattataacttggtggcacctgccctttgaactatgtcccaggtctcaggagtgtgcattgagttgaaggacacagaattcggcagttgaacagtgtgcagtaagtttgagaacctatgggcttaggcatggtggaaacaaaaatgtatcgttatagttaaatgaaggtgatgtgtacatcttcacatagtgctggacacatgtgaataaatagcagatttattgctaattagccagaagacctaacgtcatagctcagggatgagcatgattttgttttgccaaaaatggcatggcaaatcacgatgagatttctgtaatacataatttgggtaattctttctatgtcagtaacggctgtctcttctccattctctgggtttgtggatgttactgggcagctctgagtttgggagcacctcccatgtctaattctcctaagtcctgggaagcgttgacccaactttatggtaaagataattccagaaagtttaatctactgacagtcaaacagaatgtagctagaagtccagtttggcttcaaaacctgtgctagtactcatgcttctgactggtagctgcaaggggtgggggatactcgggatactcataaagccgctaccacttttttgaaaatcaatttttcagtagttttcaaaaacttgagaatgaaccaactttaccaagaatgccattggtaacactgaacgctcccaaatagcttaaaaagcgcatttacattatgactacagttgtaaagagtaggttgtggaaaaggaataagtaaacatgattgctatatttgagcctcagatttgaatgtcaccctgcttttcctcttttttagaatcagggactcactttgtcacataggctggagtgcaatggtgtgatcatggatcaccgcagcctcaaccctgagatcaagtgatcctcttgcctcagcctcccaactactaggacaagaggcgtgtgtcactatgcccagctaattaaaaaattatttttgtagagacagggtctcactttgcagctcgggctggtcttgaactcctgacctcaagtgatcctccaccctcagcttcccaagtcgctgggattacaggcgctgacccactctgcatggctattcacccagtttttctaaactatgcattaatgttcttgcctttataatttctttctttctttctttctttttttttttttgagacagggtctcattctgttgcccacgcgggagtgcagtggcgtgatcttggctcactgcaacctccgcctcctaggttcaggtgatttctcctgtctcagtctctcgagtagcaggtattacagatgtatgccaccacgcctagctaatttttgtagttcttagtagggccggcctgcaggttcg

SEQ ID NO: 99 - DNA, 4604 bp, Artificial Sequence
  Description of Artificial Sequence Synthetic Matrix 10 polynucleotide
ccgcggcgcgccgccatgttggccaggctggtttcaaactcctgacttcaggtgatccgcctgccacggcctcccaatttactgggattacaggggtgggccaccgcgcccggcctttttcttaatttttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacgctagggataacagggtaatatatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtaaaattccgcccctctcccccccccccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataaatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagggataacagggtaatattccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactggtaagaaagaaatttatccttgaaaggccaagttccttaagggaaaagagagaaggagagagggttaagggatcatttccctcttgagcaatgatggaccattactataaagaagtgttattatcaactaatcctctggaaacccctttttccattataacttggtggcacctgccctttgaactatgtcccaggtctcaggagtgtgcattgagttgaaggacacagaattcggcagttgaacagtgtgcagtaagtttgagaacctatgggcttaggcatggtggaaacaaaaatgtatcgttatagttaaatgaaggtgatgtgtacatcttcacatagtgctggacacatgtgaataaatagcagatttattgctaattagccagaagacctaacgtcatagctcagggatgagcatgattttgttttgccaaaaatggcatggcaaatcacgatgagatttctgtaatacataatttgggtaattctttctatgtcagtaacggctgtctcttctccattctctgggtttgtggatgttactgggcagctctgagtttgggagcacctcccatgtctaattctcctaagtcctgggaagcgttgacccaactttatggtaaagataattccagaaagtttaatctactgacagtcaaacagaatgtagctagaagtccagtttggcttcaaaacctgtgctagtactcatgcttctgactggtagctgcaaggggtgggggatactcgggatactcataaagccgctaccacttttttgaaaatcaatttttcagtagttttcaaaaacttgagaatgaaccaactttaccaagaatgccattggtaacactgaacgctcccaaatagcttaaaaagcgcatttacattatgactacagttgtaaagagtaggttgtggaaaaggaataagtaaacatgattgctatatttgagcctcagatttgaatgtcaccctgcttttcctcttttttagaatcagggactcactttgtcacataggctggagtgcaatggtgtgatcatggatcaccgcagcctcaaccctgagatcaagtgatcctcttgcctcagcctcccaactactaggacaagaggcgtgtgtcactatgcccagctaattaaaaaattatttttgtagagacagggtctcactttgcagctcgggctggtcttgaactcctgacctcaagtgatcctccaccctcagcttcccaagtcgctgggattacaggcgctgacccactctgcatggctattcacccagtttttctaaactatgcattaatgttcttgcctttataatttctttctttctttctttctttttttttttttgagacagggtctcattctgttgcccacgcgggagtgcagtggcgtgatcttggctcactgcaacctccgcctcctaggttcaggtgatttctcctgtctcagtctctcgagtagcaggtattacagatgtatgccaccacgcctagctaatttttgtagttcttagtagggccggcctgcaggttcg

SEQ ID NO: 100 - DNA, 4482 bp, Artificial Sequence
  Description of Artificial Sequence Synthetic Matrix 11 polynucleotide
ccgcggcgcgccgccatgttggccaggctggtttcaaactcctgacttcaggtgatccgcctgccacggcctcccaatttactgggattacaggggtgggccaccgcgcccggcctttttcttaatttttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacgctagggataacagggtaatatatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacagggagggcagaggaagtcttctaacatgcggtgacgtggaggagaatcccggccctaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagccgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagggataacagggtaatattccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactggtaagaaagaaatttatccttgaaaggccaagttccttaagggaaaagagagaaggagagagggttaagggatcatttccctcttgagcaatgatggaccattactataaagaagtgttattatcaactaatcctctggaaacccctttttccattataacttggtggcacctgccctttgaactatgtcccaggtctcaggagtgtgcattgagttgaaggacacagaattcggcagttgaacagtgtgcagtaagtttgagaacctatgggcttaggcatggtggaaacaaaaatgtatcgttatagttaaatgaaggtgatgtgtacatcttcacatagtgctggacacatgtgaataaatagcagatttattgctaattagccagaagacctaacgtcatagctcagggatgagcatgattttgttttgccaaaaatggcatggcaaatcacgatgagatttctgtaatacataatttgggtaattctttctatgtcagtaacggctgtctcttctccattctctgggtttgtggatgttactgggcagctctgagtttgggagcacctcccatgtctaattctcctaagtcctgggaagcgttgacccaactttatggtaaagataattccagaaagtttaatctactgacagtcaaacagaatgtagctagaagtccagtttggcttcaaaacctgtgctagtactcatgcttctgactggtagctgcaaggggtgggggatactcgggatactcataaagccgctaccacttttttgaaaatcaatttttcagtagttttcaaaaacttgagaatgaaccaactttaccaagaatgccattggtaacactgaacgctcccaaatagcttaaaaagcgcatttacattatgactacagttgtaaagagtaggttgtggaaaaggaataagtaaacatgattgctatatttgagcctcagatttgaatgtcaccctgcttttcctcttttttagaatcagggactcactttgtcacataggctggagtgcaatggtgtgatcatggatcaccgcagcctcaaccctgagatcaagtgatcctcttgcctcagcctcccaactactaggacaagaggcgtgtgtcactatgcccagctaattaaaaaattatttttgtagagacagggtctcactttgcagctcgggctggtcttgaactcctgacctcaagtgatcctccaccctcagcttcccaagtcgctgggattacaggcgctgacccactctgcatggctattcacccagtttttctaaactatgcattaatgttcttgcctttataatttctttctttctttctttctttttttttttttgagacagggtctcattctgttgcccacgcgggagtgcagtggcgtgatcttggctcactgcaacctccgcctcctaggttcaggtgatttctcctgtctcagtctctcgagtagcaggtattacagatgtatgccaccacgcctagctaatttttgtagttcttagtagggccggcctgcaggttcg

SEQ ID NO: 101 - DNA, 4056 bp, Artificial Sequence
  Description of Artificial Sequence Synthetic Matrix 12 polynucleotide
ccgcggcgcgccgccatgttggccaggctggtttcaaactcctgacttcaggtgatccgcctgccacggcctcccaatttactgggattacaggggtgggccaccgcgcccggcctttttcttaatttttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacgctagggataacagggtaatatatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacagggagggcagaggaagtcttctaacatgcggtgacgtggaggagaatcccggccctaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagggataacagggtaatattccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactggtaagaaagaaatttatccttgaaaggccaagttccttaagggaaaagagagaaggagagagggttaagggatcatttccctcttgagcaatgatggaccattactataaagaagtgttattatcaactaatcctctggaaacccctttttccattataacttggtggcacctgccctttgaactatgtcccaggtctcaggagtgtgcattgagttgaaggacacagaattcggcagttgaacagtgtgcagtaagtttgagaacctatgggcttaggcatggtggaaacaaaaatgtatcgttatagttaaatgaaggtgatgtgtacatcttcacatagtgctggacacatgtgaataaatagcagatttattgctaattagccagaagacctaacgtcatagctcagggatgagcatgattttgttttgccaaaaatggcatggcaaatcacgatgagatttctgtaatacataatttgggtaattctttctatgtcagtaacggctgtctcttctccattctctgggtttgtggatgttactgggcagctctgagtttgggagcacctcccatgtctaattctcctaagtcctgggaagcgttgacccaactttatggtaaagataattccagaaagtttaatctactgacagtcaaacagaatgtagctagaagtccagtttggcttcaaaacctgtgctagtactcatgcttctgactggtagctgcaaggggtgggggatactcgggatactcataaagccgctaccacttttttgaaaatcaatttttcagtagttttcaaaaacttgagaatgaaccaactttaccaagaatgccattggtaacactgaacgctcccaaatagcttaaaaagcgcatttacattatgactacagttgtaaagagtaggttgtggaaaaggaataagtaaacatgattgctatatttgagcctcagatttgaatgtcaccctgcttttcctcttttttagaatcagggactcactttgtcacataggctggagtgcaatggtgtgatcatggatcaccgcagcctcaaccctgagatcaagtgatcctcttgcctcagcctcccaactactaggacaagaggcgtgtgtcactatgcccagctaattaaaaaattatttttgtagagacagggtctcactttgcagctcgggctggtcttgaactcctgacctcaagtgatcctccaccctcagcttcccaagtcgctgggattacaggcgctgacccactctgcatggctattcacccagtttttctaaactatgcattaatgttcttgcctttataatttctttctttctttctttctttttttttttttgagacagggtctcattctgttgcccacgcgggagtgcagtggcgtgatcttggctcactgcaacctccgcctcctaggttcaggtgatttctcctgtctcagtctctcgagtagcaggtattacagatgtatgccaccacgcctagctaatttttgtagttcttagtagggccggcctgcaggttcg

SEQ ID NO: 102 - protein, 9 aa, Unknown
  Description of Unknown “LAGLIDADG” family motif peptide
LAGLIDADG